(19) United States
(12) Patent Application Publication
(10) Pub. No.: US 2008/0039473 A1
(43) Pub. Date: Feb. 14, 2008

(54) PREPARATION AND UTILITY OF SUBSTITUTED QUINAZOLINE COMPOUNDS WITH ALPHA-ADRENERGIC BLOCKING EFFECTS

(75) Inventors: Thomas G. GANT, Carlsbad, CA (US); Sepehr SARSHAR, Cardiff by the Sea, CA (US)

Correspondence Address: WILSON SONSINI GOODRICH & ROSATI, 650 PAGE MILL ROAD, PALO ALTO, CA, 94304-1050, US

(73) Assignee: AUSPEX PHARMACEUTICALS, INC., Vista, CA (US)

(21) Appl. No.: 11/835,850

(22) Filed: Aug. 8, 2007

Related U.S. Application Data
(60) Provisional application No. 60/836,643, filed on Aug. 8, 2006.

(51) Int. Cl. A61K 31/517; A61P 29/00; A61P 35/00; A61P 9/00; C07D 405/12; C07D 405/14

(52) U.S. Cl. 514/252.17; 514/266.24; 544/284; 544/291

(57) ABSTRACT

The present disclosure is directed to modulators of alpha-adrenergic receptors and pharmaceutically acceptable salts and prodrugs thereof, the chemical synthesis thereof, and the use of such compounds for the treatment and/or management of hypertension, cardiac failure, prostatitis, and benign prostatic hyperplasia, and any other condition in which it is beneficial to modulate an alpha-adrenergic receptor.

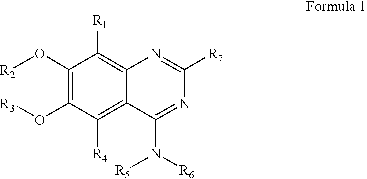

### RELATED APPLICATIONS

This application claims the benefit of U.S. Provisional Patent Application No. 60/836,643 filed Aug. 8, 2006, which is herein incorporated by reference in its entirety.

### FIELD

The present disclosure is directed to alpha-adrenergic receptor modulators and pharmaceutically acceptable salts and prodrugs thereof, the chemical synthesis thereof, and the use of such compounds for the treatment and/or management of hypertension, cardiac failure, prostatitis, and/or benign prostatic hyperplasia.

### BACKGROUND

Doxazosin (Cardura®) is a therapeutic agent believed to improve cardiovascular endpoints through interaction with adrenergic receptors, most particularly through antagonism of the α-subtype, hence the common name: α-blockers. As such, Doxazosin belongs to a large class of such agents including terazosin (Hytrin®), alfusosin (Uroxatral®), tamsulosin (Flomax®), and the first approved member of the alpha-selective class prazosin (Minipress®). The various agents differ in pharmacology in part based on selectivity profiles for the adrenergic α-subtypes. This in turn is believed to account for the observed uroselectivity that has given some agents priority in treating benign prostatic hyperplasia (BPH). Treatment of hypertension has increasingly turned toward polypharmacy since the disease has heterogeneous origins. This is reflected in the wide interpatient variability in response to monotherapies. Co-administering antihypertensive therapies is thought to have several benefits, including enhanced efficacy, tolerability, compliance and, in some cases, potentially beneficial changes in biochemical variables such as improvements in lipid profiles. Synergistic effects have been seen when an α-blocker is administered together with either a calcium channel blocker or angiotensin-converting enzyme inhibitor. It is thought that α-blockers counteract side effects observed with β-adrenergic blockers—including a moderating effect on high density lipoprotein and serum triglyceride levels.

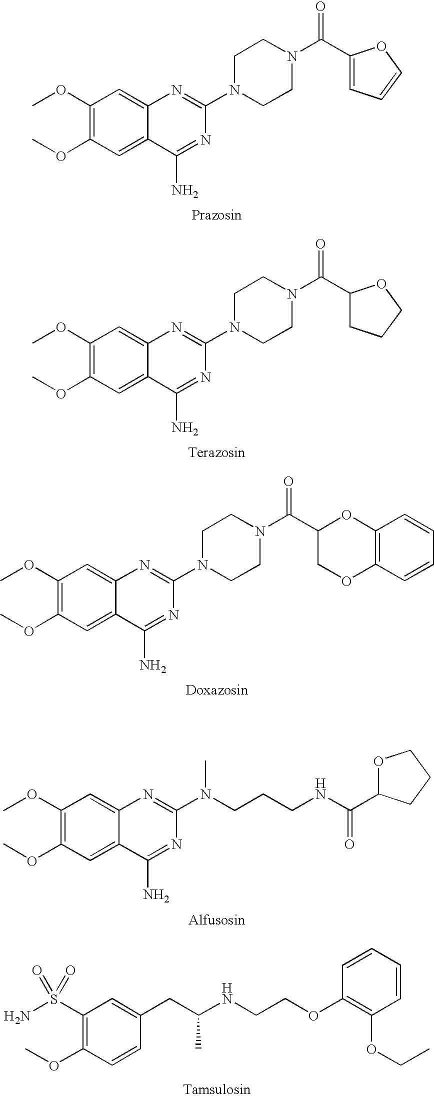

### SUMMARY OF THE INVENTION

Described herein are deuterated alpha-adrenergic receptor modulators. In one embodiment, the deuterium enrichment occurs at a specific position on the modulator. In one embodiment, the deuterium enrichment is no less than about 1%. In a firther embodiment, the deuterium enrichment is no less than about 10%. In a further embodiment, the deuterium enrichment is no less than about 20%. In a further embodiment, the deuterium enrichment is no less than about 50%. In a further embodiment, the deuterium enrichment is no less than about 70%. In a further embodiment, the deuterium enrichment is no less than about 80%. In a further embodiment, the deuterium enrichment is no less than about 90%. In a further embodiment, the deuterium enrichment is no less than about 95%. In one embodiment, the deuterated modulator has a slower rate of metabolism than the corresponding protiated modulator.

Further described herein are deuterated substituted quinazoline compounds, including, for each of the aforementioned compounds, a single enantiomer, a mixture of a (+)-enantiomer and a (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug. In one embodiment, the deuterium enrichment occurs at a specific position on the compound. In one embodiment, the deuterium enrichment is no less than about 1%. In a further embodiment, the deuterium enrichment is no less than about 10%. In a further embodiment, the deuterium enrichment is no less than about 20%. In a further embodiment, the deuterium enrichment is no less than about 50%. In a further embodiment, the deuterium enrichment is no less than about 70%. In a further embodiment, the deuterium enrichment is no less than about 80%. In a further embodiment, the deuterium enrichment is no less than about 90%. In a further embodiment, the deuterium enrichment is no less than about 95%. In one embodiment, the deuterated compound has a slower rate of metabolism than the corresponding protiated compound.

Provided herein are compounds of Formula 1:

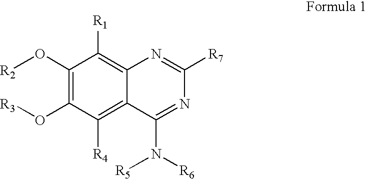

or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers, or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of:

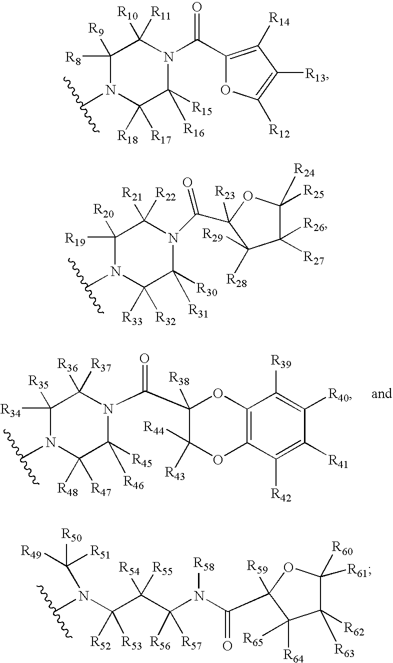

wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium; provided that compounds of Formula 1 contain at least one deuterium atom, and that deuterium enrichment in compounds of Formula 1 is at least about 1%; with the proviso that compounds of Formula 1 cannot be selected from the group consisting of:

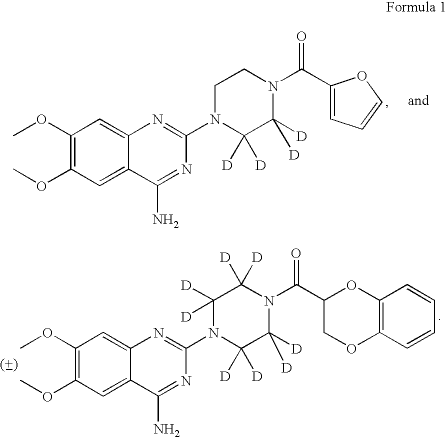

Also provided herein are pharmaceutical compositions comprising a compound of Formula 1 described herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; and one or more pharmaceutically acceptable excipients or carriers.

Further, provided is a method of modulating an alpha-adrenergic receptor which comprises administering to a subject a therapeutically effective amount of a compound described herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof.

Additionally, provided herein are methods of treating, preventing, or ameliorating one or more symptoms of a disease or condition selected from the group consisting of hypertension, cardiac failure, prostatitis, and benign prostatic hyperplasia, and/or any other condition in which it is beneficial to modulate an alpha-adrenergic receptor which comprises administering to a subject a therapeutically effective amount of a compound described herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof.

Provided herein is a method of treating a mammal suffering from a disease or condition in which it is beneficial to modulate an alpha-adrenergic receptor, comprising administering to said mammal a therapeutically effective amount of a compound of Formula 1 so as to affect decreased inter-individual variation in plasma levels of said compound or a metabolite thereof as compared to the non-isotopically enriched compound.

Also provided herein is a method of treating a mammal suffering from a disease or condition in which it is beneficial to modulate an alpha-adrenergic receptor, comprising administering to said mammal a therapeutically effective amount of a compound of Formula 1 so as to affect increased average plasma levels of said compound per dosage unit thereof as compared to the non-isotopically enriched compound.

In another aspect is a method of treating a mammal suffering from a disease or condition in which it is beneficial to modulate an alpha-adrenergic receptor, comprising administering a therapeutically effective amount of a compound of Formula 1 so as to affect decreased average plasma levels of at least one metabolite of said compound per dosage unit thereof as compared to the non-isotopically enriched compound.

In one aspect is a method of treating a mammal suffering from a disease or condition in which it is beneficial to modulate an alpha-adrenergic receptor, comprising administering a therapeutically effective amount of a compound of Formula 1 so as to affect a decreased metabolism by at least one polymorphically-expressed cytochrome P₄₅₀ isoform in mammalian subjects per dosage unit thereof as compared to the non-isotopically enriched compound.

In one embodiment is a method of treating a mammal suffering from a disease or condition in which it is beneficial to modulate an alpha-adrenergic receptor, comprising administering a therapeutically effective amount of a compound of Formula 1, so as to affect a decreased metabolism by at least one polymorphically-expressed cytochrome P₄₅₀ isoform in mammalian subjects per dosage unit thereof as compared to the non-isotopically enriched compound, wherein at least one polymorphically-expressed cytochrome P₄₅₀ isoform is selected from the group consisting of CYP2C8, CYP2C9, CYP2C19, and CYP2D6.

In one aspect is a method of treating a mammal suffering from a disease or condition in which it is beneficial to modulate an alpha-adrenergic receptor, comprising administering a therapeutically effective amount of a compound of Formula 1 so as to affect a decreased inhibition of at least one cytochrome P₄₅₀ isoform in mammalian subjects per dosage unit thereof as compared to the non-isotopically enriched compound.

In another aspect is a method of treating a mammal suffering from a disease or condition in which it is beneficial to modulate an alpha-adrenergic receptor, comprising administering a therapeutically effective amount of a compound of Formula 1, so as to affect a decreased inhibition of at least one cytochrome P₄₅₀ isoform in mammalian subjects per dosage unit thereof as compared to the non-isotopically enriched compound, wherein at least one cytochrome P₄₅₀ isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In a further aspect is a method of treating a mammal suffering from a disease or condition in which it is beneficial to modulate an alpha-adrenergic receptor, comprising administering a therapeutically effective amount of a compound of Formula 1 so as to elicit an improved clinical effect during the treatment in said mammal per dosage unit thereof as compared to the non-isotopically enriched compound.

Also provided herein is a method of treating a mammal suffering from a disease or condition in which it is beneficial to modulate an alpha-adrenergic receptor, comprising administering to said mammal a therapeutically effective amount of a compound of Formula 1.

Also provided herein are articles of manufacture and kits containing compounds described herein. By way of example only a kit or article of manufacture can include a container (such as a bottle) with a desired amount of a compound (or pharmaceutical composition of a compound) described herein. Such a kit or article of manufacture can further include instructions for using the compound (or pharmaceutical composition of a compound) described herein. The instructions can be attached to the container, or can be included in a package (such as a box or a plastic or foil bag) holding the container.

In another aspect is the use of a compound described herein in the manufacture of a medicament for treating a disease or condition in an animal in which an alpha-adrenergic receptor contributes to the pathology and/or symptomology of the disease or condition.

In another aspect are processes for preparing a compound described herein as alpha-adrenergic receptor modulators, or other pharmaceutically acceptable derivatives such as prodrug derivatives, or individual isomers and mixture of isomers or enantiomers thereof.

### INCORPORATION BY REFERENCE

All publications, patents, and patent applications mentioned in this specification are herein incorporated by reference to the same extent as if each individual publication or patent application was specifically and individually indicated to be incorporated by reference.

### DETAILED DESCRIPTION

To facilitate understanding of the disclosure set forth herein, a number of terms are defined below.

As used herein, the singular forms “a,” “an,” and “the” may refer to plural articles unless specifically stated otherwise. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term herein, those in this section prevail unless stated otherwise.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The terms “prevent,” “preventing,” and “prevention” refer to a method of delaying or precluding the onset of a disorder, disease, or condition; and/or its attendant symptoms, barring a subject from acquiring a disease or reducing a subject's risk of acquiring a disorder, disease, or condition.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “deuterium enrichment” refers to the percentage of incorporation of deuterium at a given position in a molecule in the place of hydrogen. For example, deuterium enrichment of about 1% at a given position means that about 1% of molecules in a given sample contain deuterium at the specified position. Because the naturally occurring distribution of deuterium is about 0.0156%, deuterium enrichment at any positions in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% of the molecules are a single compound, including a racemic mixture or single stereoisomer thereof, as determined by standard analytical methods.

The term “about” or “approximately” means an acceptable error for a particular value as determined by one of ordinary skill in the art, which depends in part on how the value is measured or determined. In certain embodiments, “about” can mean with 1 or more standard deviations.

The terms “active ingredient” and “active substance” refer to a compound, which is administered, alone or with one or more pharmaceutically acceptable excipients, to a subject for treating, preventing, or ameliorating one or more symptoms of a disorder or disease.

The terms “drug,” “therapeutic agent,” and “chemotherapeutic agent” refer to a compound, or a pharmaceutical composition thereof, which is administered to a subject for treating, preventing, or ameliorating one or more symptoms of a disorder or disease.

The term “release controlling excipient” refers to an excipient whose primary function is to modify the duration or place of release of the active substance from a dosage form as compared with a conventional immediate release dosage form.

The term “non-release controlling excipient” refers to an excipient whose primary function do not include modifying the duration or place of release of the active substance from a dosage form as compared with a conventional immediate release dosage form.

The term “modulate” refers to the ability of a compound to increase or decrease the function, or activity, of an alpha-adrenergic receptor.

The term “prodrug” refers to an agent that is converted into the parent drug in vivo. Prodrugs are often useful because, in some situations, they may be easier to administer than the parent drug.

The terms “alkyl” and “substituted alkyl” are interchangeable and include substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (—CD₃), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group consisting of hydrogen, deuterium, halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₇₀ and —C₀₋₁₀alkylCONR₇₁R₇₂ wherein R₇₀, R₇₁ and R₇₂ are independently selected from the group consisting of hydrogen, deuterium, alkyl, aryl, or R₇₁ and R₇₂ are taken together with the nitrogen to which they are attached forming a saturated cyclic or unsaturated cyclic system containing 3 to 8 carbon atoms with at least one substituent as defined herein.

The term “alkyloxy” (e.g. methoxy, ethoxy, propyloxy, allyloxy, cyclohexyloxy) represents a substituted or unsubstituted alkyl group as defined above having the indicated number of carbon atoms attached through an oxygen bridge.

The term “aryl” and “substituted aryl” are interchangeable and include substituted, optionally substituted and unsubstituted, monocyclic, polycyclic, biaryl aromatic groups covalently attached at any ring position capable of forming a stable covalent bond, certain preferred points of attachment being apparent to those skilled in the art (e.g., 3-phenyl, 4-naphthyl and the like). The aryl substituents are independently selected from the group consisting of hydrogen, deuterium, halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₇₀, and —C₀₋₁₀alkylCONR₇₁R₇₂ wherein R₇₀, R₇₁ and R₇₂ are independently selected from the group consisting of hydrogen, deuterium, alkyl, aryl or R₇₁ and R₇₂ are taken together with the nitrogen to which they are attached forming a saturated cyclic or unsaturated cyclic system containing 3 to 8 carbon atoms with at least one substituent as defined above.

The definition of “aryl” includes but is not limited to phenyl, pentadeuterophenyl, biphenyl, naphthyl, dihydronaphthyl, tetrahydronaphthyl, indenyl, indanyl, azulenyl, anthryl, phenanthryl, fluorenyl, pyrenyl and the like.

In light of the purposes described in the present disclosure, all references to “alkyl” and “aryl” groups or any groups ordinarily containing C—H bonds may include partially or fully deuterated versions as required to affect the improvements outlined herein.

### Deuterium Kinetic Isotope Effect

In an attempt to eliminate foreign substances, such as therapeutic agents, from its circulation system, the animal body expresses various enzymes, such as the cytochrome P₄₅₀ enzymes or CYPs, esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}, where E_{act} is the activation energy, T is temperature, R is the molar gas constant, k is the rate constant for the reaction, and A (the frequency factor) is a constant specific to each reaction that depends on the probability that the molecules will collide with the correct orientation. The Arrhenius equation states that the fraction of molecules that have enough energy to overcome an energy barrier, that is, those with energy at least equal to the activation energy, depends exponentially on the ratio of the activation energy to thermal energy (RT), the average amount of thermal energy that molecules possess at a certain temperature.

The transition state in a reaction is a short lived state (on the order of 10⁻¹⁴ sec) along the reaction pathway during which the original bonds have stretched to their limit. By definition, the activation energy E_{act} for a reaction is the energy required to reach the transition state of that reaction. Reactions that involve multiple steps will necessarily have a number of transition states, and in these instances, the activation energy for the reaction is equal to the energy difference between the reactants and the most unstable transition state. Once the transition state is reached, the molecules can either revert, thus reforming the original reactants, or new bonds form giving rise to the products. This dichotomy is possible because both pathways, forward and reverse, result in the release of energy. A catalyst facilitates a reaction process by lowering the activation energy leading to a transition state. Enzymes are examples of biological catalysts that reduce the energy necessary to achieve a particular transition state.

A carbon-hydrogen bond is by nature a covalent chemical bond. Such a bond forms when two atoms of similar electronegativity share some of their valence electrons, thereby creating a force that holds the atoms together. This force or bond strength can be quantified and is expressed in units of energy, and as such, covalent bonds between various atoms can be classified according to how much energy must be applied to the bond in order to break the bond or separate the two atoms.

The bond strength is directly proportional to the absolute value of the ground-state vibrational energy of the bond. This vibrational energy, which is also known as the zero-point vibrational energy, depends on the mass of the atoms that form the bond. The absolute value of the zero-point vibrational energy increases as the mass of one or both of the atoms making the bond increases. Since deuterium (D) has twice the mass of hydrogen (H), it follows that a C-D bond is stronger than the corresponding C—H bond. Compounds with C-D bonds are frequently indefinitely stable in H₂O, and have been widely used for isotopic studies. If a C—H bond is broken during a rate-determining step in a chemical reaction (i.e. the step with the highest transition state energy), then substituting a deuterium for that hydrogen will cause a decrease in the reaction rate and the process will slow down. This phenomenon is known as the Deuterium Kinetic Isotope Effect (DKIE) and can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small size of a hydrogen atom, and occurs because transition states involving a proton can sometimes form in the absence of the required activation energy. A deuterium is larger and statistically has a much lower probability of undergoing this phenomenon. Substitution of tritium for hydrogen results in yet a stronger bond than deuterium and gives numerically larger isotope effects.

Discovered in 1932 by Urey, deuterium (D) is a stable and non-radioactive isotope of hydrogen. It was the first isotope to be separated from its element in pure form and has twice the mass of hydrogen, and makes up about 0.02% of the total mass of hydrogen (in this usage meaning all hydrogen isotopes) on earth. When two deuterium atoms bond with one oxygen, deuterium oxide (D₂O or “heavy water”) is formed. D₂O looks and tastes like H₂O, but has different physical properties. It boils at 101.41° C. and freezes at 3.79° C. Its heat capacity, heat of fusion, heat of vaporization, and entropy are all higher than H₂O. It is more viscous and has different solubilizing properties than H₂O.

When pure D₂O is given to rodents, it is readily absorbed and reaches an equilibrium level that is usually about eighty percent of the concentration that is consumed by the animals. The quantity of deuterium required to induce toxicity is extremely high. When 0% to as much as 15% of the body water has been replaced by D₂O, animals are healthy but are unable to gain weight as fast. as the control (untreated) group. When about 15% to about 20% of the body water has been replaced with D₂O, the animals become excitable. When about 20% to about 25% of the body water has been replaced with D₂O, the animals are so excitable that they go into frequent convulsions when stimulated. Skin lesions, ulcers on the paws and muzzles, and necrosis of the tails appear. The animals also become very aggressive; males becoming almost unmanageable. When about 30%, of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Tritium (T) is a radioactive isotope of hydrogen, used in research, fusion reactors, neutron generators and radiopharmaceuticals. Mixing tritium with a phosphor provides a continuous light source, a technique that is commonly used in wristwatches, compasses, rifle sights and exit signs. It was discovered by Rutherford, Oliphant and Harteck in 1934, and is produced naturally in the upper atmosphere when cosmic rays react with H₂ molecules. Tritium is a hydrogen atom that has 2 neutrons in the nucleus and has an atomic weight close to 3. It occurs naturally in the environment in very low concentrations, most commonly found as T₂O, a colorless and odorless liquid. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk.

Deuteration of pharmaceuticals to improve pharmacokinetics (PK), pharmacodynamics (PD), and toxicity profiles, has been demonstrated previously with some classes of drugs. For example, DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching which may even give rise to an oxidative intermediate with a faster off-rate from an activating Phase I enzyme (e.g., cytochrome P₄₅₀ 3A4). The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity. Such pitfalls have not been heretofore sufficiently predictable a priori for any drug class.

### Deuterated Adrenergic Receptor Modulators

Doxazosin (Cardura®) is an adrenergic receptor modulator. The carbon-hydrogen bonds of doxazosin contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation produce a detectable Kinetic Isotope Effect (KIE) that could affect the pharmacokinetic, pharmacologic and/or toxicologic parameters of such alpha-adrenergic modulating agents relative to compounds having naturally occurring levels of deuterium.

Aspects of the present disclosure describe an approach to designing and synthesizing new analogs of these alpha-adrenergic modulating agents through chemical modifications and derivations of the carbon-hydrogen bonds of the modulators and/or of the chemical precursors used to synthesize said modulators.

Doxazosin may be metabolized at substituted groups on the aryl moiety. Other metabolites may also exist including C—H bond oxidation at the aromatic ring. Additionally, Doxazosin is converted in vivo by oxidative and conjugative degradation to multiple metabolites. The major metabolites from phase I metabolism include compounds that are the result of mono- and di-demethylation, oxidative deamination, and/or hydroxylation. Other metabolites result from phase II metabolism, and include the glucuronidation of the hydroxylated metabolites. The application in polypharmacy is necessarily complex and has potential for adverse events because doxazosin is metabolized in part by polymorphically expressed isozymes of cytochrome P₄₅₀. This phenomenon increases inter-patient variability in response to polypharmacy. The activity of doxazosin is cut short primarily by oxidative demethylation that leads to “inactive” metabolites. These transformations may give rise to potentially reactive metabolites upon further transformation. The toxicity and pharmacology of the resultant aforementioned metabolite/s are not known with certainty but oxidation of C—H may lead to the formation of reactive metabolites which can be toxic. Limiting the production of such metabolites has the potential to decrease the danger of the administration of such drugs and may even allow increased dosage and concomitant increased efficacy. Various deuteration patterns can be used to a) reduce or eliminate unwanted metabolites, b) increase the half-life of the parent drug, c) decrease the number of doses needed to achieve a desired effect, d) decrease the amount of a dose needed to achieve a desired effect, e) increase the formation of active metabolites, if any are formed, and/or f) decrease the production of deleterious metabolites in specific tissues and/or create a more effective drug and/or a safer drug for polypharmacy, whether the polypharmacy be intentional or not. The deuteration approach has strong potential to slow the metabolism via various oxidative mechanisms.

Provided herein are compounds of Formula 1:

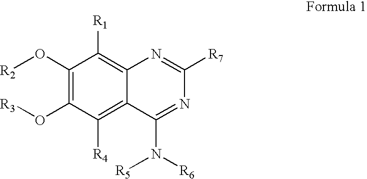

or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers, or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of:

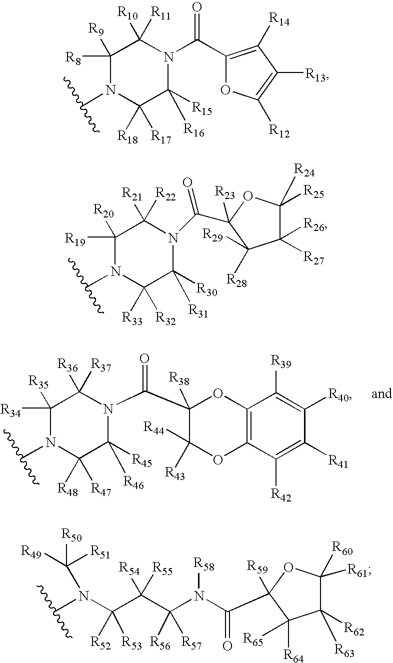

wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium; provided that compounds of Formula 1 contain at least one deuterium atom, and that deuterium enrichment in compounds of Formula 1 is at least about 1%; with the proviso that compounds of Formula 1 cannot be selected from the group consisting of:

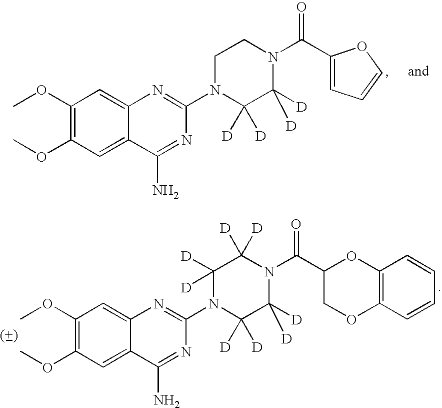

In one embodiment, the deuterium enrichment occurs at a specific position on the modulator.

In another embodiment, the deuterium enrichment is no less than about 1%.

In a further embodiment, the deuterium enrichment is no less than about 10%.

In yet a further embodiment, the deuterium enrichment is no less than about 20%.

In one embodiment, the deuterium enrichment is no less than about 50%.

In another embodiment, the deuterium enrichment is no less than about 70%.

In yet another embodiment, the deuterium enrichment is no less than about 80%.

In a further embodiment, the deuterium enrichment is no less than about 90%.

In yet a further embodiment, the deuterium enrichment is no less than about 95%.

In one embodiment, the deuterated compound has a slower rate of metabolism than the corresponding protiated quinazoline compound.

In one embodiment, the compound contains about 90% or more by weight of the (−)-enantiomer of the compound and about 10% or less by weight of (+)-enantiomer of the compound.

In another embodiment, the compound contains about 90% or more by weight of the (+)-enantiomer of the compound and about 10% or less by weight of (−)-enantiomer of the compound.

In yet another embodiment, there are provided compounds according to Formula 1 having one of the following structures:

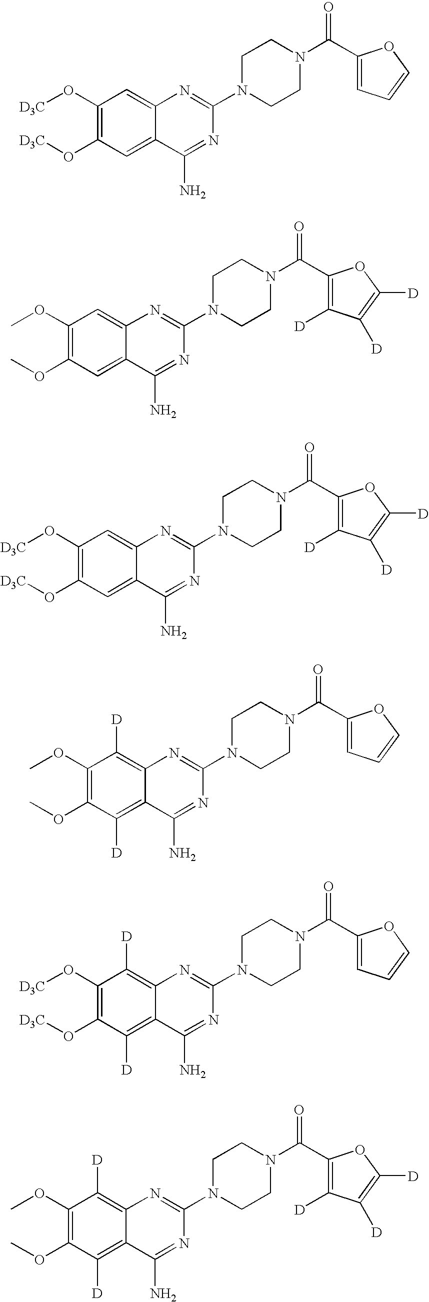

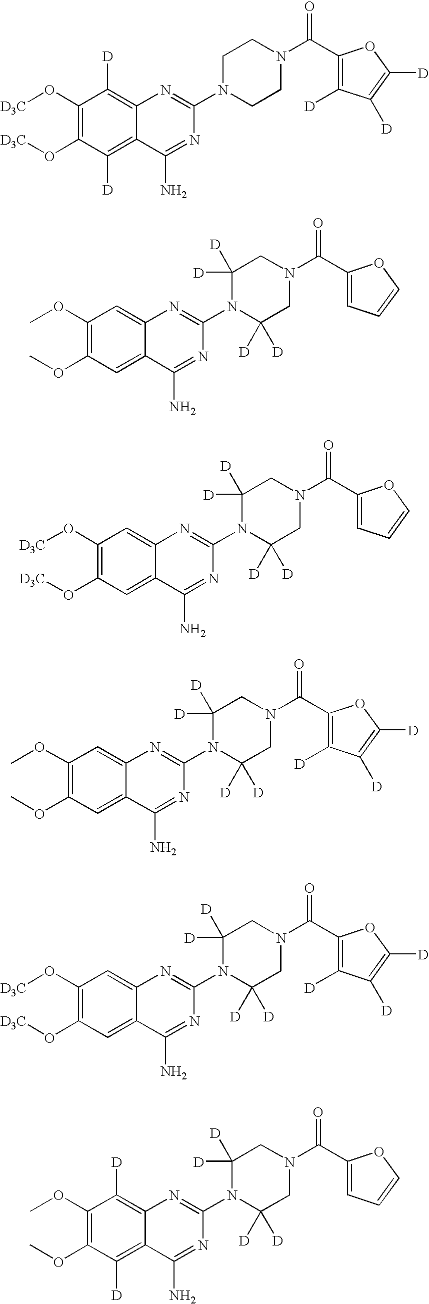

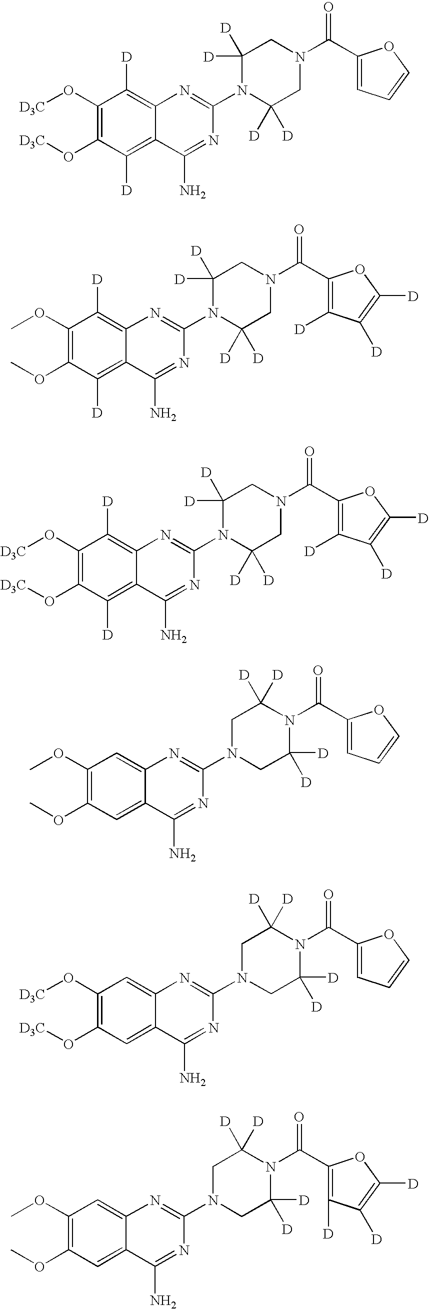

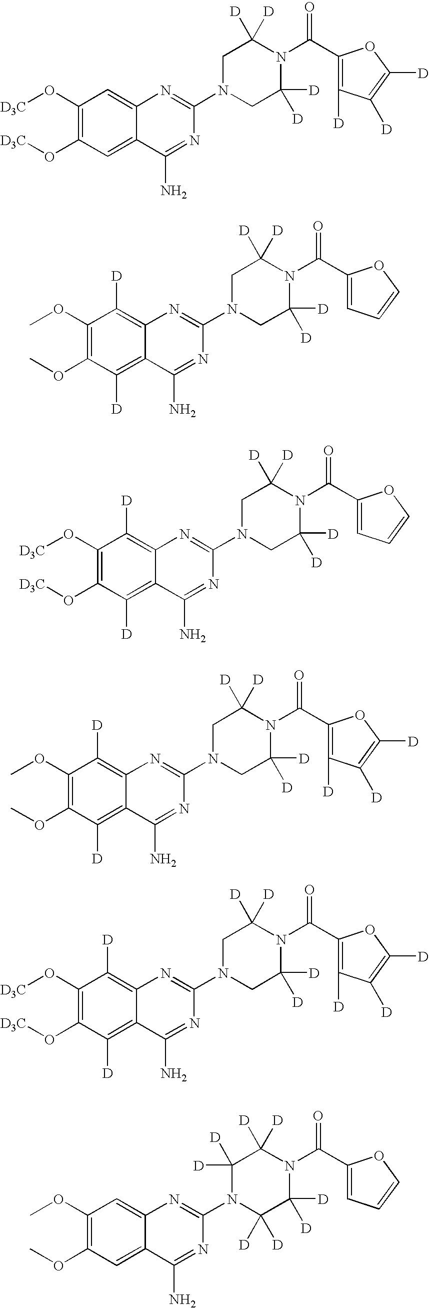

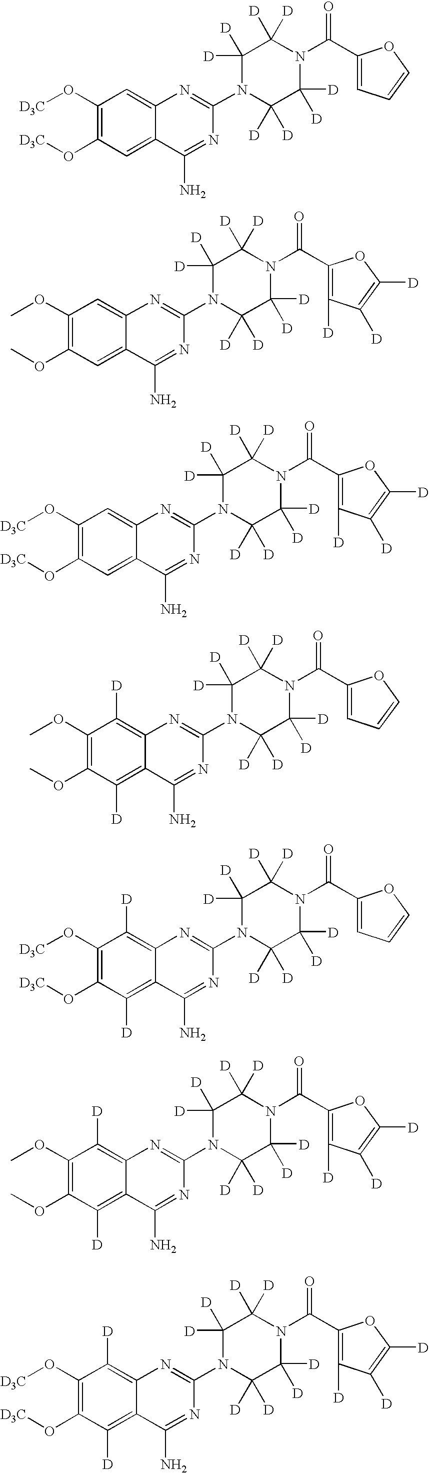

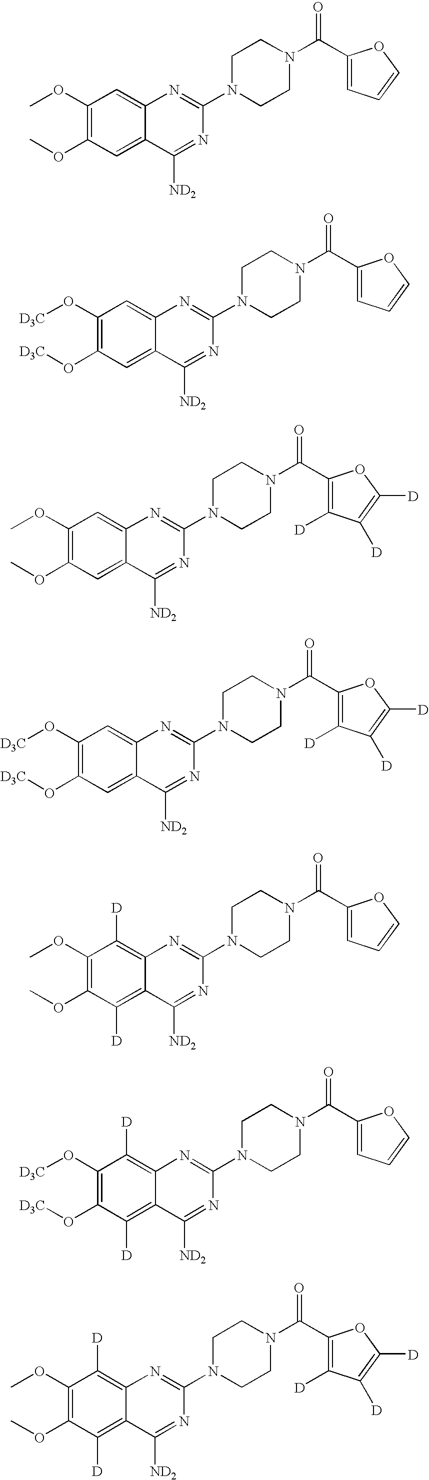

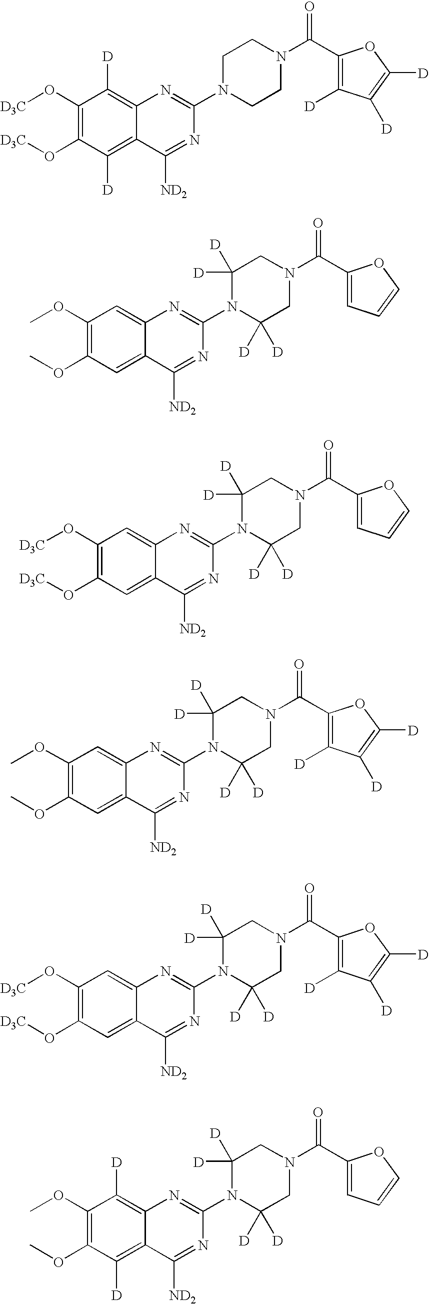

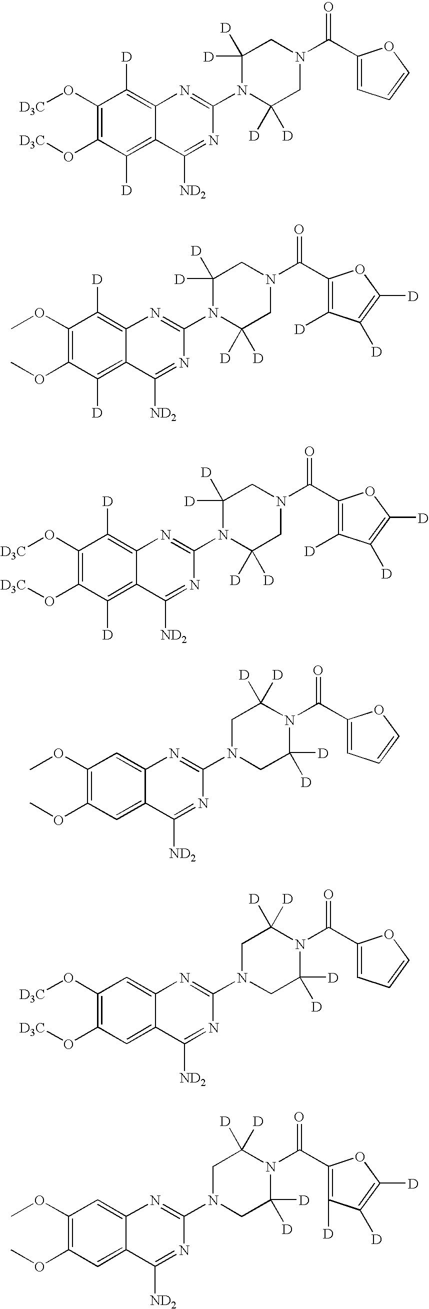

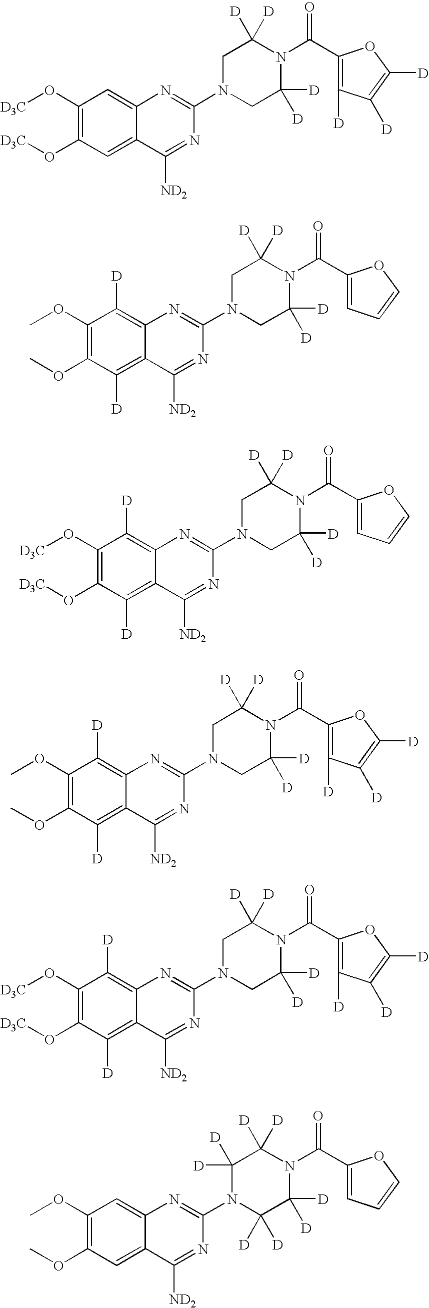

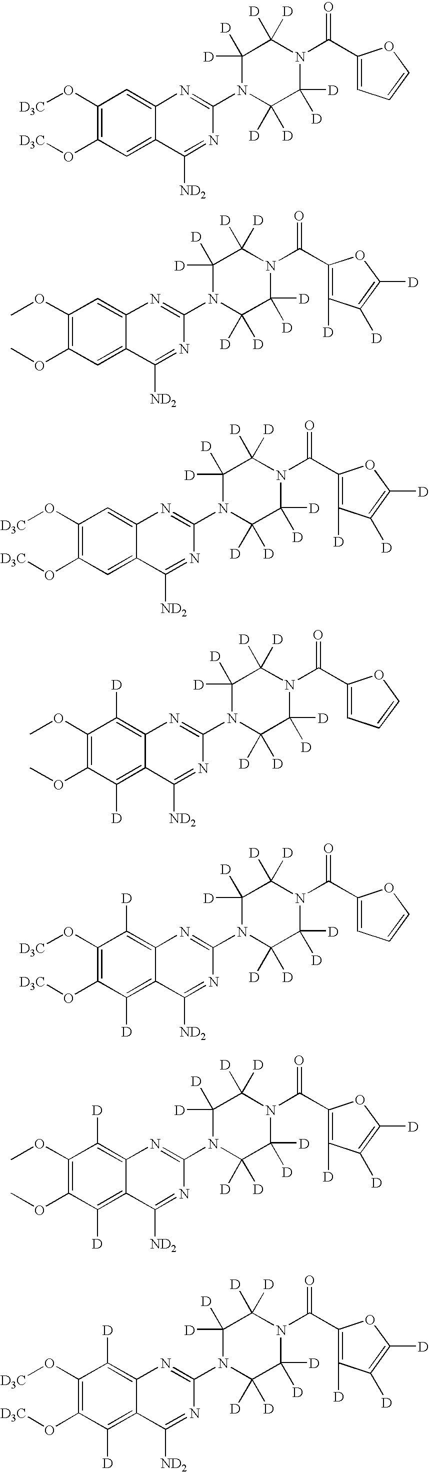

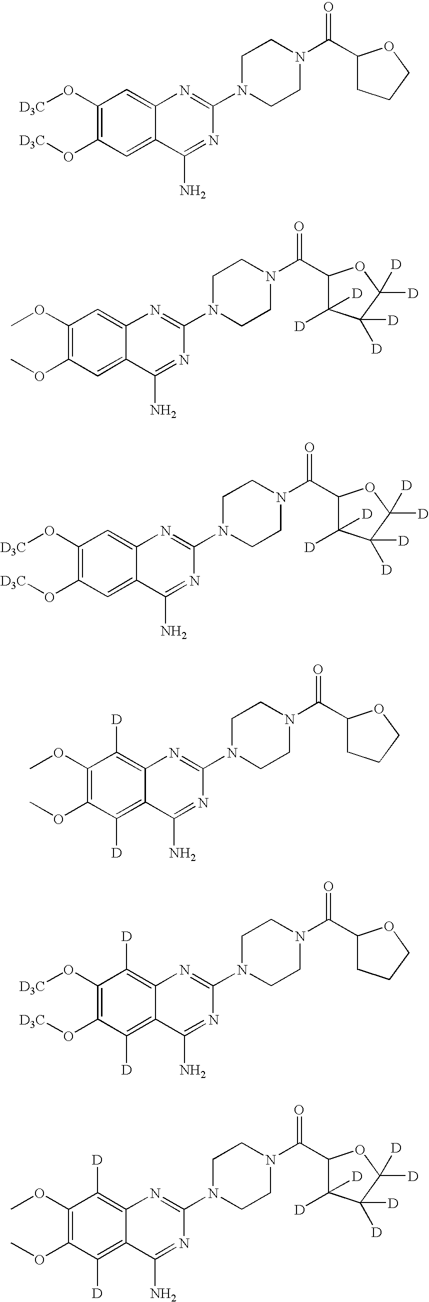

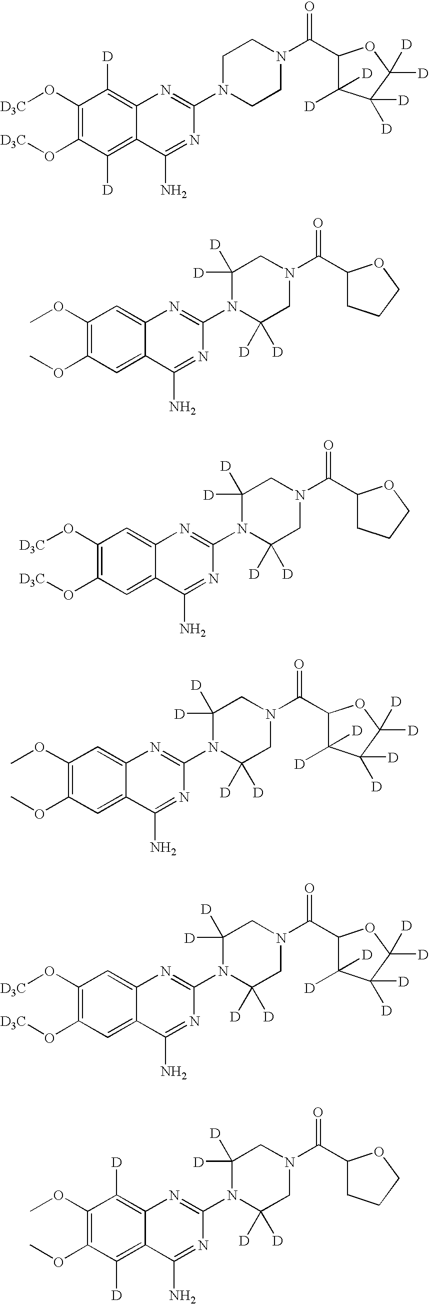

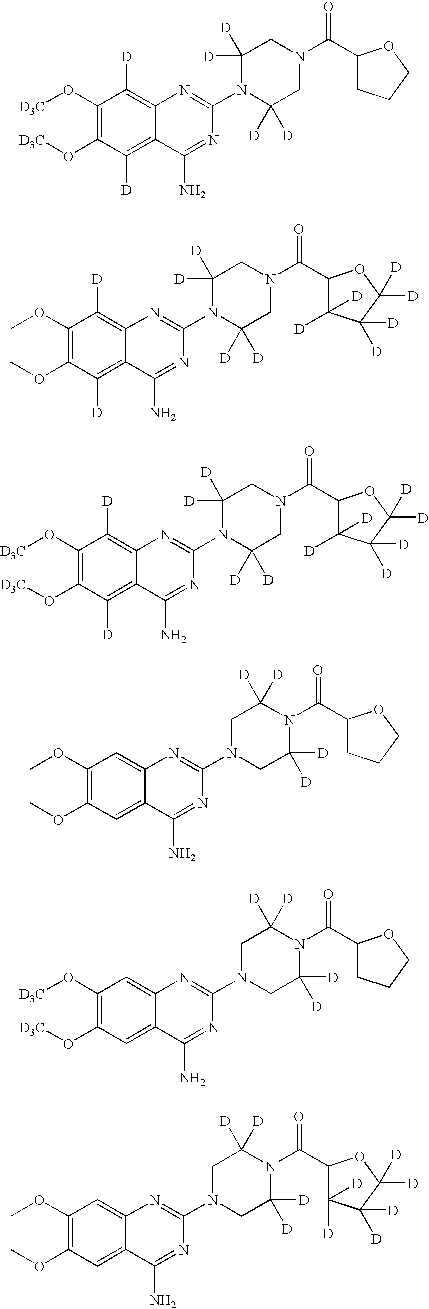

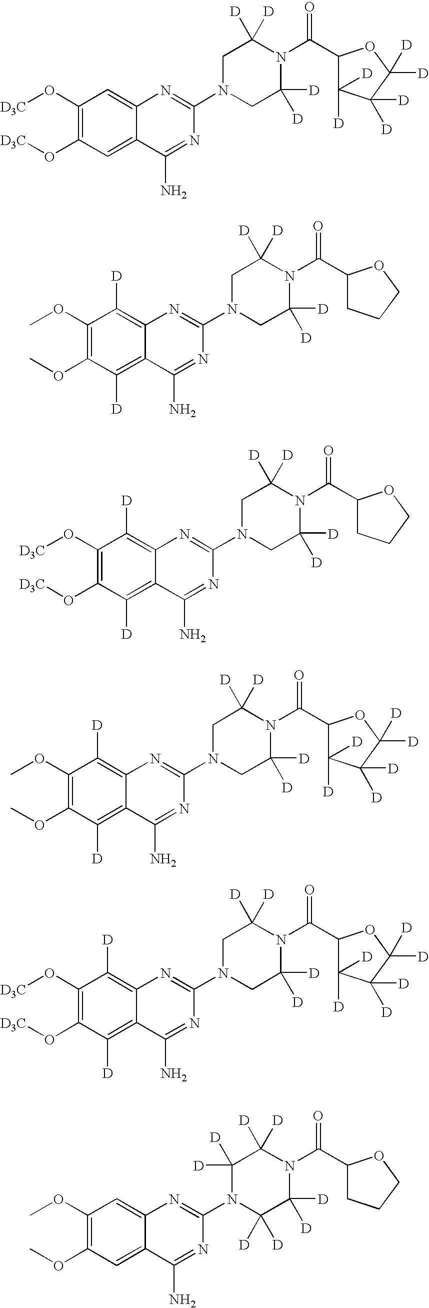

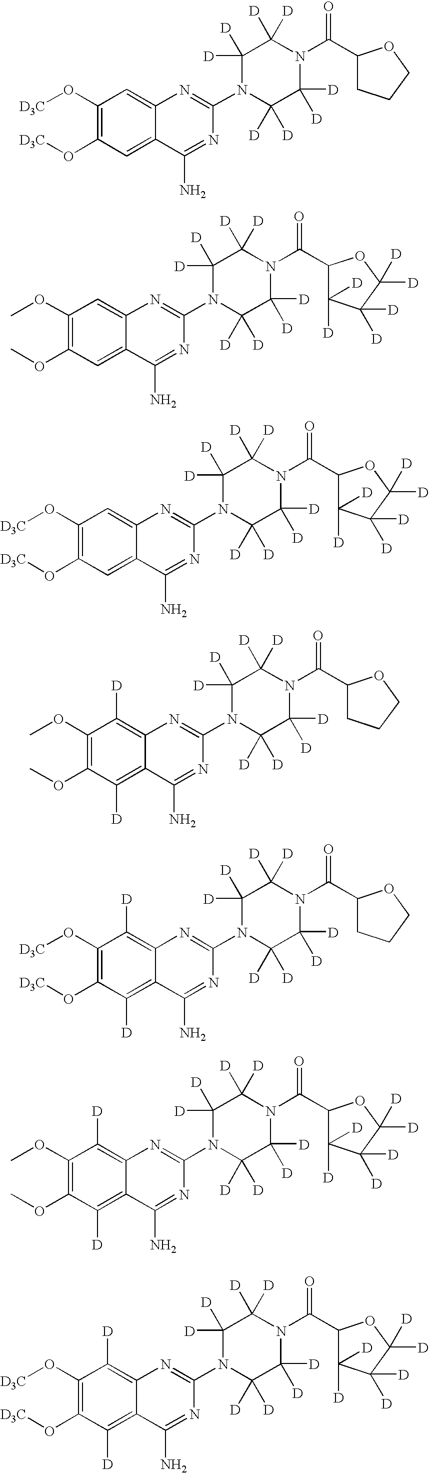

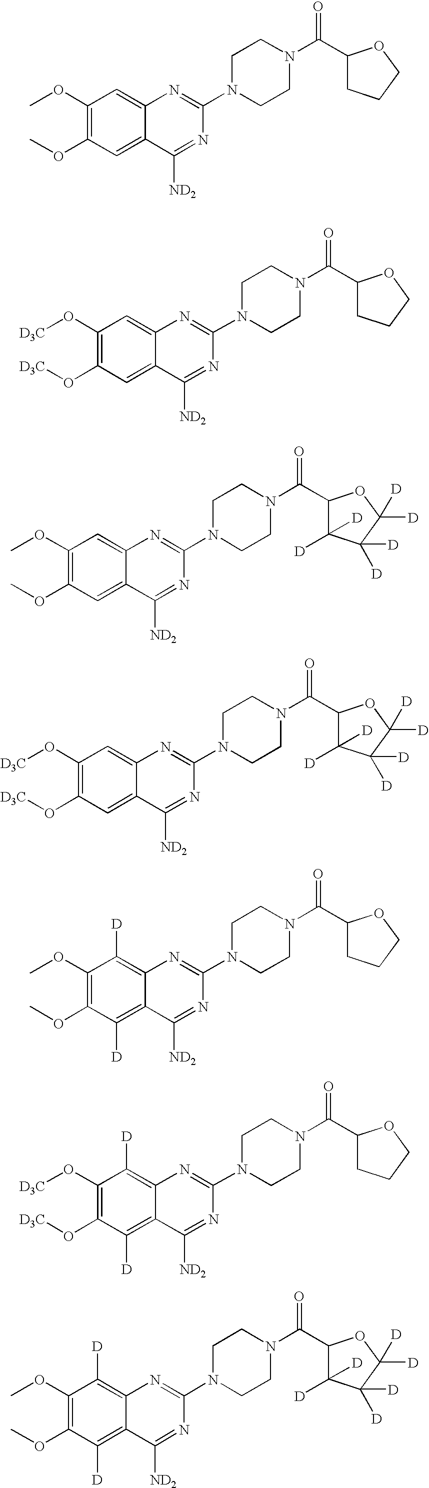

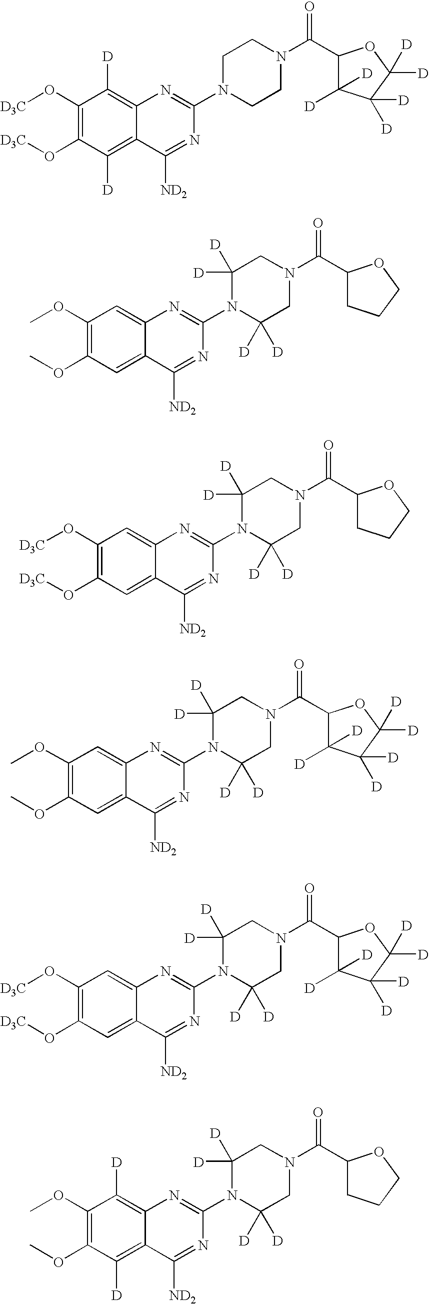

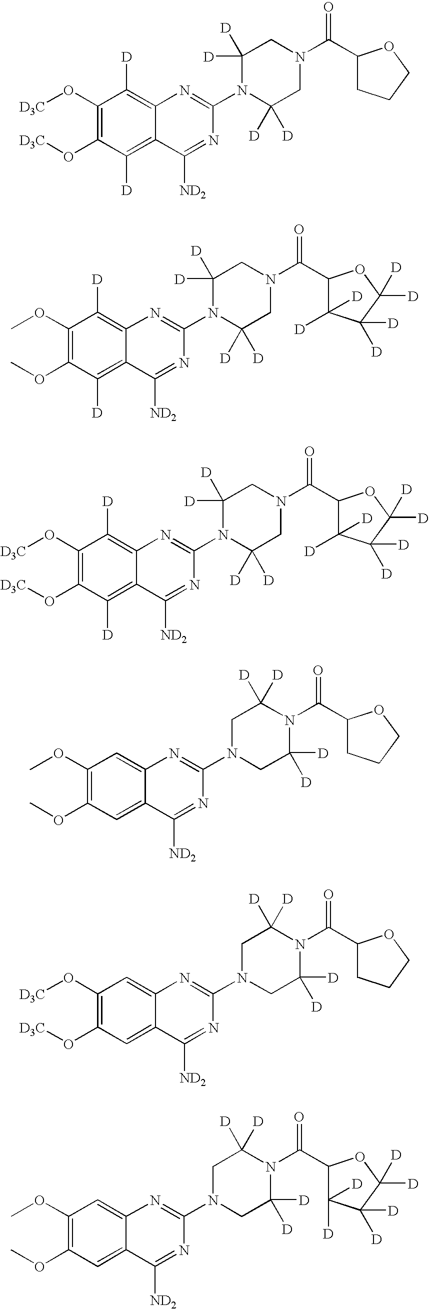

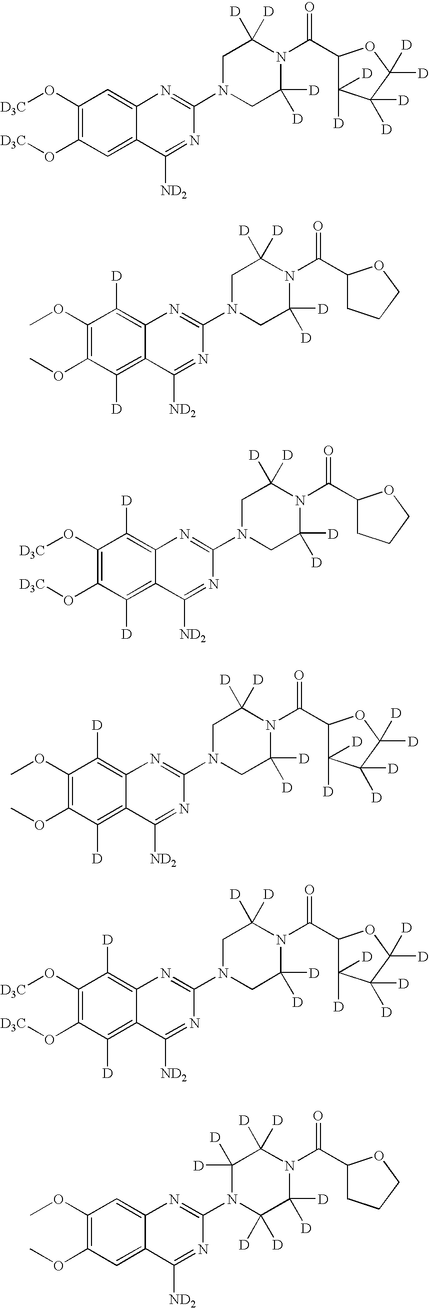

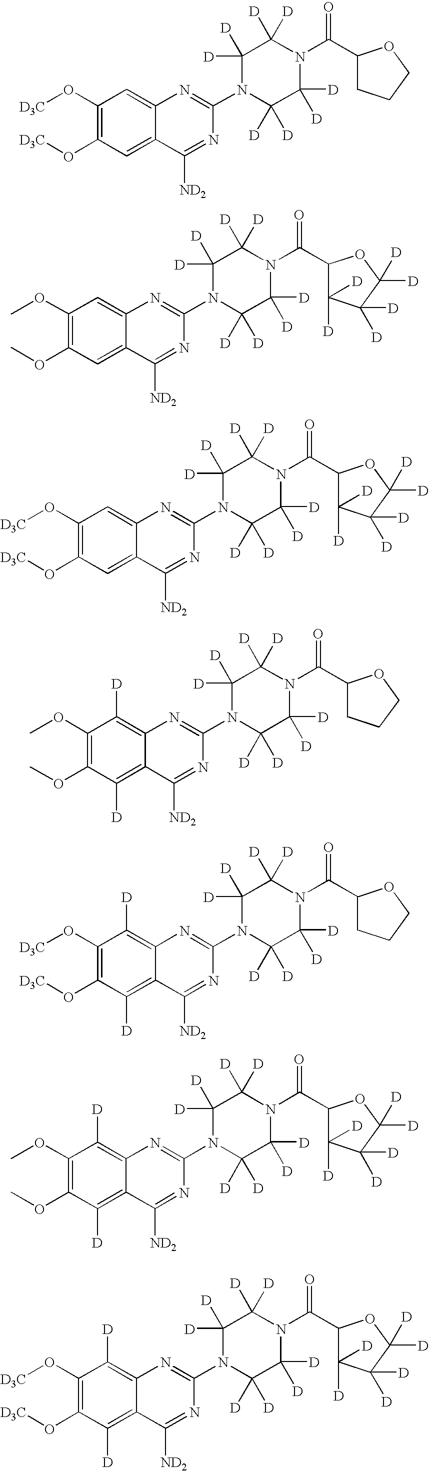

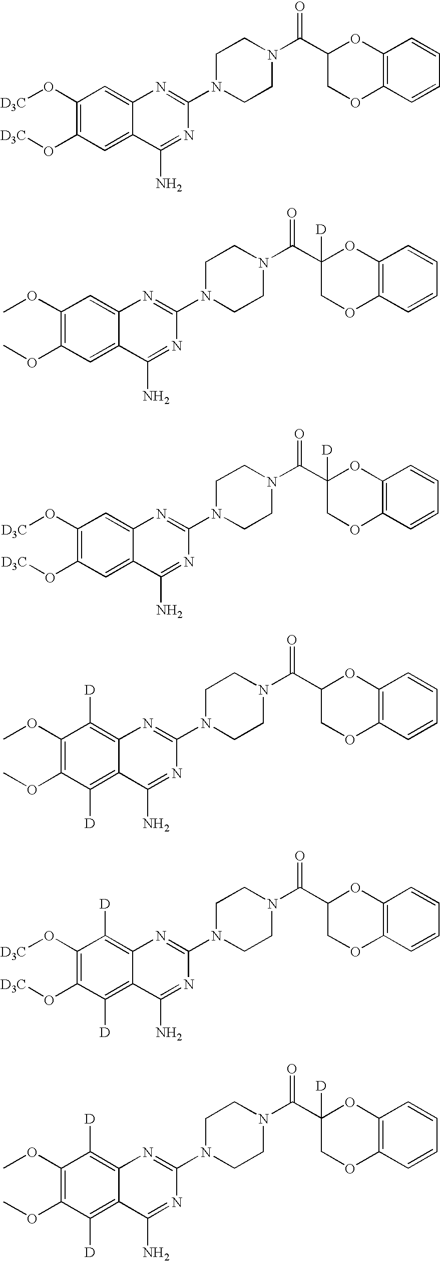

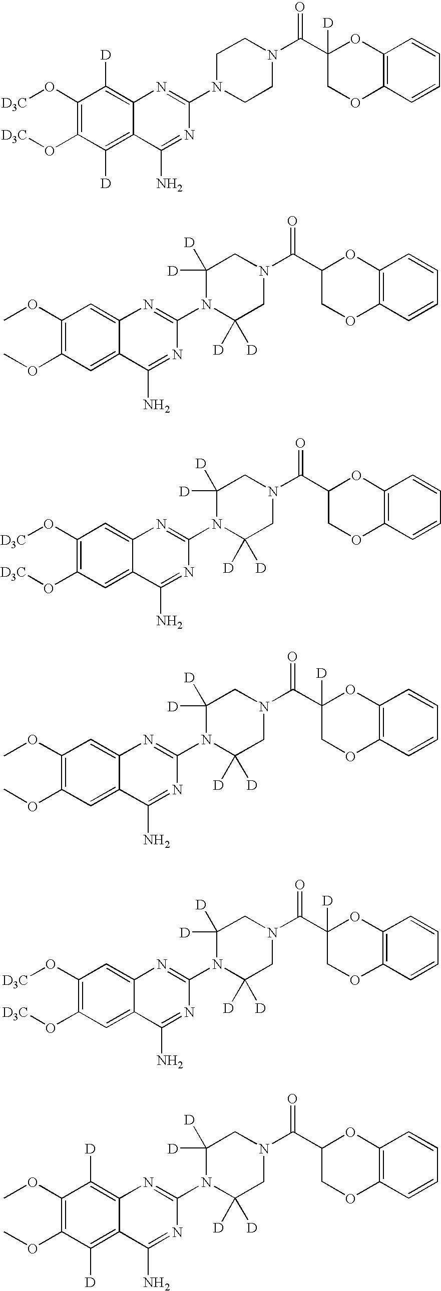

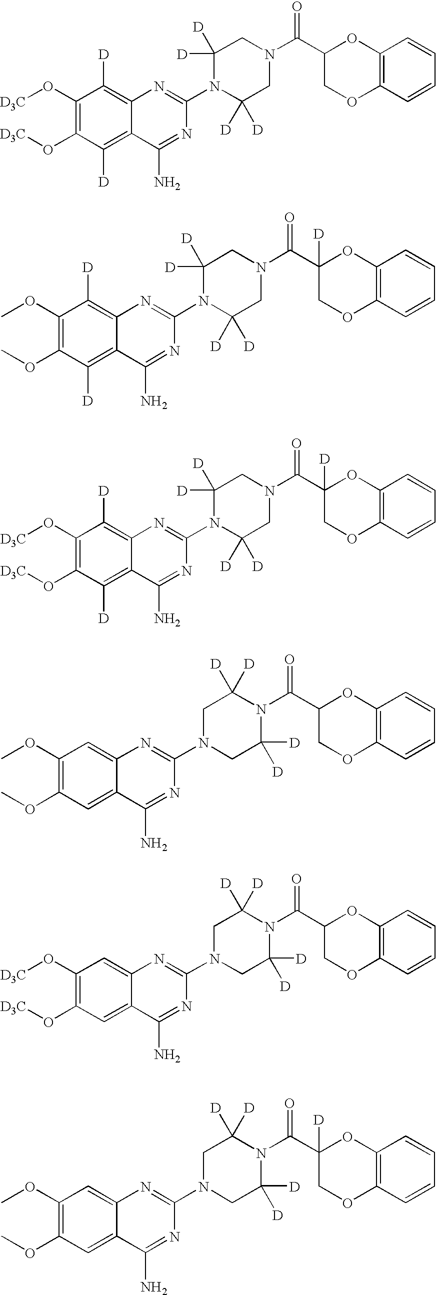

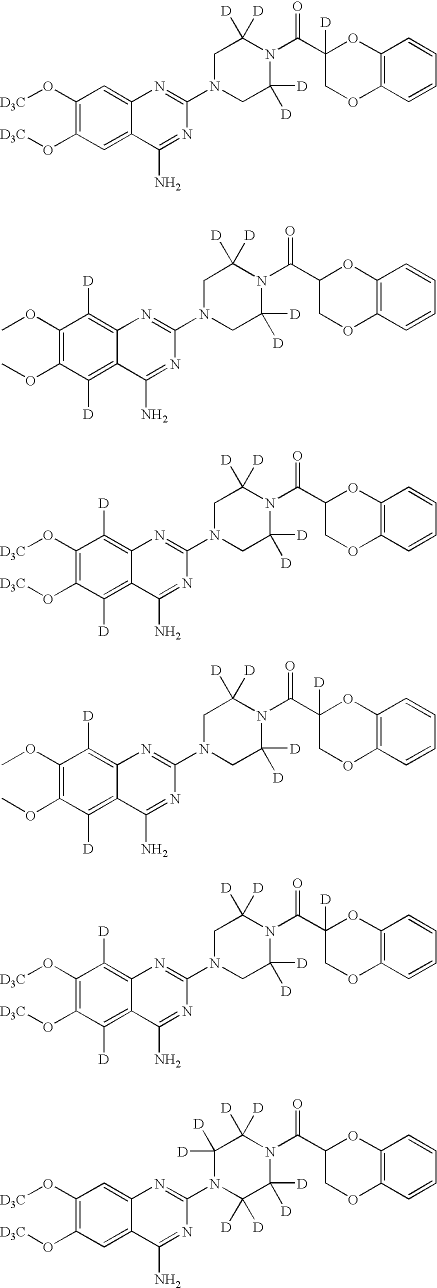

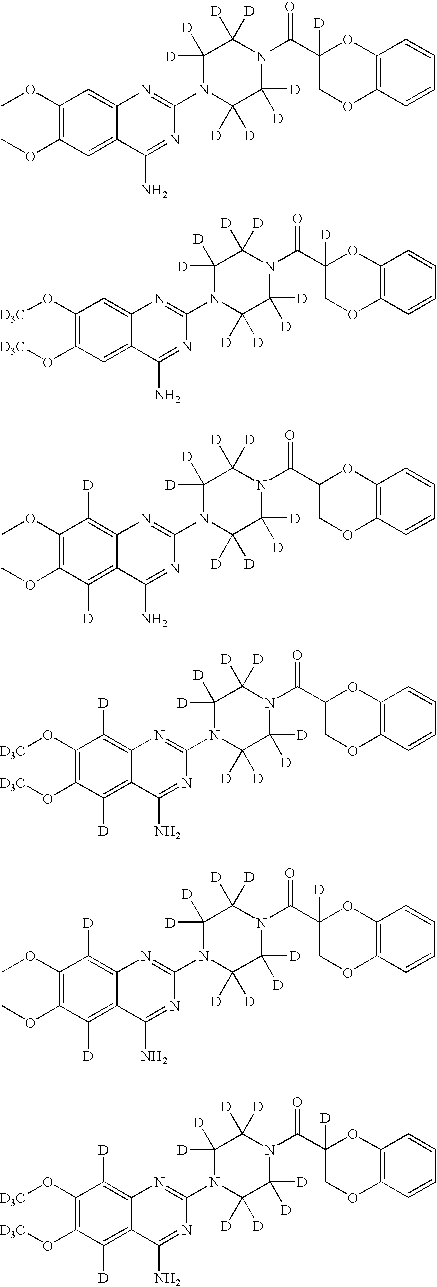

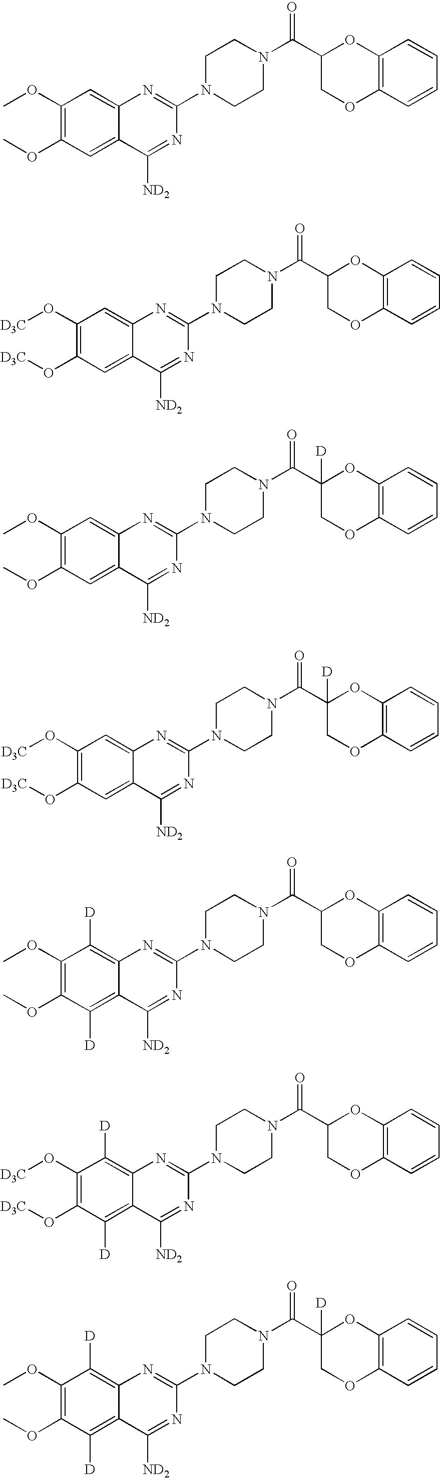

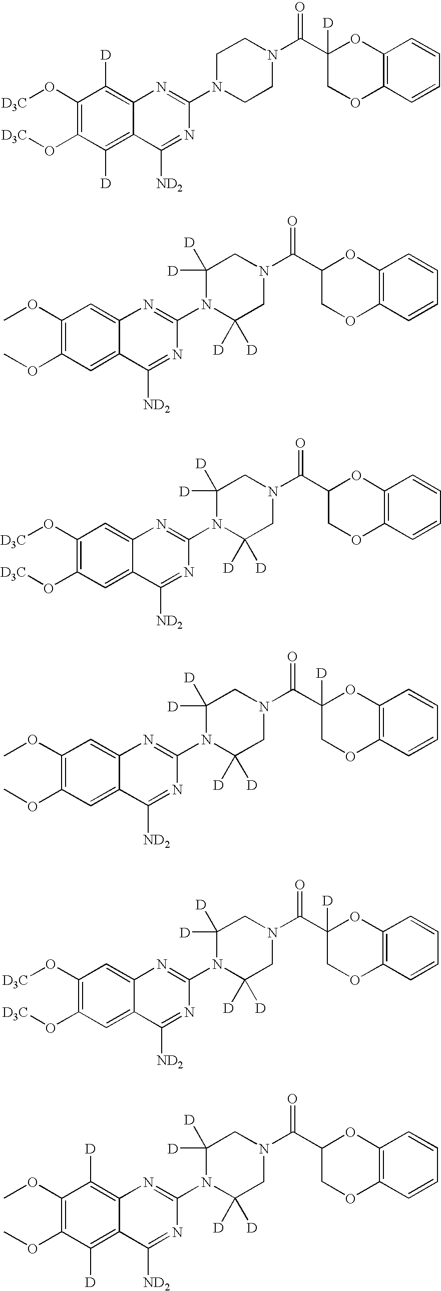

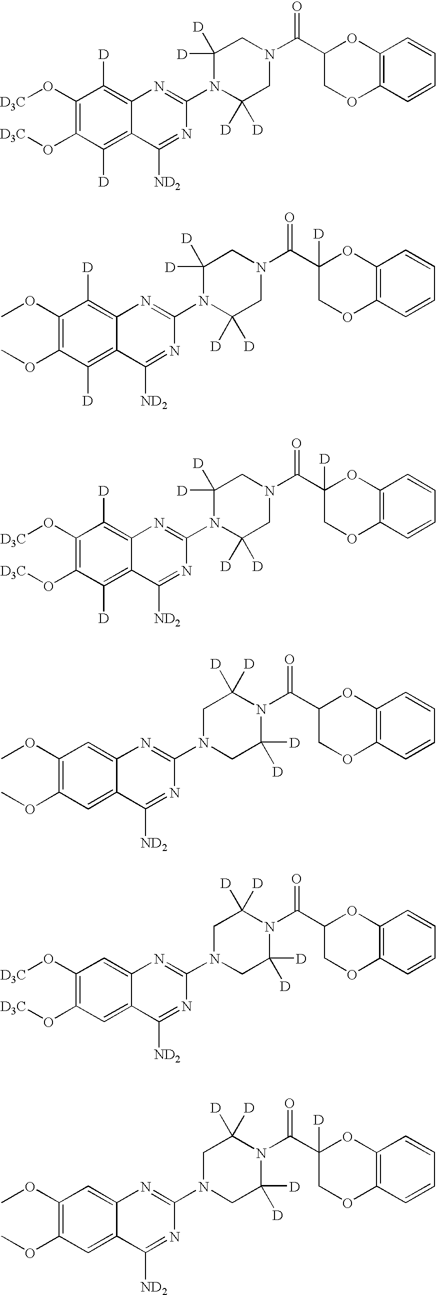

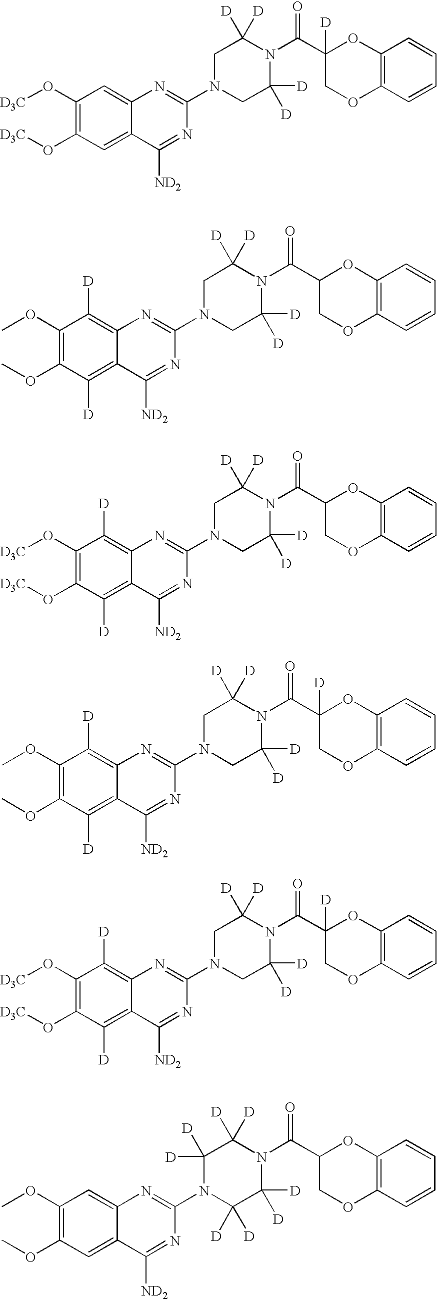

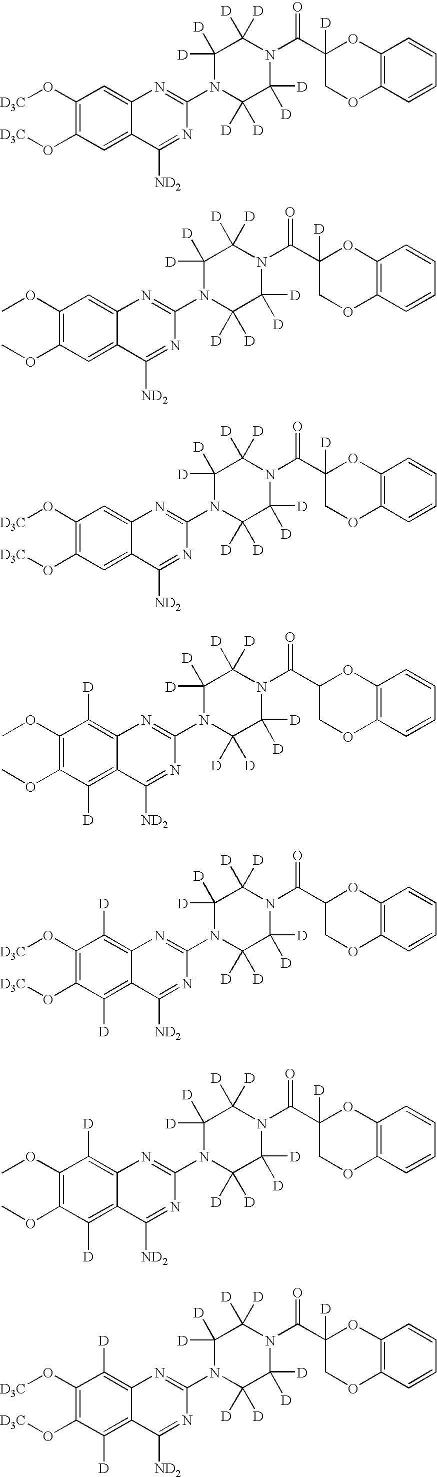

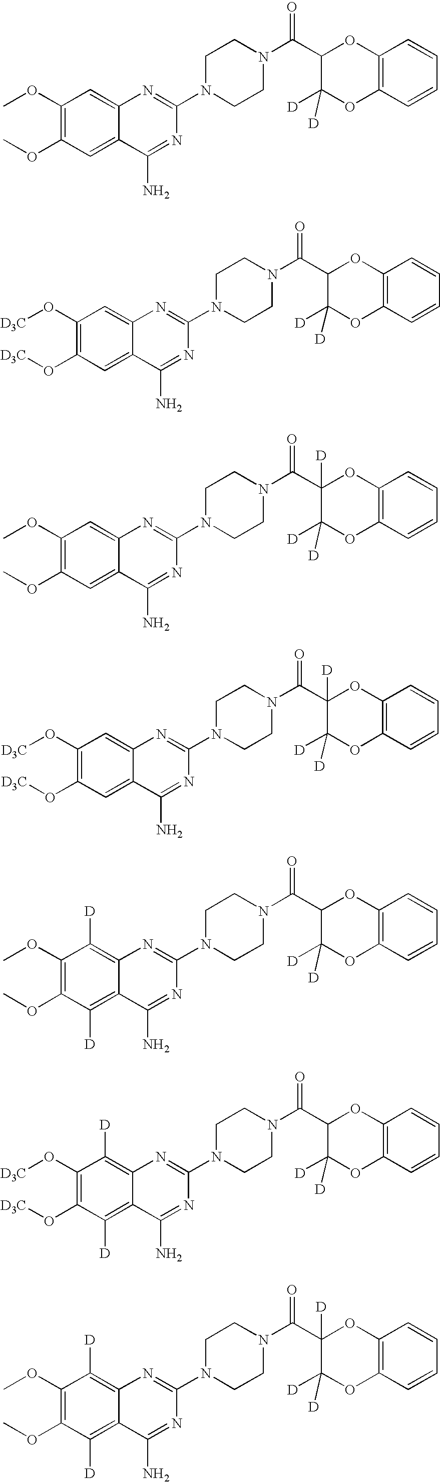

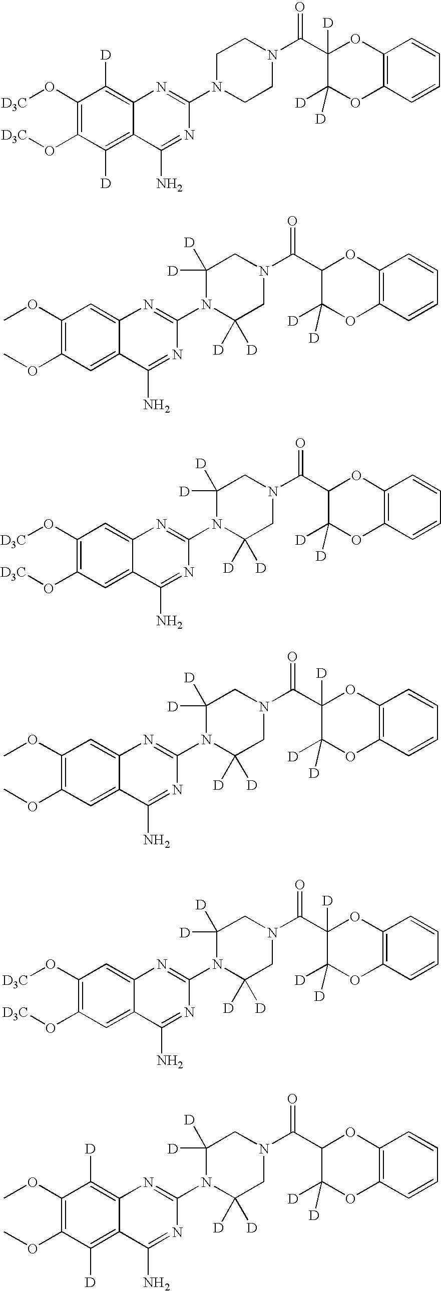

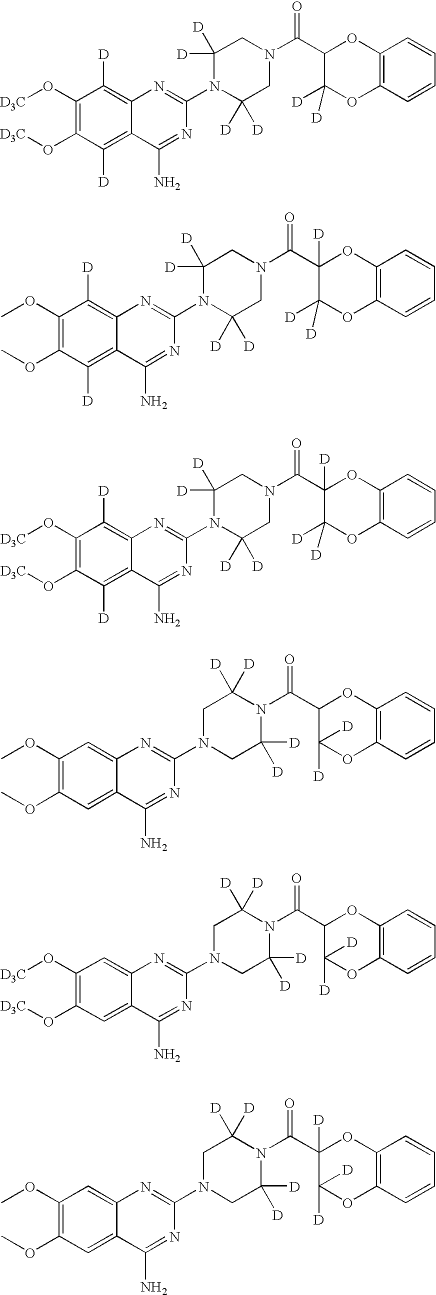

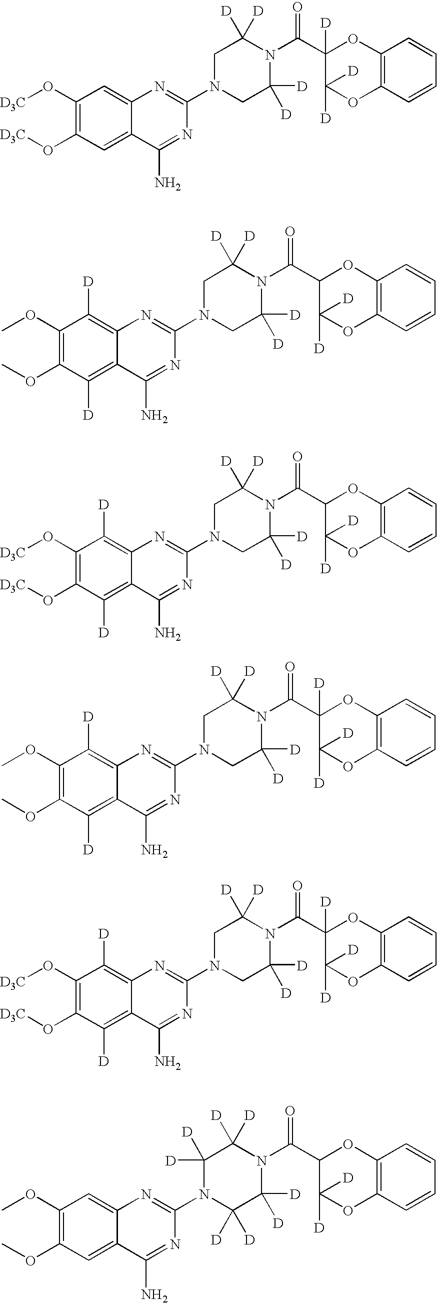

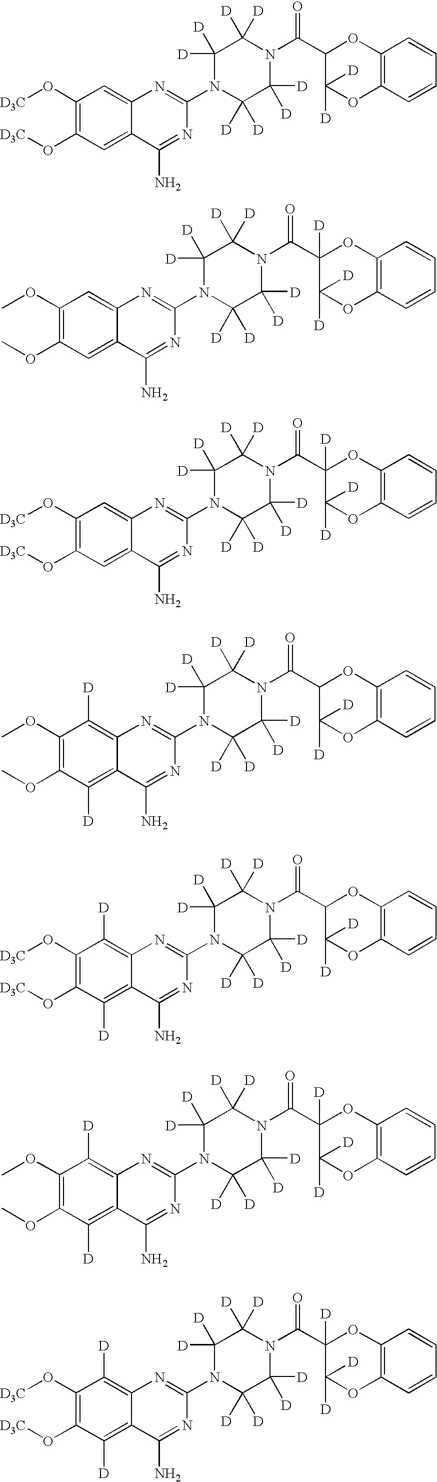

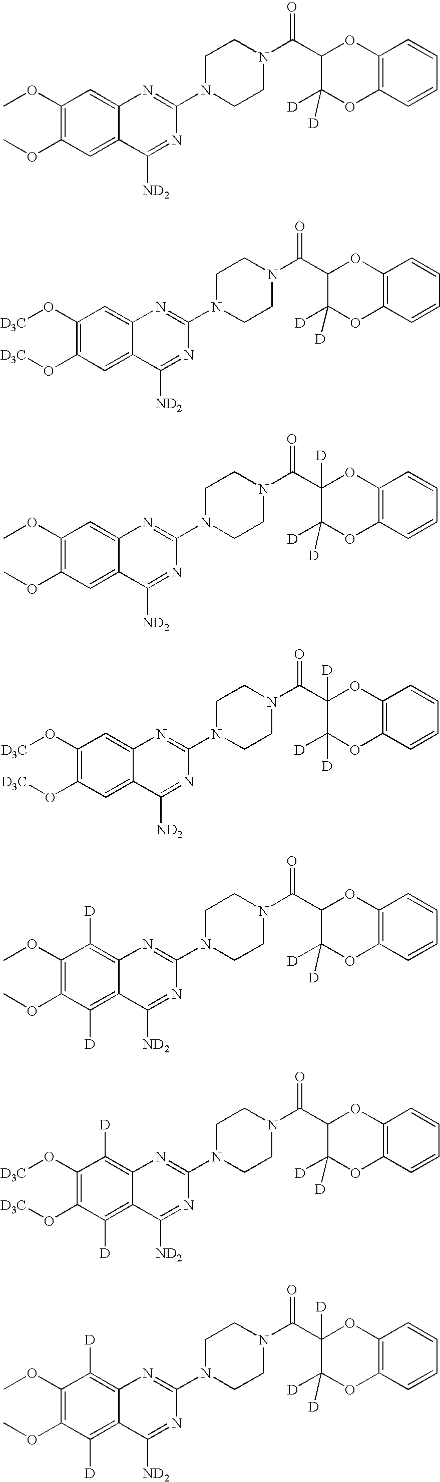

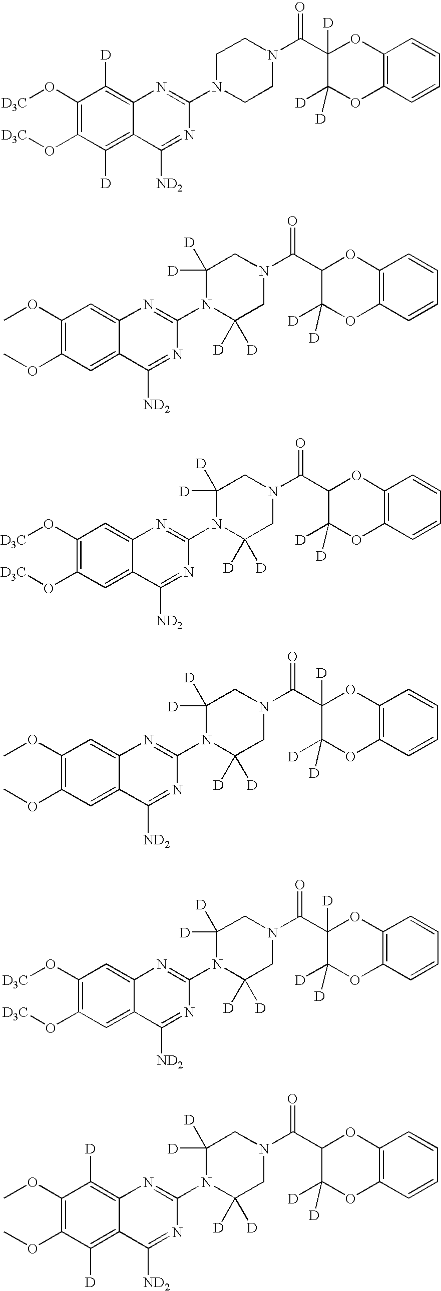

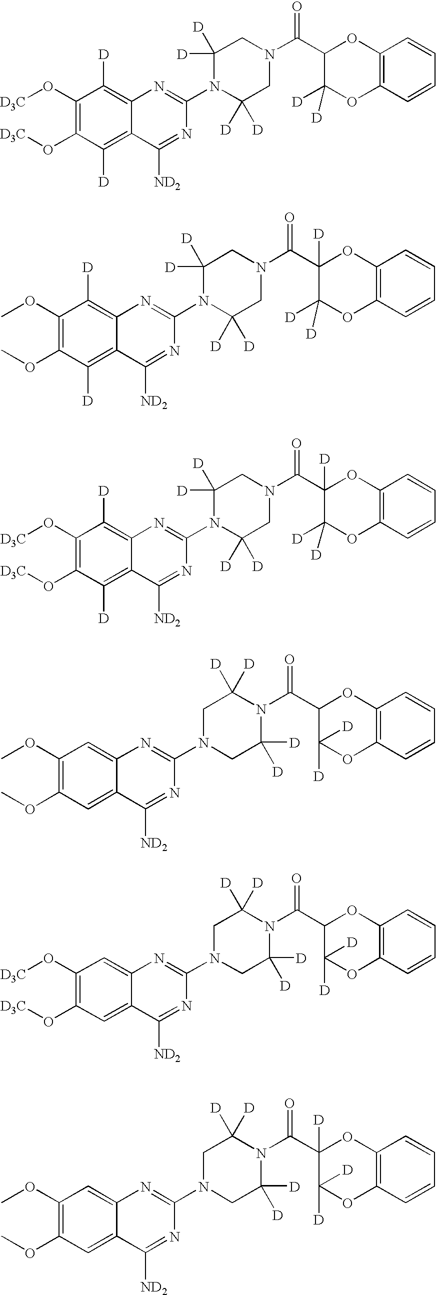

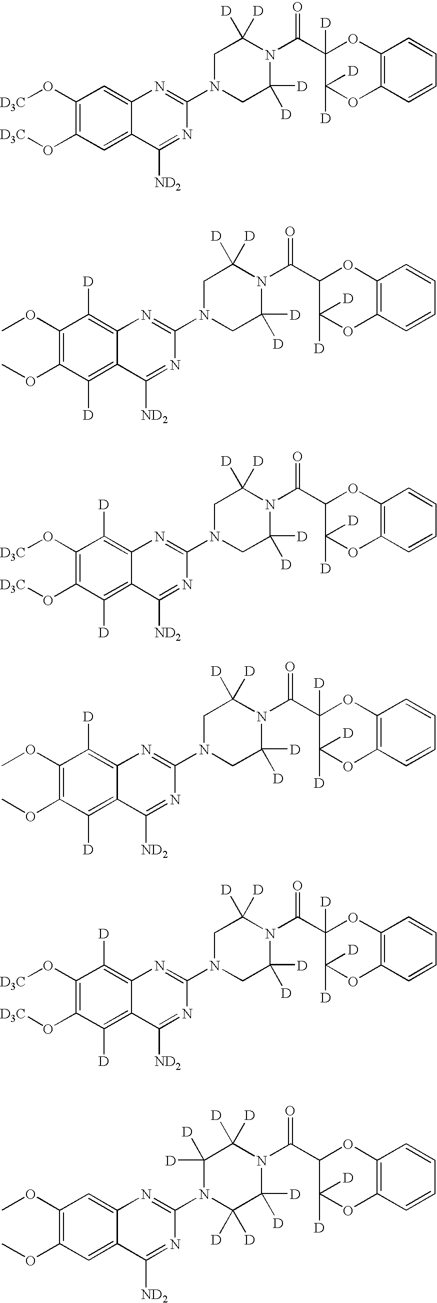

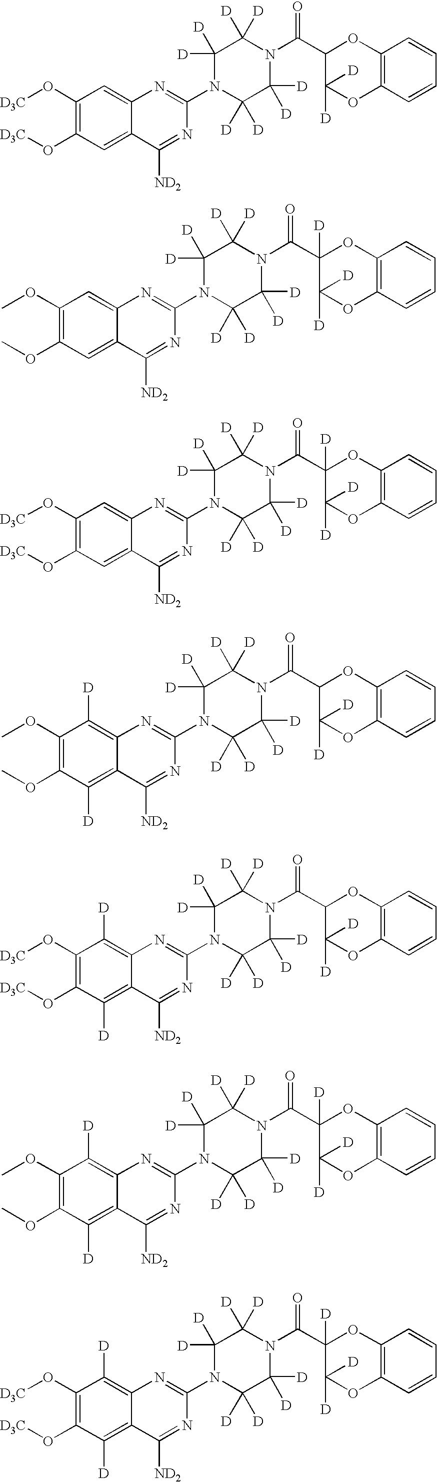

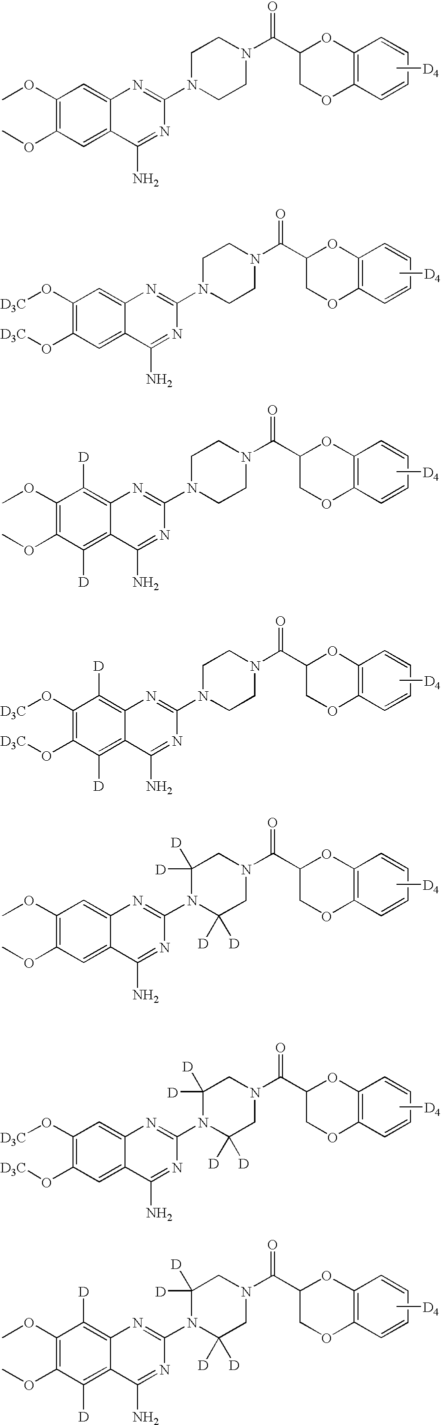

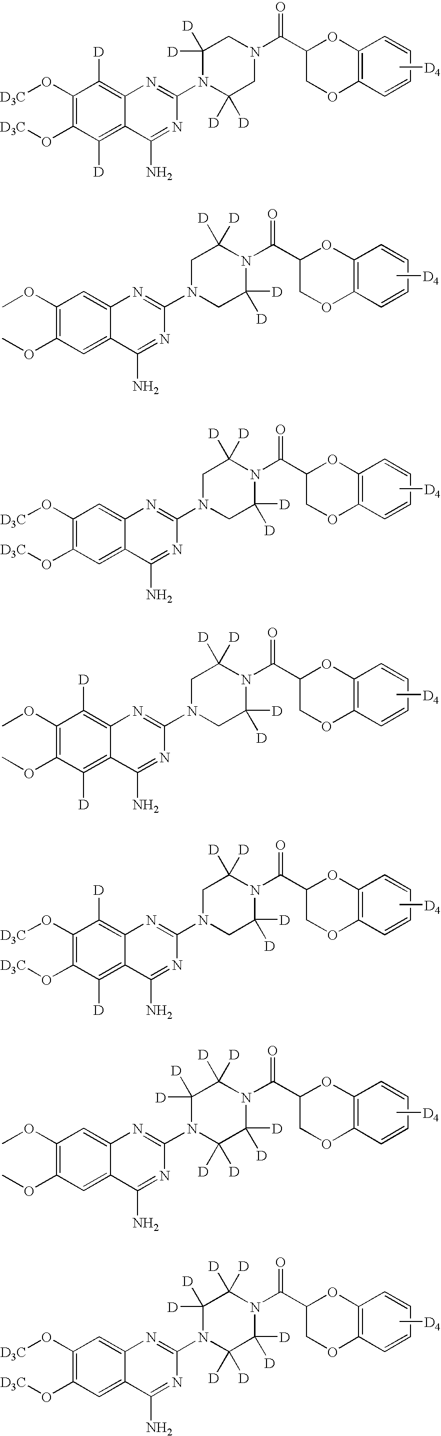

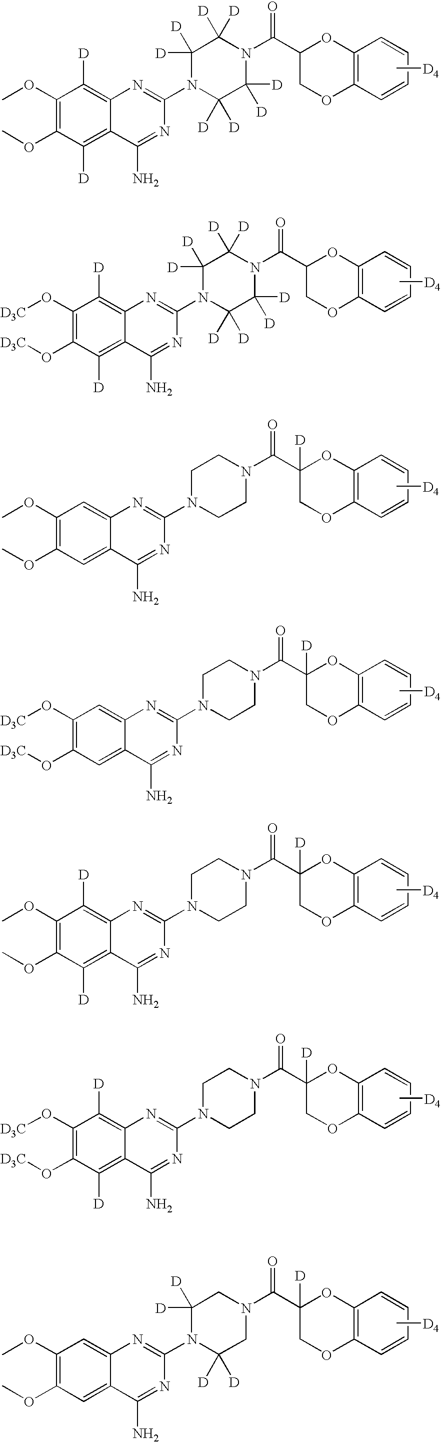

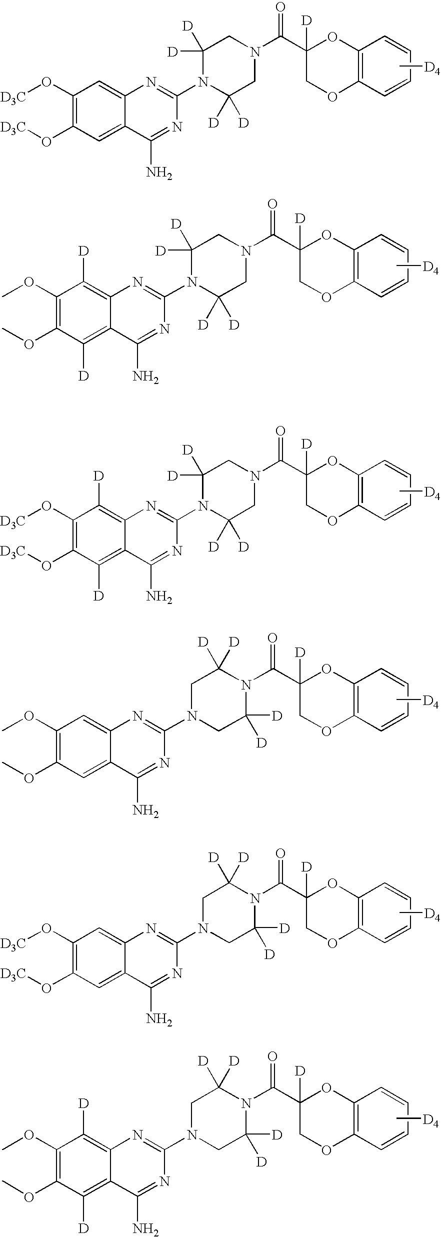

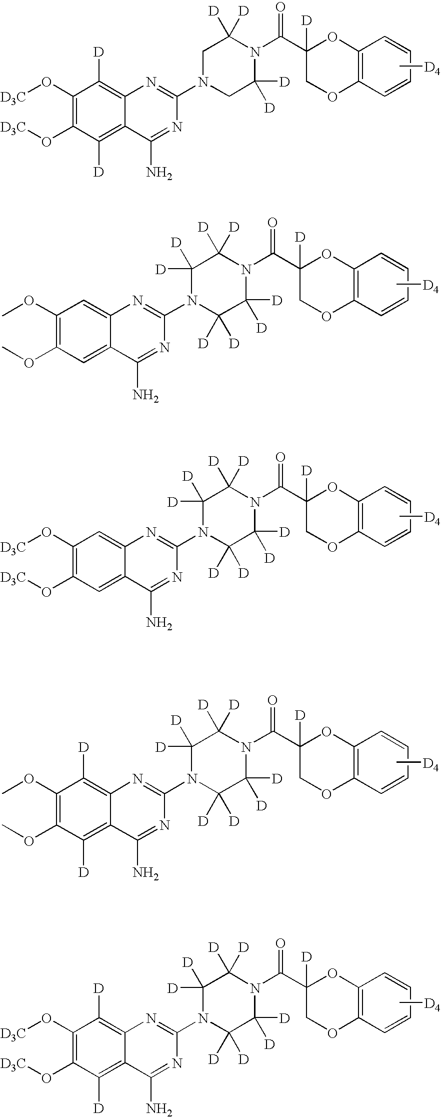

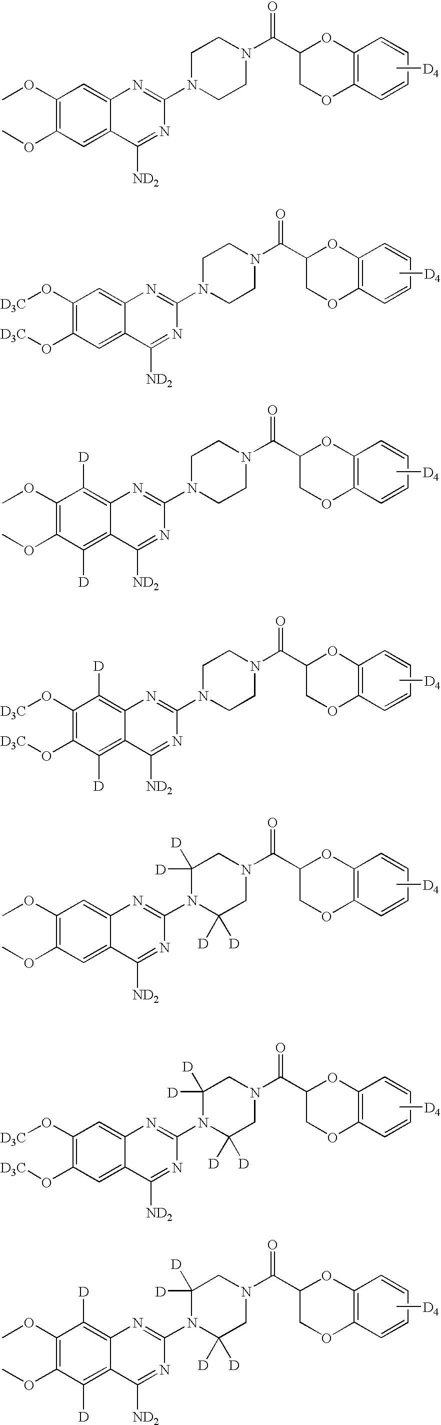

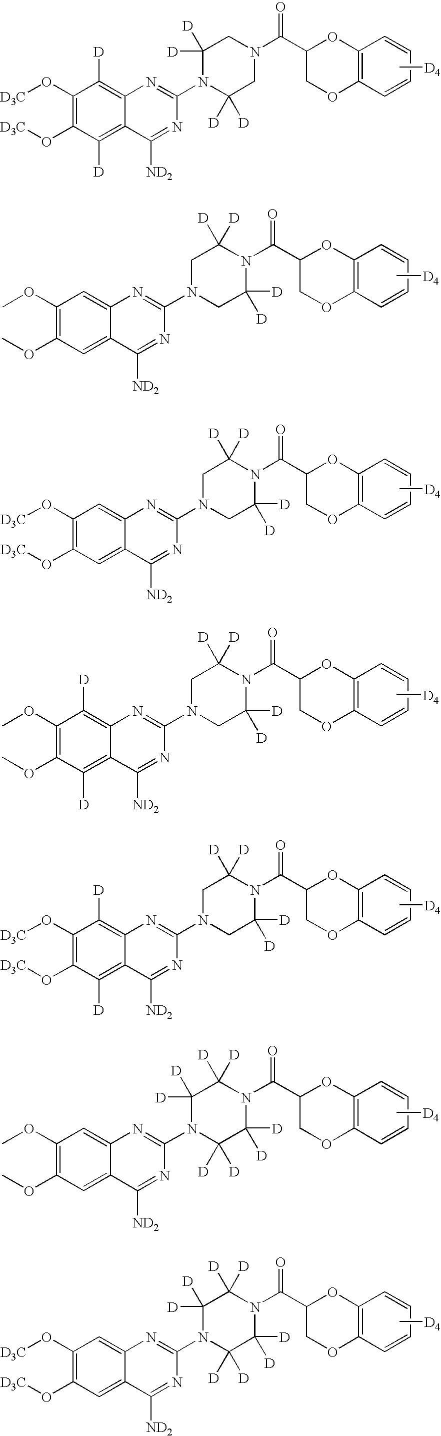

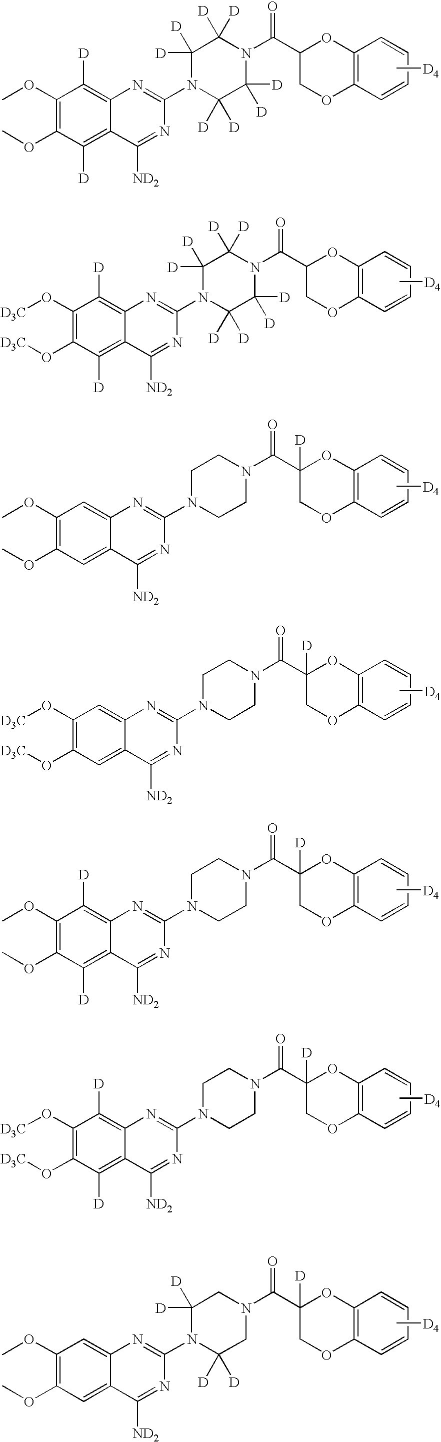

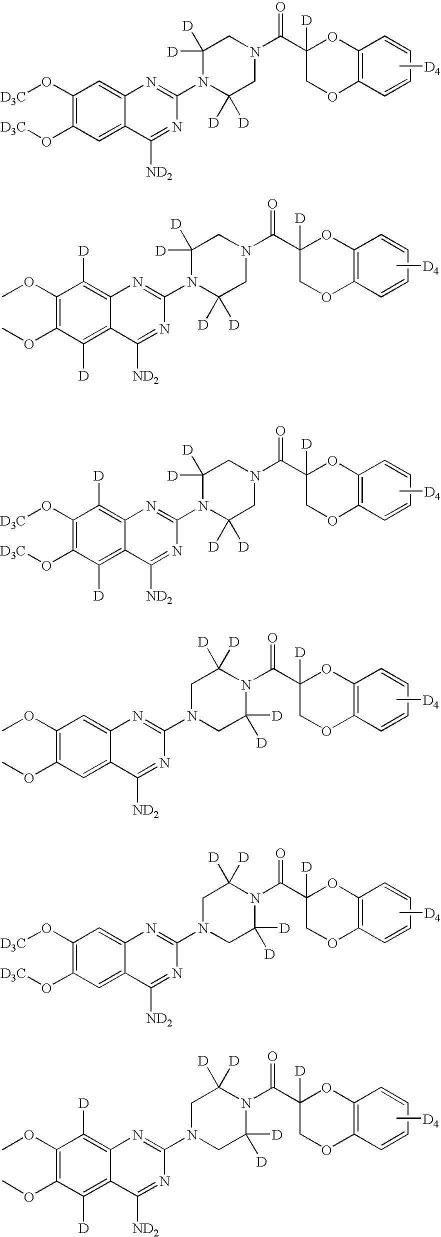

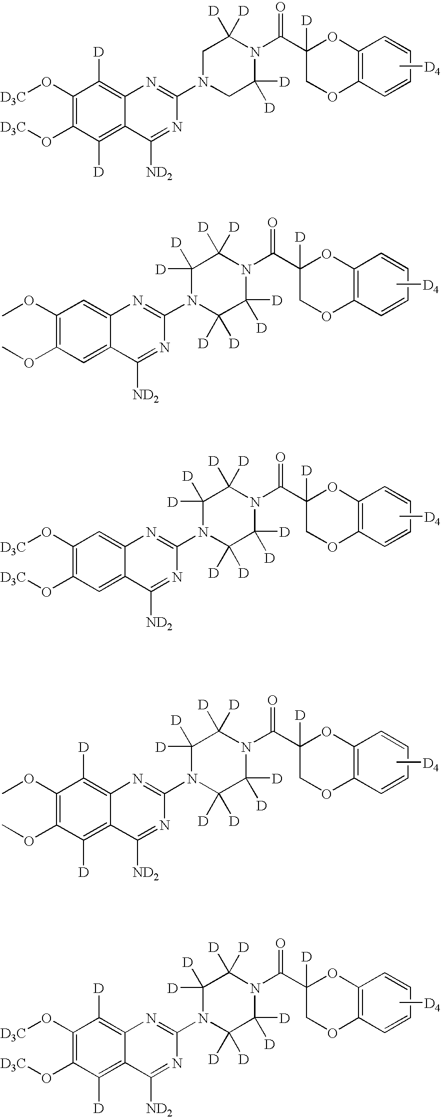

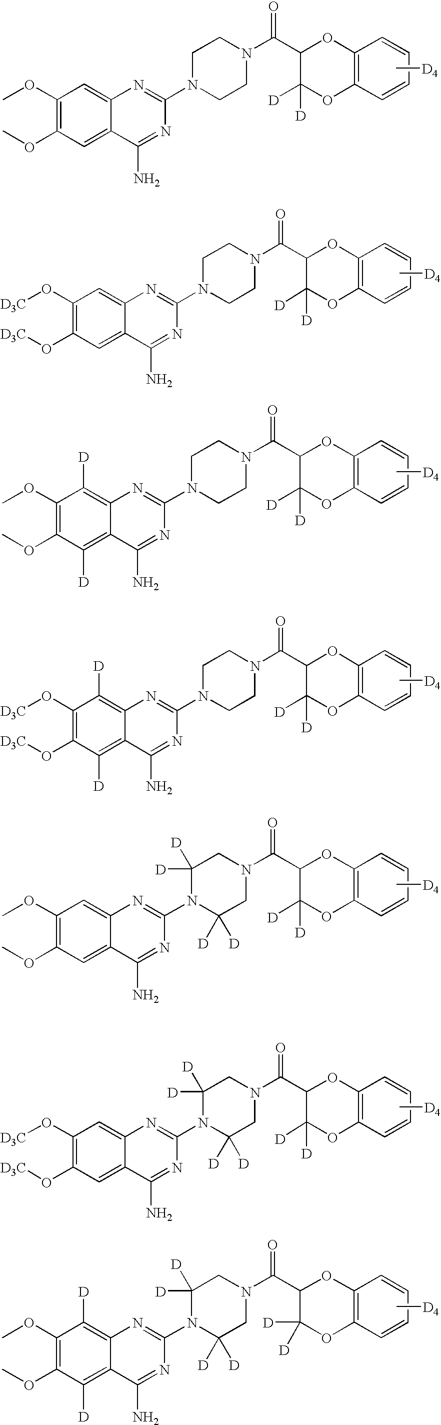

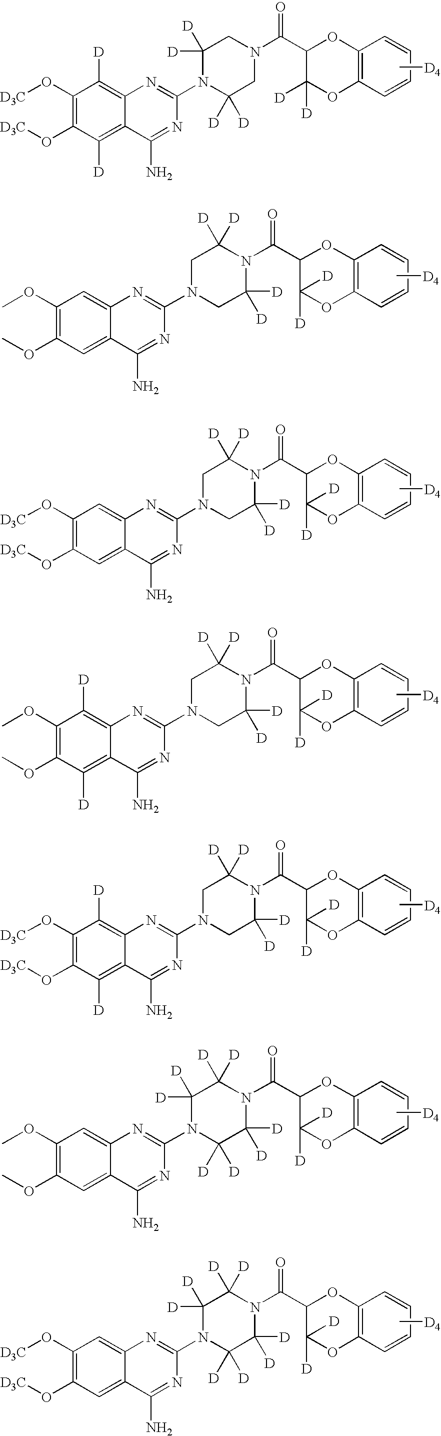

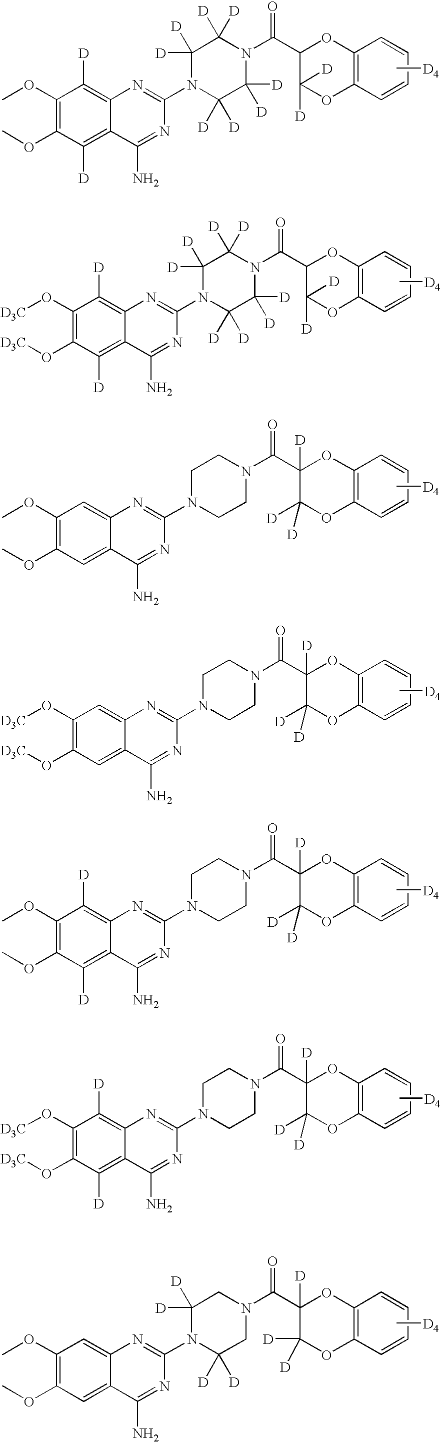

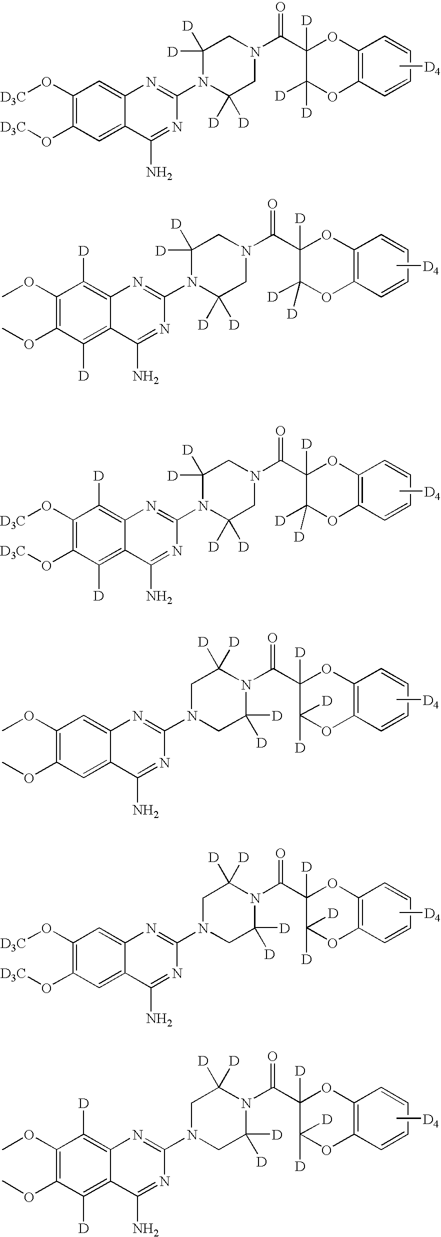

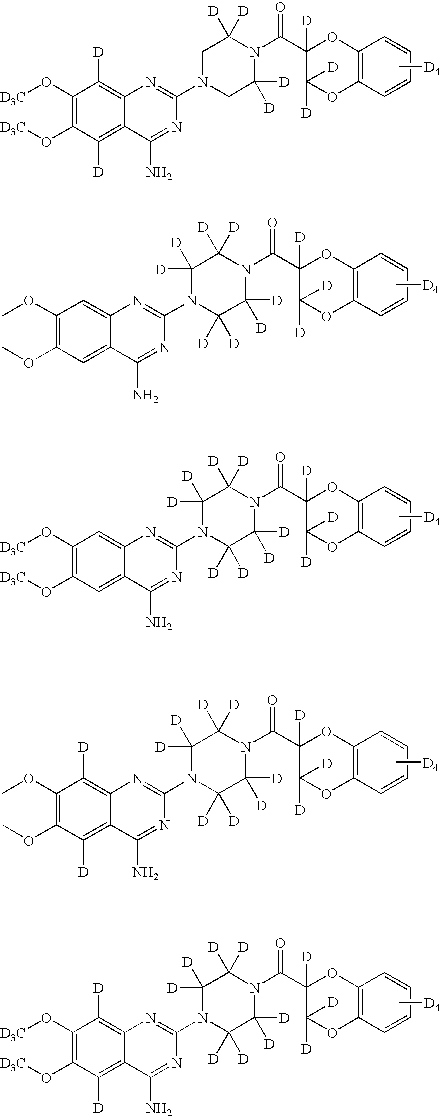

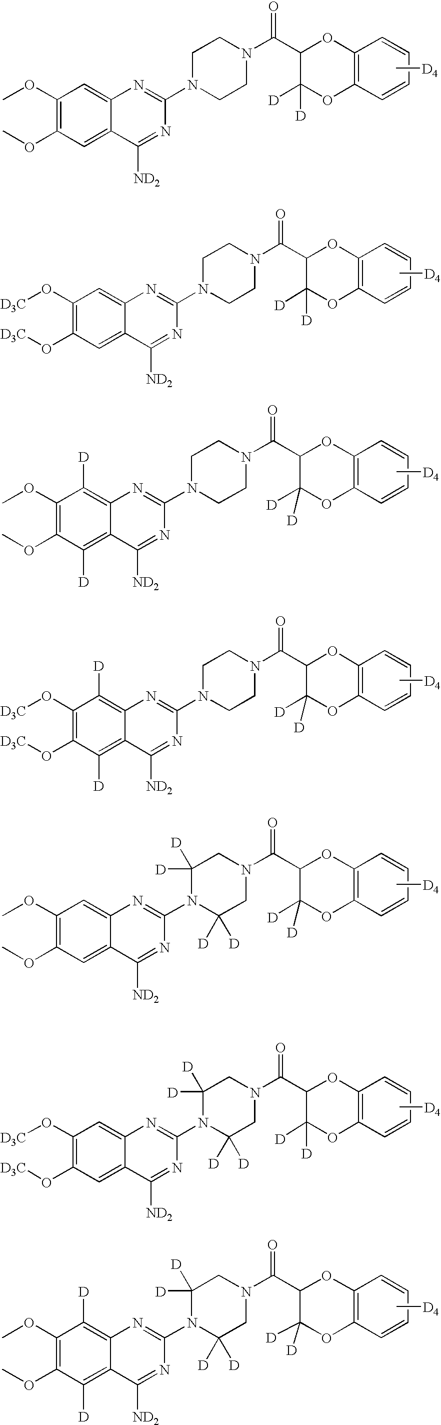

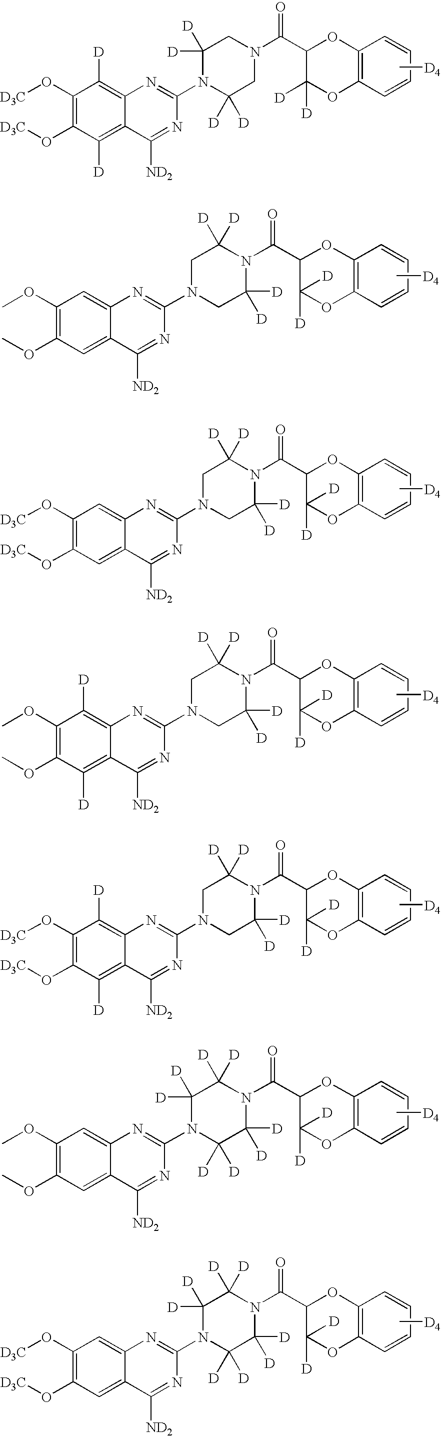

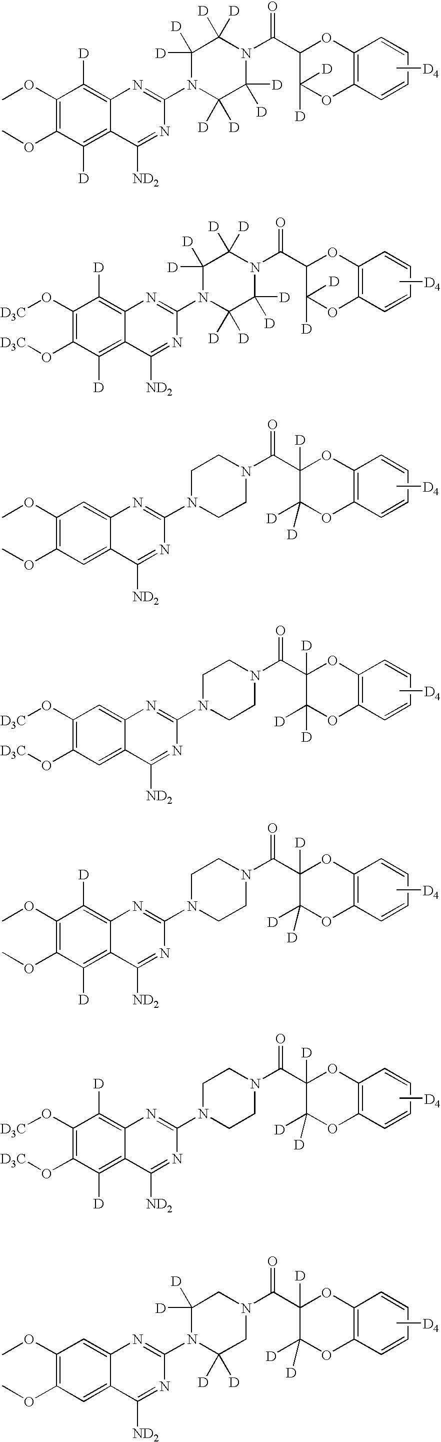

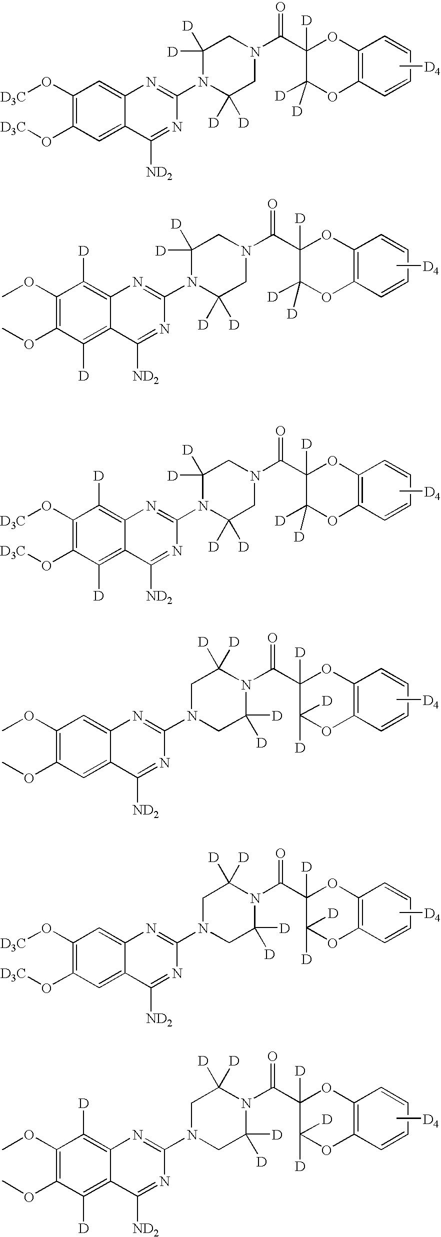

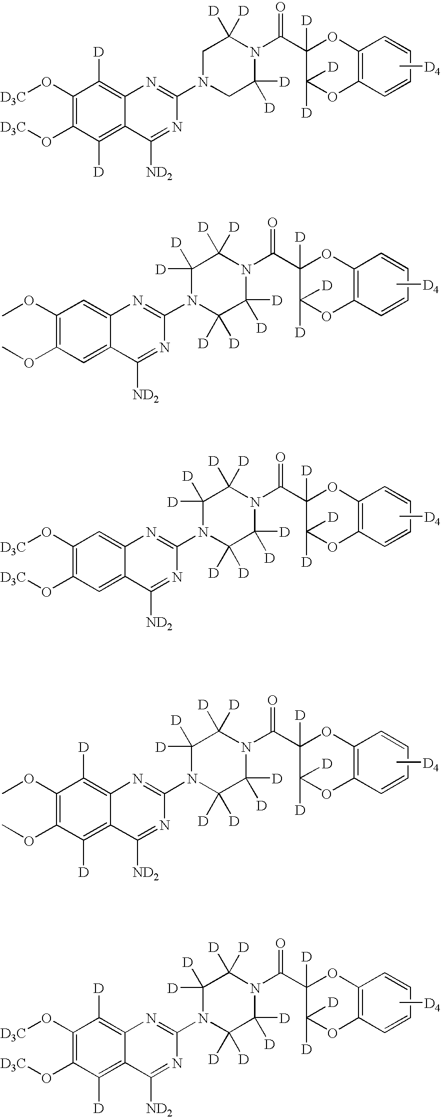

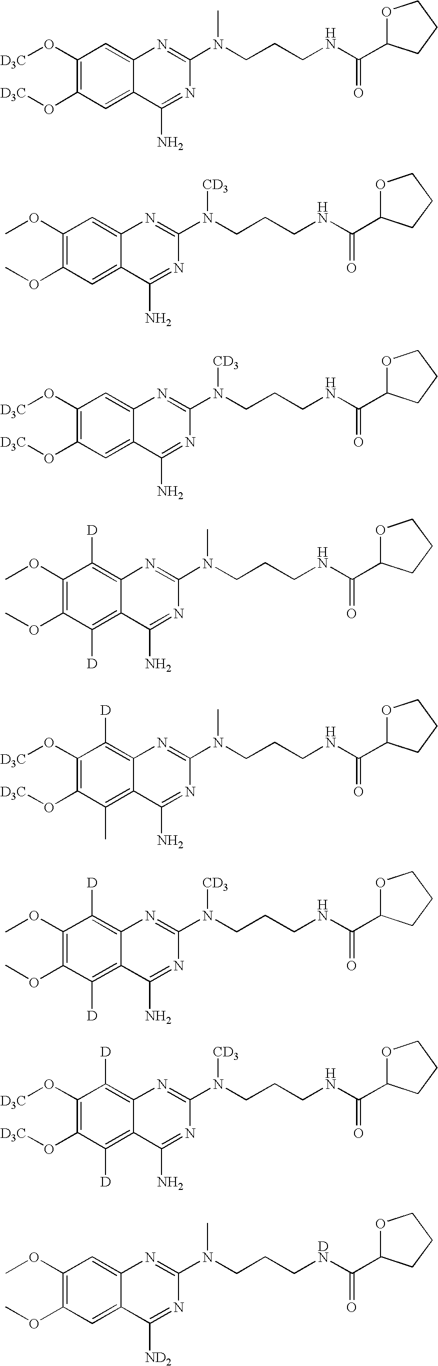

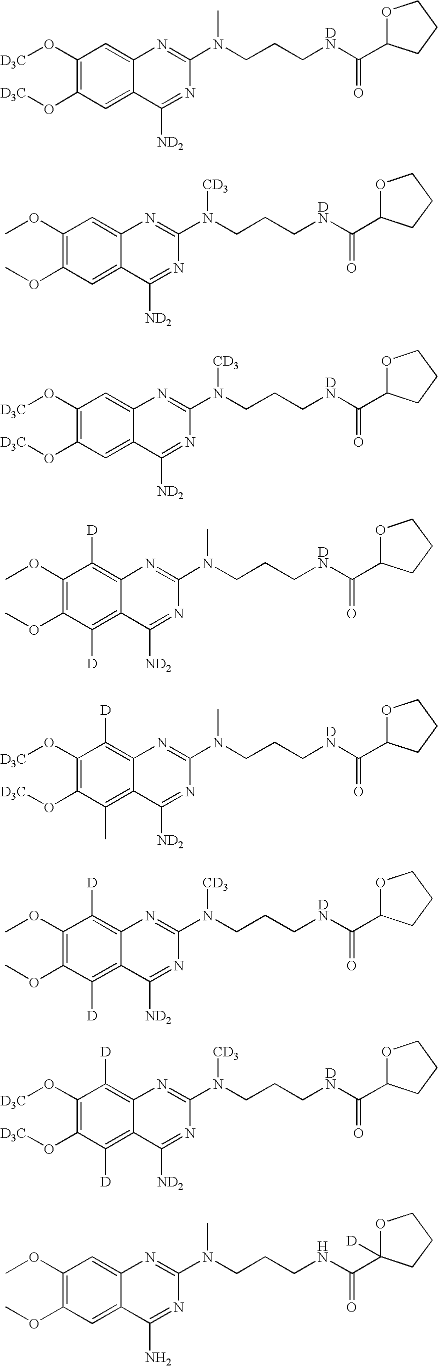

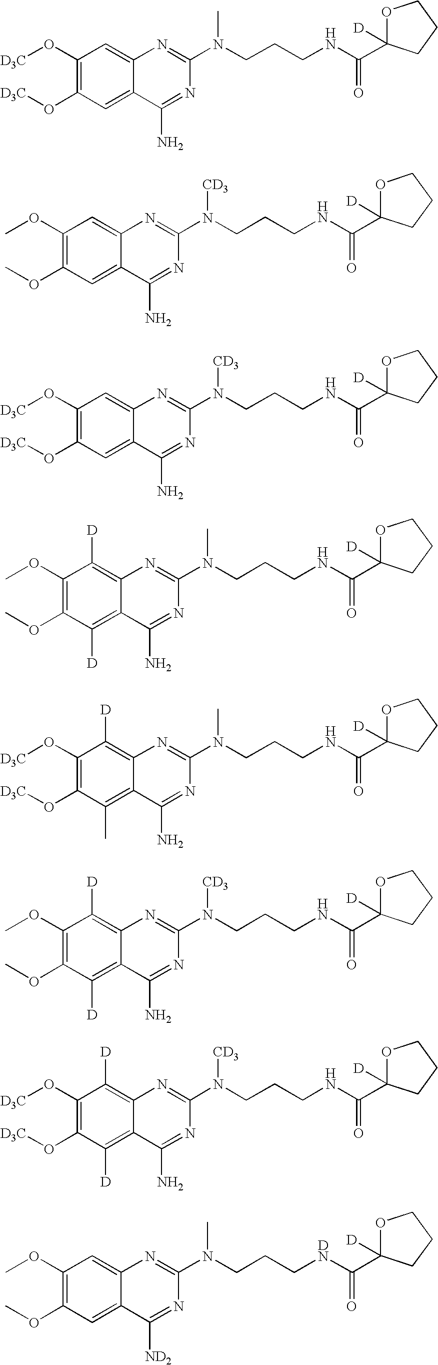

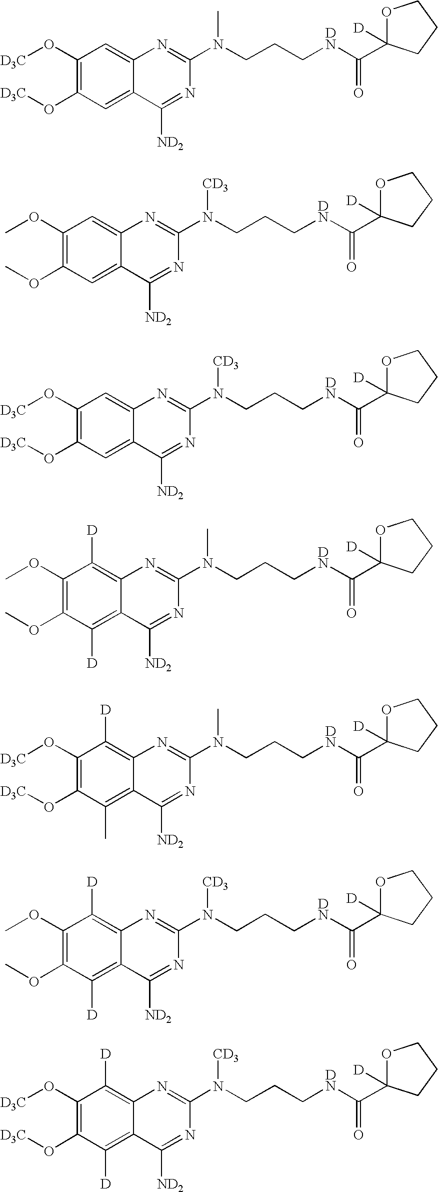

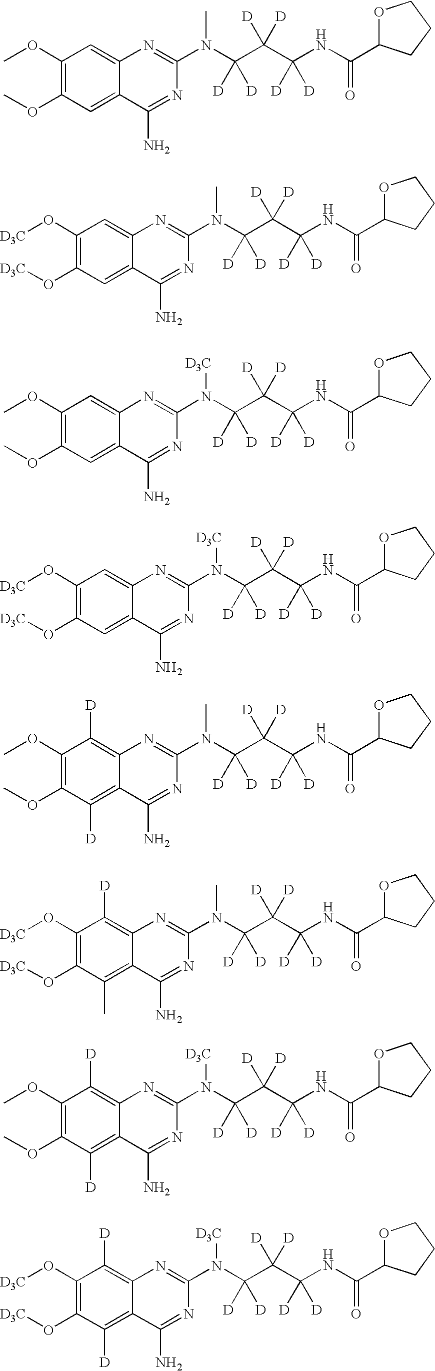

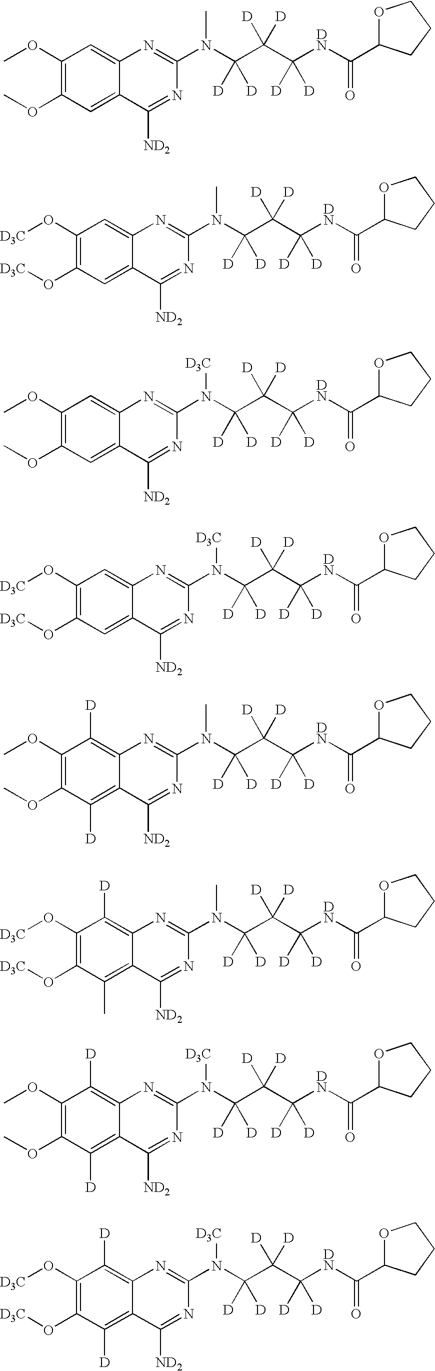

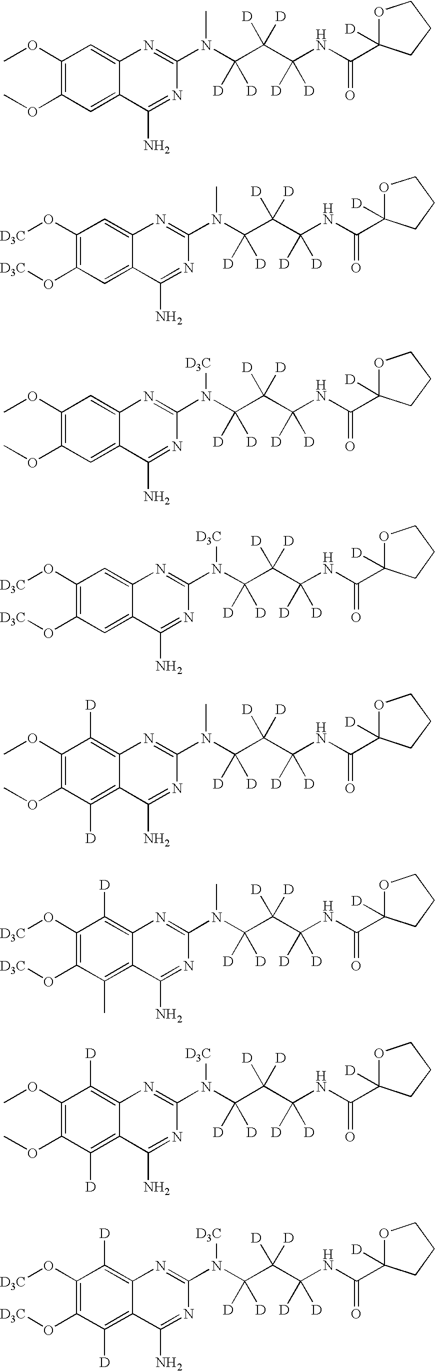

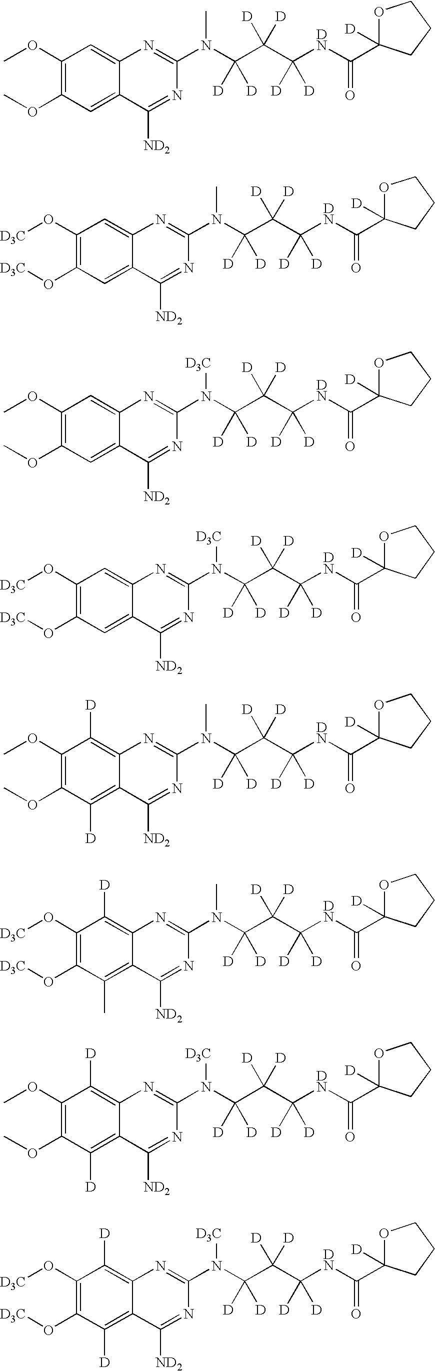

or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers, or a pharmaceutically acceptable salt, solvate, or prodrug thereof.

In another embodiment, are provided compounds according to Formula 1 having one of the following structures:

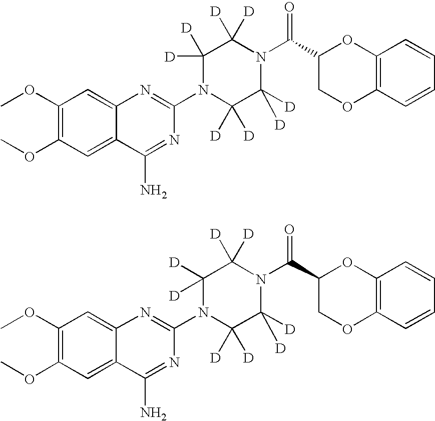

or a pharmaceutically acceptable salt, solvate, or prodrug thereof.

In certain embodiments, R₁ is hydrogen. In other embodiments, R₄ is hydrogen. In some embodiments, R₅ is hydrogen. In other embodiments, R₆ is hydrogen. In yet other embodiments, R₈ is hydrogen. In still other embodiments, R₉ is hydrogen. In some embodiments, R₁₀ is hydrogen. In other embodiments, R₁₁ is hydrogen. In yet other embodiments, R₁₂ is hydrogen. In still other embodiments, R₁₃ is hydrogen. In yet other embodiments, R₁₄ is hydrogen. In yet other embodiments, R₁₅ is hydrogen. In still other embodiments, R₁₆ is hydrogen. In other embodiments, R₁₇ is hydrogen. In yet other embodiments, R₁₈ is hydrogen. In still other embodiments, R₁₉ is hydrogen. In yet other embodiments, R₂₀ is hydrogen. In other embodiments, R₂₁ is hydrogen. In yet other embodiments, R₂₂ is hydrogen. In still other embodiments, R₂₃ is hydrogen. In yet other embodiments, R₂₄ is hydrogen. In yet other embodiments, R₂₅ is hydrogen. In still other embodiments, R₂₆ is hydrogen. In other embodiments, R₂₇ is hydrogen. In yet other embodiments, R₂₈ is hydrogen. In still other embodiments, R₂₉ is hydrogen. In yet other embodiments, R₃₀ is hydrogen. In yet other embodiments, R₃₁ is hydrogen. In yet other embodiments, R₃₂ is hydrogen. In still other embodiments, R₃₃ is hydrogen. In some embodiments, R₃₄ is hydrogen. In other embodiments, R₃₅ is hydrogen. In yet other embodiments, R₃₆ is hydrogen. In still other embodiments, R₃₇ is hydrogen. In yet other embodiments, R₃₈ is hydrogen. In yet other embodiments, R₃₉ is hydrogen. In still other embodiments, R₄₀ is hydrogen. In other embodiments, R₄₁ is hydrogen. In yet other embodiments, R₄₂ is hydrogen. In still other embodiments, R₄₃ is hydrogen. In yet other embodiments, R₄₄ is hydrogen. In other embodiments, R₄₅ is hydrogen. In yet other embodiments, R₄₆ is hydrogen. In still other embodiments, R₄₇ is hydrogen. In yet other embodiments, R₄₈ is hydrogen. In yet other embodiments, R₄₉ is hydrogen. In still other embodiments, R₅₀ is hydrogen. In other embodiments, R₅₁ is hydrogen. In yet other embodiments, R₅₂ is hydrogen. In still other embodiments, R₅₃ is hydrogen. In yet other embodiments, R₅₄ is hydrogen. In yet other embodiments, R₅₅ is hydrogen. In yet other embodiments, R₅₆ is hydrogen. In still other embodiments, R₅₇ is hydrogen. In some embodiments, R₅₈ is hydrogen. In other embodiments, R₅₉ is hydrogen. In yet other embodiments, R₆₀ is hydrogen. In still other embodiments, R₆₁ is hydrogen. In yet other embodiments, R₆₂ is hydrogen. In yet other embodiments, R₆₃ is hydrogen. In still other embodiments, R₆₄ is hydrogen.

In certain embodiments, R₁ is deuterium. In other embodiments, R₄is deuterium. In some embodiments, R₅ is deuterium. In other embodiments, R₆ is deuterium. In yet other embodiments, R₈ is deuterium. In still other embodiments, R₉ is deuterium. In some embodiments, R₁₀ is deuterium. In other embodiments, R₁₁ is deuterium. In yet other embodiments, R₁₂ is deuterium. In still other embodiments, R₁₃ is deuterium. In yet other embodiments, R₁₄ is deuterium. In yet other embodiments, R₁₅ is deuterium. In still other embodiments, R₁₆ is deuterium. In other embodiments, R₁₇ is deuterium. In yet other embodiments, R₁₈ is deuterium. In still other embodiments, R₁₉ is deuterium. In yet other embodiments, R₂₀ is deuterium. In other embodiments, R₂₁ is deuterium. In yet other embodiments, R₂₂ is deuterium. In still other embodiments, R₂₃ is deuterium. In yet other embodiments, R₂₄ is deuterium. In yet other embodiments, R₂₅ is deuterium. In still other embodiments, R₂₆ is deuterium. In other embodiments, R₂₇ is deuterium. In yet other embodiments, R₂₈ is deuterium. In still other embodiments, R₂₉ is deuterium. In yet other embodiments, R₃₀ is deuterium. In yet other embodiments, R₃₁ is deuterium. In yet other embodiments, R₃₂ is deuterium. In still other embodiments, R₃₃ is deuterium. In some embodiments, R₃₄ is deuterium. In other embodiments, R₃₅ is deuterium. In yet other embodiments, R₃₆ is deuterium. In still other embodiments, R₃₇ is deuterium. In yet other embodiments, R₃₈ is deuterium. In yet other embodiments, R₃₉ is deuterium. In still other embodiments, R₄₀ is deuterium. In other embodiments, R₄₁ is deuterium. In yet other embodiments, R₄₂ is deuterium. In still other embodiments, R₄₃ is deuterium. In yet other embodiments, R₄₄ is deuterium. In other embodiments, R₄₅ is deuterium. In yet other embodiments, R₄₆ is deuterium. In still other embodiments, R₄₇ is deuterium. In yet other embodiments, R₄₈ is deuterium. In yet other embodiments, R₄₉ is deuterium. In still other embodiments, R₅₀ is deuterium. In other embodiments, R₅₁ is deuterium. In yet other embodiments, R₅₂ is deuterium. In still other embodiments, R₅₃ is deuterium. In yet other embodiments, R₅₄ is deuterium. In yet other embodiments, R₅₅ is deuterium. In yet other embodiments, R₅₆ is deuterium. In still other embodiments, R₅₇ is deuterium. In some embodiments, R₅₈ is deuterium. In other embodiments, R₅₉ is deuterium. In yet other embodiments, R₆₀ is deuterium. In still other embodiments, R₆₁ is deuterium. In yet other embodiments, R₆₂ is deuterium. In yet other embodiments, R₆₃ is deuterium. In still other embodiments, R₆₄ is deuterium.

In certain embodiments, R₂ is —CH₃. In other embodiments, R₃ is —CH₃.

In certain embodiments, R₂ is —CH₂D. In other embodiments, R₃ is —CH₂D.

In certain embodiments, R₂ is —CHD₂. In other embodiments, R₃ is —CHD₂.

In certain embodiments, R₂ is —CD₃. In other embodiments, R₃ is —CD₃.

In certain embodiments, R₇ is

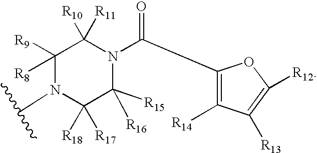

In certain embodiments, R₇ is

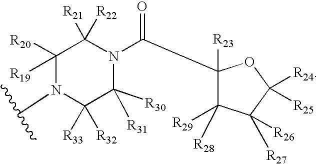

In certain embodiments, R₇ is

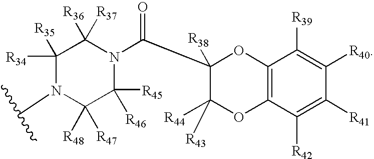

In certain embodiments, R₇ is

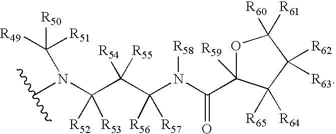

In certain embodiments, R₁ is not hydrogen. In other embodiments, R₄ is not hydrogen. In some embodiments, R₅ is not hydrogen. In other embodiments, R₆ is not hydrogen. In yet other embodiments, R₈ is not hydrogen. In still other embodiments, R₉ is not hydrogen. In some embodiments, R₁₀ is not hydrogen. In other embodiments, R₁₁ is not hydrogen. In yet other embodiments, R₁₂ is not hydrogen. In still other embodiments, R₁₃ is not hydrogen. In yet other embodiments, R₁₄ is not hydrogen. In yet other embodiments, R₁₅ is not hydrogen. In still other embodiments, R₁₆ is not hydrogen. In other embodiments, R₁₇ is not hydrogen. In yet other embodiments, R₁₈ is not hydrogen. In still other embodiments, R₁₉ is not hydrogen. In yet other embodiments, R₂₀ is not hydrogen. In other embodiments, R₂₁ is not hydrogen. In yet other embodiments, R₂₂ is not hydrogen. In still other embodiments, R₂₃ is not hydrogen. In yet other embodiments, R₂₄ is not hydrogen. In yet other embodiments, R₂₅ is not hydrogen. In still other embodiments, R₂₆ is not hydrogen. In other embodiments, R₂₇ is not hydrogen. In yet other embodiments, R₂₈ is not hydrogen. In still other embodiments, R₂₉ is not hydrogen. In yet other embodiments, R₃₀ is not hydrogen. In yet other embodiments, R₃₁ is not hydrogen. In yet other embodiments, R₃₂ is not hydrogen. In still other embodiments, R₃₃ is not hydrogen. In some embodiments, R₃₄ is not hydrogen. In other embodiments, R₃₅ is not hydrogen. In yet other embodiments, R₃₆ is not hydrogen. In still other embodiments, R₃₇ is not hydrogen. In yet other embodiments, R₃₈ is not hydrogen. In yet other embodiments, R₃₉ is not hydrogen. In still other embodiments, R₄₀ is not hydrogen. In other embodiments, R₄₁ is not hydrogen. In yet other embodiments, R₄₂ is not hydrogen. In still other embodiments, R₄₃ is not hydrogen. In yet other embodiments, R₄₄ is not hydrogen. In other embodiments, R₄₅ is not hydrogen. In yet other embodiments, R₄₆ is not hydrogen. In still other embodiments, R₄₇ is not hydrogen. In yet other embodiments, R₄₈ is not hydrogen. In yet other embodiments, R₄₉ is not hydrogen. In still other embodiments, R₅₀ is not hydrogen. In other embodiments, R₅₁ is not hydrogen. In yet other embodiments, R₅₂ is not hydrogen. In still other embodiments, R₅₃ is not hydrogen. In yet other embodiments, R₅₄ is not hydrogen. In yet other embodiments, R₅₅ is not hydrogen. In yet other embodiments, R₅₆ is not hydrogen. In still other embodiments, R₅₇ is not hydrogen. In some embodiments, R₅₈ is not hydrogen. In other embodiments, R₅₉ is not hydrogen. In yet other embodiments, R₆₀ is not hydrogen. In still other embodiments, R₆₁ is not hydrogen. In yet other embodiments, R₆₂ is not hydrogen. In yet other embodiments, R₆₃ is not hydrogen. In still other embodiments, R₆₄ is not hydrogen.

In certain embodiments, R₁ is not deuterium. In other embodiments, R₄ is not deuterium. In some embodiments, R₅ is not deuterium. In other embodiments, R₆ is not deuterium. In yet other embodiments, R₈ is not deuterium. In still other embodiments, R₉ is not deuterium. In some embodiments, R₁₀ is not deuterium. In other embodiments, R₁₁ is not deuterium. In yet other embodiments, R₁₂ is not deuterium. In still other embodiments, R₁₃ is not deuterium. In yet other embodiments, R₁₄ is not deuterium. In yet other embodiments, R₁₅ is not deuterium. In still other embodiments, R₁₆ is not deuterium. In other embodiments, R₁₇ is not deuterium. In yet other embodiments, R₁₈ is not deuterium. In still other embodiments, R₁₉ is not deuterium. In yet other embodiments, R₂₀ is not deuterium. In other embodiments, R₂₁ is not deuterium. In yet other embodiments, R₂₂ is not deuterium. In still other embodiments, R₂₃ is not deuterium. In yet other embodiments, R₂₄ is not deuterium. In yet other embodiments, R₂₅ is not deuterium. In still other embodiments, R₂₆ is not deuterium. In other embodiments, R₂₇ is not deuterium. In yet other embodiments, R₂₈ is not deuterium. In still other embodiments, R₂₉ is not deuterium. In yet other embodiments, R₃₀ is not deuterium. In yet other embodiments, R₃₁ is not deuterium. In yet other embodiments, R₃₂ is not deuterium. In still other embodiments, R₃₃ is not deuterium. In some embodiments, R₃₄ is not deuterium. In other embodiments, R₃₅ is not deuterium. In yet other embodiments, R₃₆ is not deuterium. In still other embodiments, R₃₇ is not deuterium. In yet other embodiments, R₃₈ is not deuterium. In yet other embodiments, R₃₉ is not deuterium. In still other embodiments, R₄₀ is not deuterium. In other embodiments, R₄₁ is not deuterium. In yet other embodiments, R₄₂ is not deuterium. In still other embodiments, R₄₃ is not deuterium. In yet other embodiments, R₄₄ is not deuterium. In other embodiments, R₄₅ is not deuterium. In yet other embodiments, R₄₆ is not deuterium. In still other embodiments, R₄₇ is not deuterium. In yet other embodiments, R₄₈ is not deuterium. In yet other embodiments, R₄₉ is not deuterium. In still other embodiments, R₅₀ is not deuterium. In other embodiments, R₅₁ is not deuterium. In yet other embodiments, R₅₂ is not deuterium. In still other embodiments, R₅₃ is not deuterium. In yet other embodiments, R₅₄ is not deuterium. In yet other embodiments, R₅₅ is not deuterium. In yet other embodiments, R₅₆ is not deuterium. In still other embodiments, R₅₇ is not deuterium. In some embodiments, R₅₈ is not deuterium. In other embodiments, R₅₉ is not deuterium. In yet other embodiments, R₆₀ is not deuterium. In still other embodiments, R₆₁ is not deuterium. In yet other embodiments, R₆₂ is not deuterium. In yet other embodiments, R₆₃ is not deuterium. In still other embodiments, R₆₄ is not deuterium.

In certain embodiments, R₂ is not —CH₃. In other embodiments, R₃ is not —CH₃.

In certain embodiments, R₂ is not —CH₂D. In other embodiments, R₃ is not —CH₂D.

In certain embodiments, R₂ is not —CHD₂. In other embodiments, R₃ is not —CHD₂.

In certain embodiments, R₂ is not —CD₃. In other embodiments, R₃ is not —CD₃.

In certain embodiments, R₇ is not

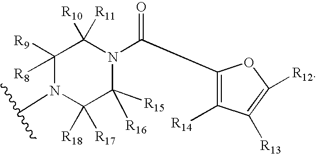

In certain embodiments, R₇ is not

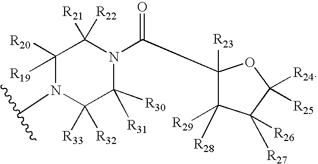

In certain embodiments, R₇ is not

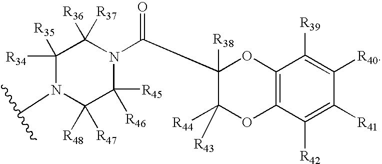

In certain embodiments, R₇ is not

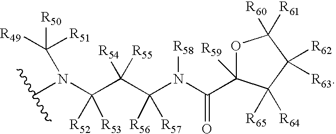

In some embodiments, are pharmaceutical compositions comprising a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof, and one or more pharmaceutically acceptable excipients or carriers.

In other embodiments, are provided pharmaceutical compositions comprising a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof, and one or more pharmaceutically acceptable excipients or carriers for enteral, intravenous infusion, parenteral, topical or ocular administration.

In yet other embodiments, are provided pharmaceutical compositions comprising a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof, and one or more pharmaceutically acceptable excipients or carriers for the treatment of conditions in which it is beneficial to modulate an alpha-adrenergic receptor.

In yet another embodiment, there are provided pharmaceutical compositions comprising a compound of Formula 1, or a single enantiomer according to Formula 1, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer according to Formula 1, or a mixture of diastereomers, or a pharmaceutically acceptable salt, solvate, or prodrug thereof, in a pharmaceutically acceptable vehicle, carrier, diluent, or excipient, or a combination thereof, for the treatment of conditions in which it is beneficial to modulate an alpha-adrenergic receptor.

In another embodiment, there are provided methods of modulating alpha-adrenergic receptors, with one or more of the compounds or compositions of Formula 1, or a single enantiomer according to Formula 1, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer according to Formula 1, or a mixture of diastereomers, or a pharmaceutically acceptable salt, solvate, or prodrug thereof.

In certain embodiments, the compound of Formula 1 contains about 60% or more by weight of the (−)-enantiomer of the compound and about 40% or less by weight of (+)-enantiomer of the compound. In certain embodiments, the compound of Formula 1 contains about 70% or more by weight of the (−)-enantiomer of the compound and about 30% or less by weight of (+)-enantiomer of the compound. In certain embodiments, the compound of Formula 1 contains about 80% or more by weight of the (−)-enantiomer of the compound and about 20% or less by weight of (+)-enantiomer of the compound. In certain embodiments, the compound of Formula 1 contains about 90% or more by weight of the (−)-enantiomer of the compound and about 10% or less by weight of the (+)-enantiomer of the compound. In certain embodiments, the compound of Formula 1 contains about 95% or more by weight of the (−)-enantiomer of the compound and about 5% or less by weight of (+)-enantiomer of the compound. In certain embodiments, the compound of Formula 1 contains about 99% or more by weight of the (−)-enantiomer of the compound and about 1% or less by weight of (+)-enantiomer of the compound.

In certain embodiments, the compound of Formula 1 contains about 60% or more by weight of the (+)-enantiomer of the compound and about 40% or less by weight of (−)-enantiomer of the compound. In certain embodiments, the compound of Formula 1 contains about 70% or more by weight of the (+)-enantiomer of the compound and about 30% or less by weight of (−)-enantiomer of the compound. In certain embodiments, the compound of Formula 1 contains about 80% or more by weight of the (+)-enantiomer of the compound and about 20% or less by weight of (−)-enantiomer of the compound. In certain embodiments, the compound of Formula 1 contains about 90% or more by weight of the (+)-enantiomer of the compound and about 10% or less by weight of the (−)-enantiomer of the compound. In certain embodiments, the compound of Formula 1 contains about 95% or more by weight of the (+)-enantiomer of the compound and about 5% or less by weight of (−)-enantiomer of the compound. In certain embodiments, the compound of Formula 1 contains about 99% or more by weight of the (+)-enantiomer of the compound and about 1% or less by weight of (−)-enantiomer of the compound.

The deuterated compound of Formula 1 may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, without being bound by any theory, the compound provided herein may expose a patient to a maximum of about 0.000005% D₂O or about 0.00001% DHO, assuming that all of the C-D bonds in the compound of Formula 1 are metabolized and released as D₂O or DHO. This quantity is a small fraction of the naturally occurring background levels of D₂O or DHO in circulation. In certain embodiments, the levels of D₂O shown to cause toxicity in animals is much greater than even the maximum limit of exposure because of the deuterium enriched compound of Formula 1. Thus, in certain embodiments, the deuterium-enriched compound provided herein should not cause any additional toxicity because of the use of deuterium.

In one embodiment, the deuterated compounds provided herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

Isotopic hydrogen can be introduced into a compound of Formula 1 as provided herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. In addition, the molecule being labeled may be changed, depending upon the severity of the synthetic reaction employed. Exchange techniques, on the other hand, may yield lower tritium or deuterium incorporation, often with the isotope being distributed over many sites on the molecule, but offer the advantage that they do not require separate synthetic steps and are less likely to disrupt the structure of the molecule being labeled.

The compounds of Formula 1 as provided herein can be prepared by methods known to one of skill in the art or following procedures similar to those described in the Example section herein and routine modifications thereof. For an example, a compound of Formula 1 can be prepared as shown in Scheme 2.

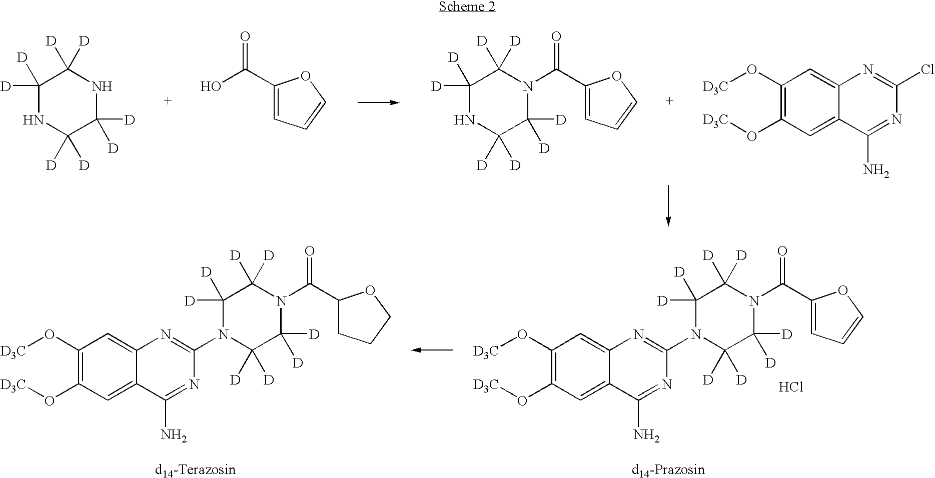

Deuterium can be incorporated to different positions synthetically, according to the synthetic procedures as shown in Scheme 2, by using appropriate deuterated intermediates. For example, to introduce deuterium at various substituted positions, intermediates with the corresponding deuterium substitutions can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as the carboxyl, via proton-deuterium equilibrium exchange.

Certain molecular structures described herein may occur as abbreviations. One such example is the molecular structure

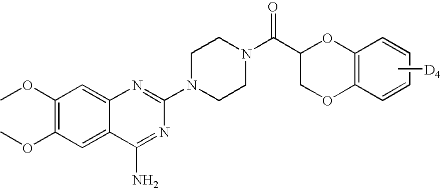

which signifies the presence of four deuterium atoms on the phenyl ring and is equivalent to the molecular structure

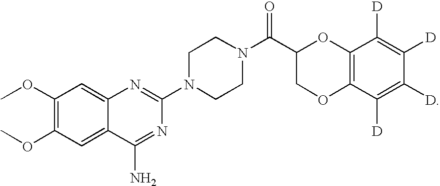

Exemplary conditions for forming and removing suitable nitrogen protecting groups may be found in Greene and Wuts, Protective Groups in Organic Synthesis, 3^{rd} Ed., John Wiley & Sons, New York, N.Y., 1999. Suitable nitrogen protecting groups include but are not limited to those selected from methoxymethyl (MOM), benzyloxymethyl (BOM), 2-(trimethylsilyl)ethoxymethyl (SEM), methoxyethoxymethyl (MEM), or t-butyl groups. In addition, exemplary conditions for forming and removing suitable carboxylic acid protecting groups may be found in Greene and Wuts, Protective Groups in Organic Synthesis, 3^{rd} Ed., John Wiley & Sons, New York, N.Y., 1999.

It is to be understood that the compounds provided herein may contain one or more chiral centers, chiral axes, and/or chiral planes, as described in “Stereochemistry of Carbon Compounds” Eliel and Wilen, John Wiley & Sons, New York, 1994, pp. 1119-1190. Such chiral centers, chiral axes, and chiral planes may be of either the (R) or (S) configuration, or may be a mixture thereof.

Another method for characterizing a composition containing a compound having at least one chiral center is by the effect of the composition on a beam of polarized light. When a beam of plane polarized light is passed through a solution of a chiral compound, the plane of polarization of the light that emerges is rotated relative to the original plane. This phenomenon is known as optical activity, and compounds that rotate the plane of polarized light are said to be optically active. One enantiomer of a compound will rotate the beam of polarized light in one direction, and the other enantiomer will rotate the beam of light in the opposite direction. The enantiomer that rotates the polarized light in the clockwise direction is the (+)-enantiomer, and the enantiomer that rotates the polarized light in the counterclockwise direction is the (−)-enantiomer. Included within the scope of the compositions described herein are compositions containing between 0 and 100% of the (+) and/or (−)-enantiomer of compounds of Formula 1.

Where a compound of Formula 1 contains an alkenyl or alkenylene group, the compound may exist as one or mixture of geometric cis/trans (or Z/E) isomers. Where structural isomers are interconvertible via a low energy barrier, the compound of Formula 1 may exist as a single tautomer or a mixture of tautomers. This can take the form of proton tautomerism in the compound of Formula 1 that contains for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the compound of Formula 1 contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compound of Formula 1 may also be provided as a prodrug, which is a functional derivative of the compound of Formula 1 and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al.,Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev.1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

### Pharmaceutical Compositions

Provided herein are pharmaceutical compositions comprising a compound of Formula 1 as an active ingredient, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers.

Also provided herein are pharmaceutical compositions comprising a compound of Formula 1 as an active ingredient, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers, for the treatment of a condition involving the modulation of an alpha-adrenergic receptor.

Provided herein are pharmaceutical compositions in modified release dosage forms, which comprise a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions may also comprise non-release controlling excipients.

Further provided herein are pharmaceutical compositions in enteric coated dosage forms, which comprise a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; and one or more release controlling excipients for use in an enteric coated dosage form. The pharmaceutical compositions may also comprise non-release controlling excipients.

Further provided herein are pharmaceutical compositions in effervescent dosage forms, which comprise a compounds of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; and one or more release controlling excipients for use in an enteric coated dosage form. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; and one or more release controlling and non-release controlling excipients, such as those excipients suitable for a disruptable semi-permeable membrane and as swellable substances.

Provided herein also are pharmaceutical compositions in a dosage form for oral administration to a subject, which comprises a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of one or more compounds of Formula 1 in the form of enteric-coated granules, as delayed-release capsules for oral administration. The pharmaceutical compositions further comprise cellulose, disodium hydrogen phosphate, hydroxypropyl cellulose, hypromellose, lactose, mannitol, and sodium lauryl sulfate.

Provided herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of one or more compounds of Formula 1 in the form of enteric-coated pellets, as delayed-release capsules for oral administration. The pharmaceutical compositions further comprise glyceryl monostearate 40-50, hydroxypropyl cellulose, hypromellose, magnesium stearate, methacrylic acid copolymer type C, polysorbate 80, sugar spheres, talc, and triethyl citrate.

Provided herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of one or more compounds of Formula 1, as enteric-coated delayed-release tablets for oral administration. The pharmaceutical compositions further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

Provided herein are pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of one or more compounds of Formula 1, as enteric-coated delayed-release tablets for oral administration. The pharmaceutical compositions further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compound of Formula 1 provided herein may be administered alone, or in combination with one or more other compounds provided herein, one or more other active ingredients. The pharmaceutical compositions that comprise a compound provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

### A. Oral Administration

The pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Micellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as other alpha-adrenergic receptor modulators.

### B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

The pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

The pharmaceutical compositions provided herein for topical administration may be formulated to be immediate release or modified release, including delayed-, sustained-, pulsed-, controlled-, targeted, and programmed release.

### D. Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol,; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, β glucan acetate, β glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients.

### 3. Multiparticulate Controlled Release Devices

The pharmaceutical compositions provided herein in a modified release dosage form may be fabricated a multiparticulate controlled release device, which comprises a multiplicity of particles, granules, or pellets, ranging from about 10 μm to about 3 μm, about 50 μm to about 2.5 mm, or from about 100 μm to about 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

Provided are methods for treating, preventing, or ameliorating one or more symptoms of hypertension, cardiac failure, prostatitis, and/or benign prostatic hyperplasia comprising administering to a subject having or being suspected to have such a disease, a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof.

Symptoms of cardiac failure, include, but are not limited to, dyspnea, orthopnea, fatigue, nocturnal cough, confusion and memory impairment. Symptoms of prostatitis, include, but are not limited to, chills, fever, body aches, pain in the lower back and genital area, and burning or painful urination. Symptoms of prostatic hyperplasia, include, but are not limited to, nocturia, urgency, hesitancy, intermittency, incomplete voiding, weak urinary system, and straining.

In one embodiment is a method for the treatment, prevention, or amelioration of one or more symptoms of an alpha-adrenergic receptor-mediated disease, the method comprising administering a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof.

Provided herein are methods for treating a subject, including a human, having or suspected of having a disease involving hypertension, cardiac failure, prostatitis, and/or benign prostatic hyperplasia, or for preventing such disease in a subject prone to the disease; comprising administering to the subject a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; so as to affect decreased inter-individual variation in plasma levels of the compound or a metabolite thereof, during the treatment of the disease as compared to the corresponding non-isotopically enriched compound.

In certain embodiments, the inter-individual variation in plasma levels of the compounds of Formula 1, or metabolites thereof, is decreased by greater than about 5%, greater than about 10%, greater than about 20%, greater than about 30%, greater than about 40%, or by greater than about 50% as compared to the corresponding non-isotopically enriched compound.

Provided herein are methods for treating a subject, including a human, having or suspected of having a disease involving hypertension, cardiac failure, prostatitis, and/or benign prostatic hyperplasia, or for preventing such disease in a subject prone to the disease; comprising administering to the subject a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; so as to affect increased average plasma levels of the compound or decreased average plasma levels of at least one metabolite of the compound per dosage unit as compared to the corresponding non-isotopically enriched compound.

In certain embodiments, the average plasma levels of the compound of Formula 1 are increased by greater than about 5%, greater than about 10%, greater than about 20%, greater than about 30%, greater than about 40%, or greater than about 50% as compared to the corresponding non-isotopically enriched compounds.

In certain embodiments, the average plasma levels of a metabolite of the compound of Formula 1 are decreased by greater than about 5%, greater than about 10%, greater than about 20%, greater than about 30%, greater than about 40%, or greater than about 50% as compared to the corresponding non-isotopically enriched compounds.

Plasma levels of the compound of Formula 1, or metabolites thereof, are measured using the methods described by Li et al. (Rapid Communications in Mass Spectrometry 2005, 19, 1943-1950).

Provided herein are methods for treating a subject, including a human, having or suspected of having a disease involving hypertension, cardiac failure, prostatitis, and/or benign prostatic hyperplasia, or for preventing such disease in a subject prone to the disease; comprising administering to the subject a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; so as to affect a decreased inhibition of, and/or metabolism by at least one cytochrome P₄₅₀ isoform in the subject during the treatment of the disease as compared to the corresponding non-isotopically enriched compound.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In certain embodiments, the decrease in inhibition of the cytochrome P450 isoform by a compound of Formula 1 is greater than about 5%, greater than about 10%, greater than about 20%, greater than about 30%, greater than about 40%, or greater than about 50% as compared to the corresponding non-isotopically enriched compounds.

The inhibition of the cytochrome P₄₅₀ isoform is measured by the method of Ko et al. (British Journal of Clinical Pharmacology, 2000, 49, 343-351).

Provided herein are methods for treating a subject, including a human, having or suspected of having a disease involving hypertension, cardiac failure, prostatitis, and/or benign prostatic hyperplasia, or for preventing such disease in a subject prone to the disease; comprising administering to the subject a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; so as to affect a decreased metabolism via at least one polymorphically-expressed cytochrome P₄₅₀ isoform in the subject during the treatment of the disease as compared to the corresponding non-isotopically enriched compound.

Examples of polymorphically-expressed cytochrome P₄₅₀ isoforms in a mammalian subject include, but are not limited to, CYP2C8, CYP2C9, CYP2C19, and CYP2D6.

In certain embodiments, the decrease in metabolism of the compound of Formula 1 by at least one polymorphically-expressed cytochrome P₄₅₀ isoforms cytochrome P₄₅₀ isoform is greater than about 5%, greater than about 10%, greater than about 20%, greater than about 30%, greater than about 40%, or greater than about 50% as compared to the corresponding non-isotopically enriched compound.

The metabolic activities of the cytochrome P₄₅₀ isoforms are measured by the method described in Example 1.

Provided herein are methods for treating a subject, including a human, having or suspected of having a disease involving hypertension, cardiac failure, prostatitis, and/or benign prostatic hyperplasia, or for preventing such disease in a subject prone to the disease; comprising administering to the subject a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; so as to affect at least one statistically-significantly improved disease-control and/or disease-eradication endpoint, as compared to the corresponding non-isotopically enriched compound.

Examples of improved disease-control and/or disease-eradication endpoints include, but are not limited to, statistically-significant improvement in chronotropic, inotropic, vasodilation, and reduction of fibrillation, as compared to the corresponding non-isotopically enriched compound.

Provided herein are methods for treating a subject, including a human, having or suspected of having a disease involving hypertension, cardiac failure, prostatitis, and/or benign prostatic hyperplasia, or for preventing such disease in a subject prone to the disease; comprising administering to the subject a therapeutically effective amount of a compound of Formula 1, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof; so as to affect an improved clinical effect as compared to the corresponding non-isotopically enriched compound. Examples of improved disease-control and/or disease-eradication endpoints include, but are not limited to, statistically-significant improvement in chronotropic, inotropic, vasodilation, or reduction of fibrillation, as compared to the corresponding non-isotopically enriched compound.

In some embodiments, the disease or condition in which it is beneficial to modulate an alpha-adrenergic receptor is selected from the group consisting of hypertension, cardiac failure, prostatitis, and benign prostatic hyperplasia.

In some embodiments, there are provided methods for treating a mammalian subject, particularly a human having, suspected of having, or being prone to a disease or condition in which it is beneficial to modulate an alpha-adrenergic receptor comprising administering to a mammalian subject in need thereof a therapeutically effective amount of an alpha adrenoceptor modulator comprising administering to a mammalian subject in need thereof a therapeutically effective amount of a compound of Formula 1, a single enantiomer of Formula 1, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer of Formula 1, a mixture of diastereomers, or a pharmaceutically acceptable salt, solvate, or prodrug thereof; provided that said compound of Formula 1 contains at least one deuterium atom; and provided that deuterium enrichment in said compound of Formula 1 is at least about 1%.

In other embodiments, there are provided methods for treating a mammalian subject, particularly a human having, suspected of having, or being prone to a disease or condition in which it is beneficial to modulate an alpha-adrenergic receptor comprising administering to a mammalian subject in need thereof a therapeutically effective amount of an alpha adrenoceptor modulator comprising administering to a mammalian subject in need thereof a therapeutically effective amount of a compound of Formula 1, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer; provided that said compound of Formula 1 contains at least one deuterium atom; and provided that deuterium enrichment in said compound of Formula 1 is at least about 1%.

In still other embodiments, there are provided methods for treating a mammalian subject, particularly a human having, suspected of having, or being prone to a disease or condition in which it is beneficial to modulate an alpha-adrenergic receptor comprising administering to a mammalian subject in need thereof a therapeutically effective amount of an alpha adrenoceptor modulator comprising at least one of the compounds of Formula 1, or a single enantiomer according to Formula 1, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer according to Formula 1, a mixture of diastereomers, or a pharmaceutically acceptable salt, solvate, or prodrug thereof wherein the compound cannot be

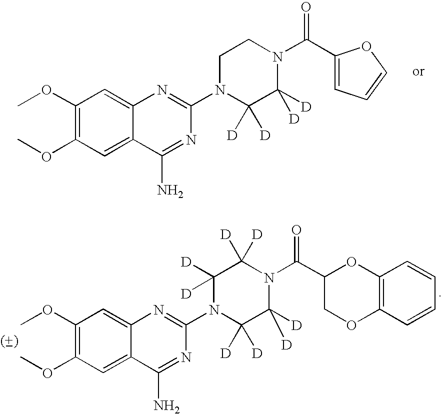

Depending on the disease to be treated and the subject's condition, the compound of Formula 1 provided herein may be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1000 milligrams, from about 0.1 to about 500 milligrams, or from 0.5 about to about 100 milligrams active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Combination Therapy

The compounds provided herein may also be combined or used in combination with other agents useful in the treatment, prevention, or amelioration of one or more symptoms of hypertension, cardiac failure, prostatitis, and/or benign prostatic hyperplasia. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound of Formula 1. When a compound of Formula 1 provided herein is used contemporaneously with one or more other drugs, a pharmaceutical composition containing such other drugs in addition to the compound provided herein may be utilized, but is not required. Accordingly, the pharmaceutical compositions provided herein include those that also contain one or more other active ingredients or therapeutic agents, in addition to the compound provided herein.

In some embodiments, the compounds provided herein can be combined with one or more alpha-adrenergic receptor modulators, including, but not limited to: clonidine, guanethidine, guanfacine, lofexidine, mecamylamine, methyldopa, moxonidine, rescinnamine, reserpine, bosentan, and ketanserin.

In certain embodiments, the compounds provided herein can be combined with one or more calcium channel blockers known in the art, including, but not limited to the group including, amlodipine, diltiazem, felodipine, gallopamil, lacidipine, lercanidipine, menthol, nicardipine, nifedipine, nimodipine, nisoldipine, nitrendipine, and verapamil.

In certain embodiments, the compounds provided herein can be combined with one or more beta adrenergic antagonists (beta blockers) known in the art, including, but not limited to the group including, acebutolol, alprenolol, atenolol, betaxolol, bisoprolol, butoxamine, carvedilol, celiprolol, esmolol, carteolol, dichloroisoprenaline, labetolol, levobunolol, mepindolol, metipranolol, metoprolol, nadolol, nebivolol, oxprenolol, penbutolol, pindolol, practolol, pronethaolol, propranolol, sotalol, and timolol.

In certain embodiments, the compounds provided herein can be combined with one or more nitrates or nitrites known in the art, including, but not limited to the group including glyceryl trinitrate, isosorbide dinitrate, isosorbide mononitrate, pentaerythritol tetranitrate, amyl nitrite, butyl nitrite, isobutyl nitrite, and cyclohexyl nitrite.

The compounds provided herein can also be administered in combination with other classes of compounds, including, but not limited to, endothelin converting enzyme (ECE) inhibitors, such as phosphoramidon; thromboxane receptor antagonists, such as ifetroban; potassium channel openers; thrombin inhibitors, such as hirudin; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-adrenergic agents; beta-adrenergic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

### EXAMPLES

For all of the following examples, standard work-up and purification methods known to those skilled in the art can be utilized. Synthetic methodologies illustrated in Schemes 1-4 are intended to exemplify the applicable chemistry through the use of specific examples and are not indicative of the scope of what is claimed herein.

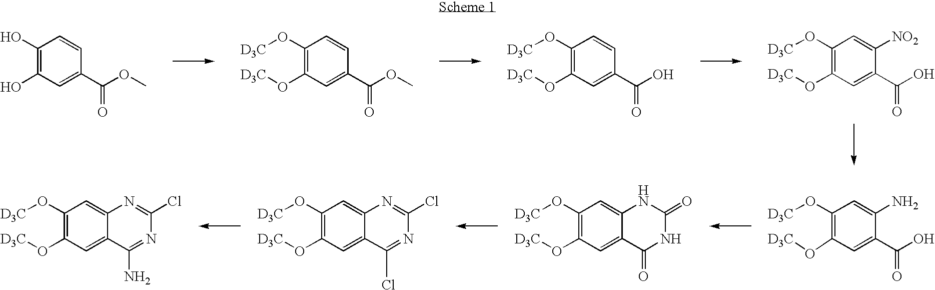

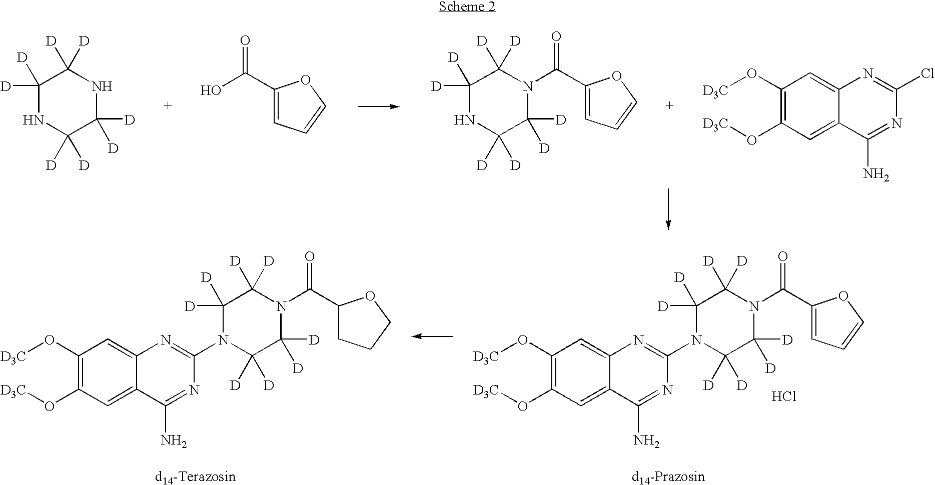

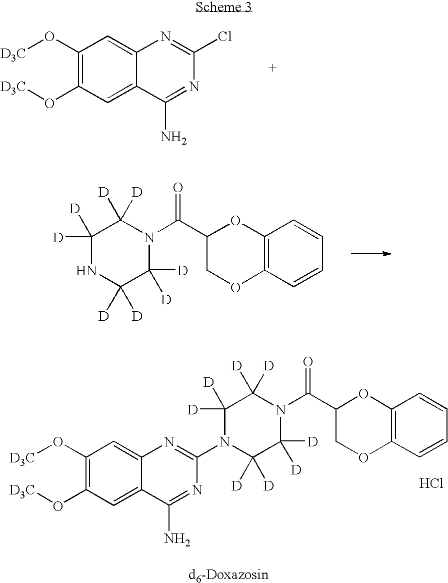

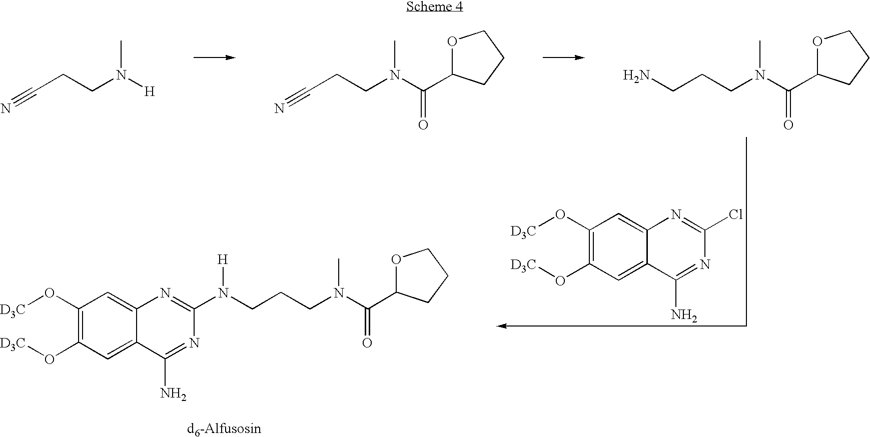

### Example 1

### In vitro Metabolism Using Human Cytochrome P₄₅₀ Enzymes

The cytochrome P₄₅₀ enzymes are expressed from the corresponding human cDNA using a baculovirus expression system (BD Biosciences). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula 1, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g. acetonitrile, 20% trichloroacetic acid, 94% acetonitrile/6% glacial acetic acid, 70% perchloric acid, 94% acetonitrile/6% glacial acetic acid) and centrifuged (10,000 g) for 3 minutes. The supematant is analyzed by HPLC/MS/MS.

Pharmacology

| Cytochrome P₄₅₀ | Standard |
|---|---|
| CYP1A2 | Phenacetin |
| CYP2A6 | Coumarin |
| CYP2B6 | [¹³C]—(S)-mephenytoin |
| CYP2C8 | Paclitaxel |
| CYP2C9 | Diclofenac |
| CYP2C19 | [¹³C]—(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Example 2

### Alpha-Blocker Activity at α₁-Adrenergic Receptors

Radioligand binding assays are performed as previously described in Greengrass et al, European Journal of Pharmacology 1979, 55, 323-326, which is hereby incorporated by reference in its entirety. ³H-Prazosin (33 Ci/mmol) is prepared by reduction of bromoprazosin with tritium gas. Radiochemical purity can be determined by thin-layer chromatography on silica gel in ethyl acetate-methanol-diethylamine (80:20:1) R_{F} 0.7 and ether-isopropylamine (95:5) R_{F} 0.2. The ³H-prazosin stock is stored in ethanol at −20° C. and dilutions to appropriate volumes are made in 0.1% ascorbic acid before use. Male Sprague Dawley Rats (˜200 g) are killed by decapitation and brains (minus cerebella) are rapidly removed and homogenized using a Polytron (setting No. 5, for 20 sec) in 20 volumes (w/v) of ice-cooled 50 millimolar Tris hydrochloride buffer (pH 7.7 at 25° C.). Homegenates are centrifuged twice in an MSE superspeed 65 preparative centrifuge (at 4° C.) for 10 minutes at 50,0000×g, with resuspension of the pellet in fresh buffer between spins. The final pellet is homogenized in 80 volumes (w/v) of ice-cold 50 millimolar Tris hydrochloride buffer (pH 8.0 at 25° C.). Triplicate incubation tubes containing ³H-prazosin, various concentrations of drugs or 0.1% ascorbic acid (50 μl) and an aliquot of freshly resuspended tissue 800 microliters (˜500 g protein) in a final volume of 1 mL. The radioactive ligand and added drugs are all prepared in a 0-1% ascorbic acid solution. The concentration of ascorbic acid (<0.01%) does not influence the binding of ³H-prazosin. The final concentration of ³H-prazosin for saturation assays is (0.05-5 nM) and for competition studies is 0.2 nM. Tubes are incubated at 25° C. for 30 minutes and the incubation is terminated by rapid filtration under vacuum through GF/B glass fiber filters. The filters are rinsed with three 5 mL washes of ice cold 50 millimolar Tris buffer (pH 7.7 at 25° C.). The filters are placed in vials containing 10 mL of Instagel®, cooled overnight, and counted in a Searle Mark 3 liquid scintillation counter at 45% efficiency. Specific binding is defined as the excess over blanks containing 2.0 micromolar phentolamine or 100 nanomolar prazosin and is typically about 90% of the total binding for low ligand concentrations.

### Example 3

### d₆-3,4-Dimethoxybenzoic Acid Methyl Ester

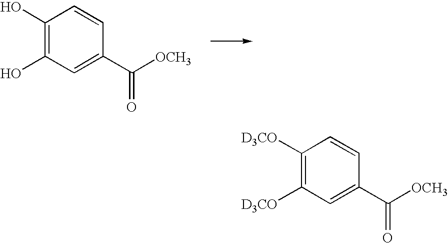

The reaction is performed as previously described Ramaswamy et al, Environ. Sci. Technol. 1985, 19, 507-512, which is hereby incorporated by reference in its entirety. A mixture of 3,4-dihydroxybenzoic acid (1 mmol) in 2 mL of dichloromethane, d₆-dimethylsulfate or d₃-iodomethane (1.5 equiv), and tetra-n-butylammonium hydroxide (1.5 equiv, aqueous 1.5 M solution) is stirred vigorously at room temperature, overnight. The reaction mixture is extracted with 40 mL of ethyl acetate. The extract is then washed 3 times with 10 mL portion of water and dried over anhydrous Na₂SO₄. The solvent is evaporated, and the residue is purified by silica gel chromatography (25% ethyl acetate in hexane) to give the desired product, d₆-3,4-Dimethoxybenzoic acid methyl ester.

### Example 4

### d₆-4,5-Dimethoxy-2-nitrobenzoic acid methyl ester

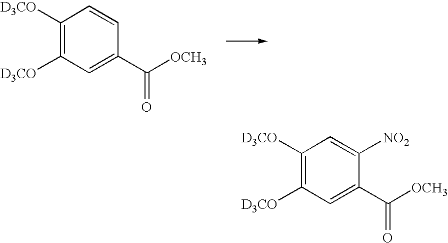

The reaction is performed as previously described U.S. Pat. No. 3,954,748, which is hereby incorporated by reference in its entirety. d₆-3,4-dimethoxybenzoic acid methyl ester (144 g, 0.74 mole is added to 560 mL of 40% nitric acid.), After addition is complete, the mixture is stirred at ambient temperature until a thick paste is obtained. The mixture is then allowed to stand overnight, after which the mixture is diluted with a small amount of water, filtered and washed with water until neutral. The resulting solid is dried under reduced pressure to give the desired product, d₆-4,5-dimethoxy-2-nitrobenzoic acid methyl ester.

### Example 5

### d₆-4,5-Dimethoxy-2-nitrobenzoic acid

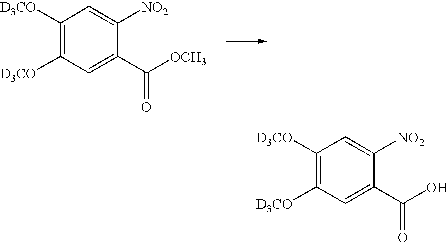

The reaction is performed as previously described in Khurana et al, Monatshefte fur Chemie 2004, 135, 83-87, which is hereby incorporated by reference in its entirety. A mixture of KOH, d₆-4,5-dimethoxy-2-nitrobenzoic acid methyl ester and methanol are stirred at ambient temperature until all starting material is consumed. The mixture is then extracted with ether, and the aqueous layer is acidified with 6N HCl. In the next step, the aqueous layer is extracted with ethyl acetate, dried over anhydrous Na₂SO₄. The solvent is evaporated to give the desired product, d₆-4,5-dimethoxy-2-nitrobenzoic acid.

### Example 6

### d₆-4,5-Dimethoxy-2-aminobenzoic acid

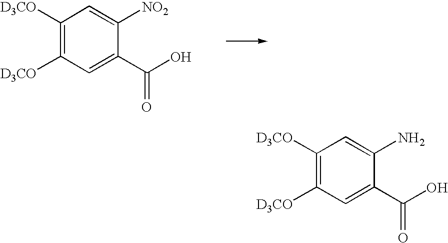

The reaction performed as previously described Andrus et al, Journal of Organic Chemistry 2002, 67(23), 8284-8286, which is hereby incorporated by reference in its entirety. A mixture of d₆-4,5-dimethoxy-2-nitrobenzoic acid (5 g, 22 mmol) and 10% Raney nickel in 25 mL of 2-propanol is heated at 45° C. under H₂ atmosphere (80 bar). After 5 hours, the reaction mixture is filtered, dried under vacuum and recrystallized from ethanol to give the desired product, d₆-4,5-dimethoxy-2-aminobenzoic acid.

### Example 7

### d₆-6,7-Dimethoxy-2,4-quinazolinedione

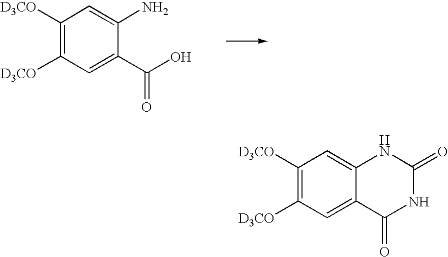

The reaction performed as previously described Andrus et al. A solution of sodium cyanate (2.4 g, 36.9 mmol) in water (10 mL) is slowly added to a stirred suspension of d₆-4,5-dimethoxy-2-aminobenzoic acid (2.48 g, 15 mmol) in a mixture of water (90 mL) and glacial acetic acid (1.5 mL) at 35° C. After addition is complete, the reaction mixture is stirred for 30 minutes. Sodium hydroxide (26.6 g, 0.67 mol) is then added in small portions to the suspension to give a colorless precipitate. After cooling to ambient temperature, the pH of the suspension is adjusted to pH 4 with concentrated HCl. The precipitate is filtered, washed thoroughly with water and dried at 100° C. to give the desired product, d₆-6,7-dimethoxy-2,4-quinazolinedione.

### Example 8

### d₆-2,4-Dichloro-6,7-dimethoxyquinazoline

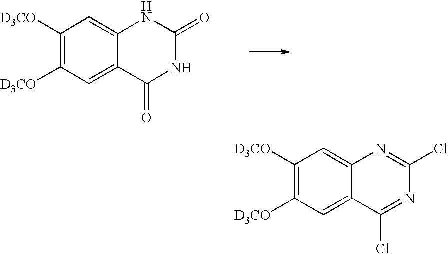

The reaction is performed as previously described Andrus et al. A mixture of d₆-6,7-dimethoxy-2,4-quinazolinedione (3.34 g, 15.05 mmol), 10 mL (0.107 mol) of POCl₃ and 1 mL (12.8 mmol) of N,N-dimethylaniline is refluxed for 4.5 hours, cooled, and allowed to stir overnight at ambient temperature. The mixture is then added to 70 mL of ice water, and the resulting precipitate is filtered, washed with water and dried to give the desired product, d₆-2,4-dichloro-6,7-dimethoxyquinazoline.

### Example 9

### d₆-2-Chloro-4-amino-6,7-dimethoxyquinazoline

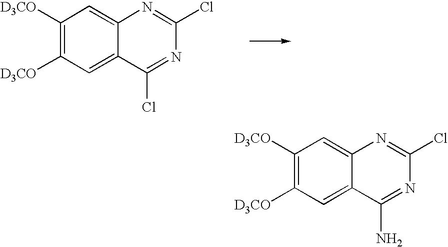

The reaction is performed as previously described Andrus et al. A solution of d₆-2,4-dichloro-6,7-dimethoxyquinazoline (15 g, 8 mmol) in 400 mL of THF, is saturated with anhydrous NH₃, and stirred for 44 hours at ambient temperature. The resulting precipitate is collected and recrystallized from methanol to give the desired product, d₆-2-chloro-4-amino-6,7-dimethoxyquinazoline.

### Example 10

### d₈-Furan-2-yl-piperazin-1-yl-methanone

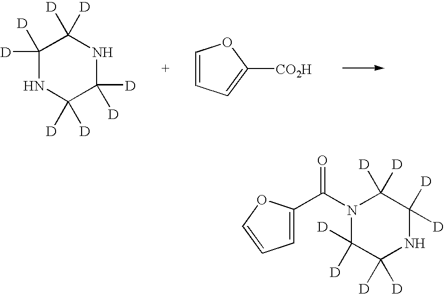

The reaction is performed as previously described Bandgar et al, Synthetic Communications 2004, 34(16), 2917-2924, which is hereby incorporated by reference in its entirety. A mixture of furancarboxylic acid (1 mmol) and triphenylphosphine (2 mmol) in dichloromethane (10 mL) is cooled to 0° C. NBS (2.5 mmol) is added and the reaction mixture is stirred for 15 min. A mixture of d₈-piperazine (C/D/N isotopes, 1 mmol) and pyridine (2.5 mmol) is then added and the resulting mixture is stirred at ambient temperature until the reaction is completed. The solvent is then removed, the residue is extracted with n-hexane, washed with water (10 mL) and aq. sodium bicarbonate (10%, 10 mL). The crude product, d₈-furan-2-yl-piperazin-1-yl-methanone, is further purified by column chromatography.

### Example 11

### d₈-(2,3-Dihydro-benzo[1,4]dioxin-2-yl)-piperazin-1-yl-methanone

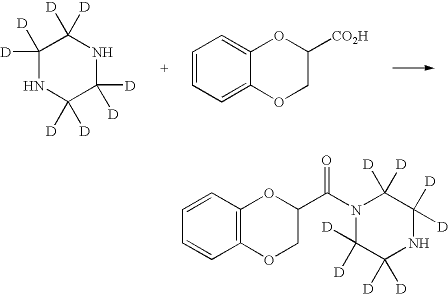

The reaction performed as described in Example 10.

### Example 12

### d₁₄-[4-(4-Amino-6,7-dimethoxy-quinazolin-2-yl)-piperazin-1-yl]-(2,3-dihydro-benzo[1,4]dioxin-2-yl)-methanone hydrochloride salt (d₁₄-Doxazosin hydrochloride salt)

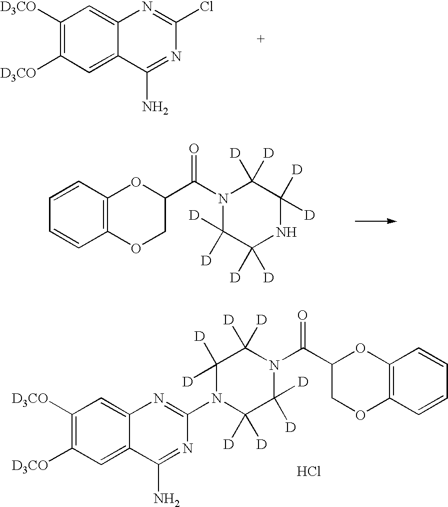

The reaction is performed as previously described Campbell et al, Journal of Medicinal Chemistry 1987, 30, 49-57, which is hereby incorporated by reference in its entirety. A mixture of d₈-(2,3-Dihydro-benzo[1,4]dioxin-2-yl)-piperazin-1-yl-methanone (1.1 equiv) and d₆-2-chloro-4-amino-6,7-dimethoxyquinazoline (1 equiv) in 1-butanol is heated to reflux and the resulting white precipitate is filtered and recrystallized to yield the desired product, d₁₄-doxazosin hydrochloride salt).

### Example 13

### d₁₄-[4-(4-Amino-6,7-dimethoxy-quinazolin-2-yl)-piperazin-1-yl]-furan-2-yl-methanone hydrochloride salt (d₁₄-Prazosin hydrochloride salt)

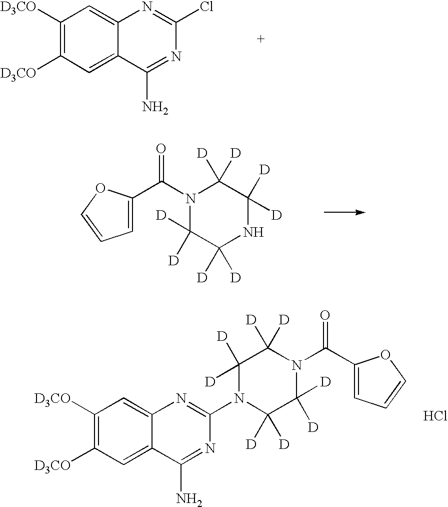

The reaction is performed as previously described Althuis et al, Journal of Medicinal Chemistry 1976, 20(1), 146-9, which is hereby incorporated by reference in its entirety. A mixture of d₈-furan-2-yl-piperazin-1-yl-methanone (1.1 equiv) and d₆-2-chloro-4-amino-6,7-dimethoxyquinazoline (1 equiv) in isoamyl alcohol is heated and the resulting white precipitate is filtered and recrystallized to yield the desired product, d₁₄-prazosin hydrochloride salt.

### Example 14

### d₁₄-[4-(4-Amino-6,7-dimethoxy-quinazolin-2-yl)-piperazin-1-yl]-(tetrahydro-furan-2-yl)-methanone (d₁₄-Terazosin)

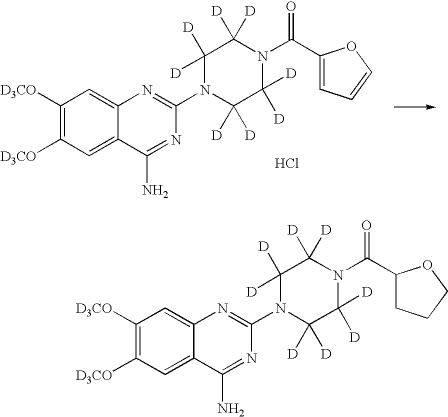

The reaction performed as previously described Egan et al, Synthetic Communications 2004, 34(10), 1881 -1884, which is hereby incorporated by reference in its entirety. A solution of d₁₄-Prazosin hydrochloride salt in ethanol:water (8:1) is reduced with H₂ gas using 5% Pd/C catalyst with rapid stirring at ambient temperature overnight. The reaction is filtered and the catalyst is washed with ethanol. The crude residue is purified by silica gel chromatography (ethyl acetate-methanol-triethylamine, 70:20:5) to give d₁₄-Terazosin.

### Example 15

### Tetrahydro-N-(3-cyanopropyl)-N-methylfurancarboxamide

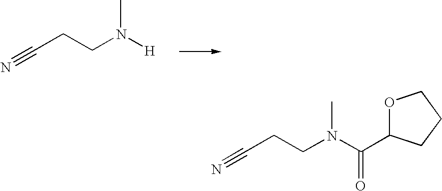

The reaction is performed as previously described Manoury et al, Journal of Medicinal Chemistry 1986, 29, 19-25, which is hereby incorporated by reference in its entirety. A solution of 34.8 g (0.3 mol) of tetrahydro-2-furancarboxylic acid and 30.3 g of Et₃N in 250 mL of THF is added drop wise at 0° C., to 32.4 g (0.3 mol) of ethyl chloroformate. During the addition, the temperature of the mixture is kept below 5° C. The mixture is then stirred for 15 min at 5° C., and a solution of 25.2 g (0.3 mol) of 3-(methylamino)-propanenitrile in 100 mL of THF is slowly added. The mixture is then stirred for 1 hour at 5° C., allowed to warm to ambient temperature, and stirring kept overnight. The mixture is then filtered off and the solvent evaporated. The residual liquid is distilled to give the desired product, tetrahydro-N-(3-cyanopropyl)-N-methylfurancarboxamide.

### Example 16

### Tetrahydro-N-[3-(methylamino)propyl]-2-furancarboxamide

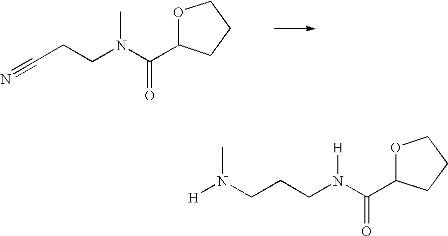

The reaction is performed as previously described Manoury et al. A solution of 26.5 g (0.145 mol) of tetrahydro-N-(3-cyanopropyl)-N-methylfurancarboxamide in 300 mL of 10% ethanolic ammonia is hydrogenated at 80° C. over Rh/C at 840 psi hydrogen pressure. After H₂ uptake is complete, the mixture is cooled, the catalyst filtered off and the filtrate is concentrated to afford the desired product, tetrahydro-N-[3-(methylamino)propyl]-2-furancarboxamide.

### Example 17

### d₆-N-[3-[(4-Amino-6,7-dimethoxy-2-quinazolinyl)methylamino]-propyl]tetrahydro-2-furancarboxamide hydrochloride salt (d₆-Alfusosin hydrochloride salt)

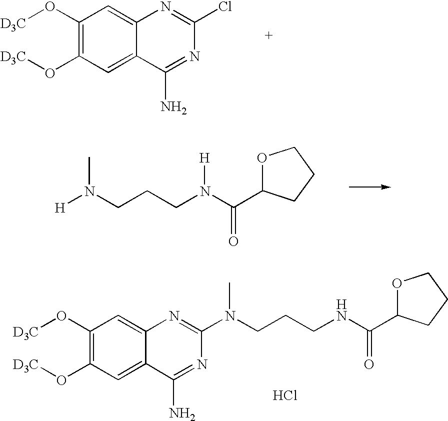

The reaction is performed as previously described Manoury et al. A mixture of 18.4 g (0.1 mol) of tetrahydro-N-[3-(methylamino)propyl]-2-furancarboxamide and 21.5 g (0.09 mol) of d₆-2-chloro-4-amino-6,7-dimethoxyquinazoline in 250 mL of isoamyl alcohol is heated to reflux, under argon for 12 hours. The mixture is cooled, and the precipitate is filtered off and washed with isoamyl alcohol and ethyl ether. The filtrate is concentrated in vacuo. The residue is titrated in acetone and filtered off. The crude solid is recrystallized to give the desired product, d₆-alfusosin hydrochloride salt.

### Example 18

### d₆-3,4-Dimethoxybenzoic acid methyl ester

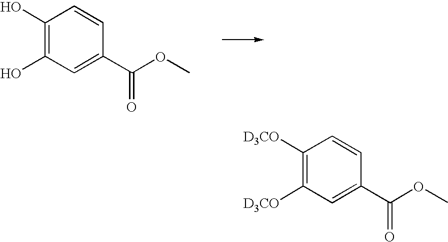

3,4-Dihydroxybenzoic acid methyl ester (9.36 g, 0.056 mol) was added to a suspension of sodium hydride (6.6 g, 0.168 mol) in dry N,N-dimethylformamide (200 mL) at 0° C. The mixture was stirred for 1 hour, d₃-methyl methanesulfonate (2.5 equiv) was added dropwise and stirring was maintained at ambient temperature overnight. The reaction was quenched with water and extracted with ethyl acetate. The combined organic layers were washed with water, brine, dried and concentrated to yield a yellow oil. The crude residue was purified by column chromatography to give the desired product, d₆-3,4-dimethoxybenzoic acid methyl ester (4.57 g, 40%). ¹H NMR (300 MHz, CDCl₃) δ 7.71 (dd, J=8.4, 2.1 Hz, 1H), 7.55 (d, J=2.1 Hz, 1H), 6.94 (d, J=8.4 Hz, 1H), 3.9 (s, 3H), MS (EI⁺) 203.

### Example 19

### d₆-4,5-Dimethoxy-2-nitrobenzoic acid methyl ester

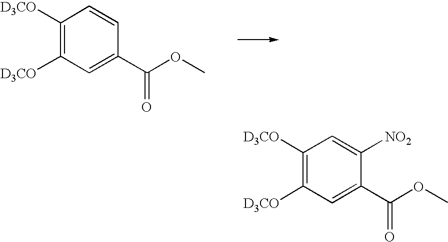

d₆-3,4-Dimethoxybenzoic acid methyl ester (4.57 g, 0.023 mol) was added to 15 mL of 65% nitric acid. The mixture was stirred at ambient temperature overnight, filtered, the solid was washed with water until neutral, and dried to give the desired product, d₆-4,5-dimethoxy-2-nitrobenzoic acid methyl ester. (4.5 g, 78% yield). ¹H NMR (300 MHz, DMSO-d₆) δ 7.64(s, 1H), 7.32 (s, 1H), 3.83 (s, 3H), MS (EI⁺) 248.

### Example 20

### d₆-4,5-Dimethoxy-2-nitrobenzoic acid

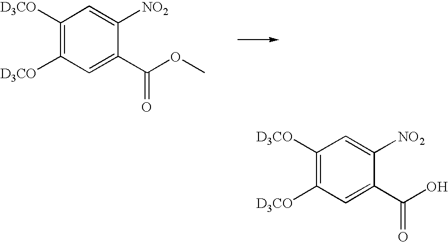

A mixture of potassium hydroxide (3.07 g, 55 mmol), d₆-4,5-dimethoxy-2-nitro-benzoic acid methyl ester (4.5 g, 18 mmol) and methanol (50 mL) were stirred at 55° C. overnight. The mixture was acidified with 3N hydrochloric acid, filtered, washed with water and ethyl ether, and dried to afford the title product as a yellow solid (3.6 g, 86%). ¹H NMR (300 MHz, DMSO-d₆) δ 13.57 (s, 1H), 7.56 (s, 1H), 7.28 (s, 1H), ESI-MS m/z 232 (M−H).

### Example 21

### d₆-4,5-Dimethoxy-2-aminobenzoic acid hydrochloride salt

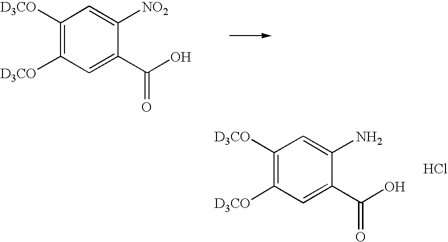

A mixture of d₆-4,5-dimethoxy-2-nitrobenzoic acid (3.6 g, 15.5 mmol) and 10% Pd on carbon in 80 mL of methanol was hydrogenated under hydrogen atmosphere for 5 hours. The catalyst was filtered, A solution of hydrochloric acid in methanol was added, the solvent was removed and the crude residue was recrystallized from methanol to give the desired product, d₆-4,5-dimethoxy-2-aminobenzoic acid (2.29 g, 61%). ¹H NMR (300 MHz, DMSO-d₆) δ 7.18 (s, 1H), 6.45 (s, 1H), ESI-MS m/z 204 (M+H).

### Example 22

### d₆-6,7-Dimethoxy-2,4-quinazolinedione

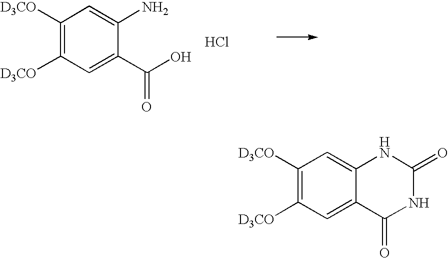

d₆-4,5-dimethoxy-2-aminobenzoic acid hydrochloride salt (0.78 g, 3.3 mmol) was dissolved in water (23 mL) and the pH was adjusted to 4 with sodium hydroxide. Acetic acid (0.36 mL) and a solution of sodium cyanate (0.47 g, 7.26 mmol) in water (2.5 mL) were added, and he reaction was kept at 35° C. for 3 hours. Sodium hydroxide was added in small portions, and the mixture was heated to 90° C. for 1 hour, cooled and the pH was adjusted to 4 with concentrated hydrochloric acid. The precipitate was filtered, washed with water and dried to give the desired product as a white solid (0.76 g, 100%). ¹H NMR (300 MHz, DMSO-d₆) δ11.08 (br. s, 1H), 10.91 (br. s, 1H), 7.258 (s, 1H), 6.68 (s, 1H); ESI-MS m/z 229 (M+H).

### Example 23

### d₆-2,4-Dichloro-6,7-dimethoxyquinazoline

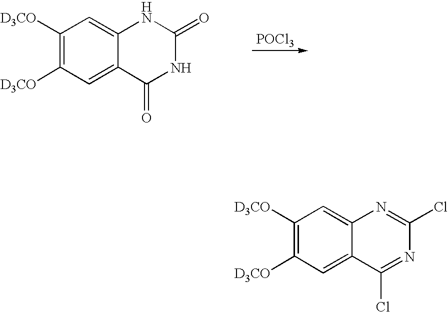

A mixture of d₆-6,7-dimethoxy-2,4-quinazolinedione (0.76 g, 3.33 mmol), 4.2 mL of phosphorus oxychloride and 0.22 mL of N,N-dimethylaniline was heated to reflux for 4 hours, cooled, and allowed to stir overnight at ambient temperature. The mixture was poured into ice water, and the resulting precipitate was filtered, washed with water and dried to give the desired product, d₆-2,4-dichloro-6,7-dimethoxy-quinazoline (0.81 g, 92%). ¹H NMR (300 MHz, CDCl₃) δ 7.37 (s, 1H), 7.29(s, 1H); ESI-MS m/z 266 (M+H).

### Example 24

### d₆-2-Chloro-4-amino-6,7-dimethoxyquinazoline

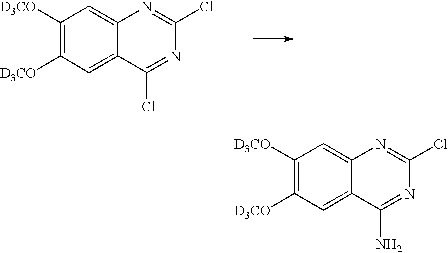

A solution of d₆-2,4-dichloro-6,7-dimethoxyquinazoline (810 mg, 3 mmol) in 25 mL of tetrahydrofuran was saturated with anhydrous ammonia and stirred for 40 hours at ambient temperature in a sealed tube. The resulting precipitate was collected, washed with ethyl ether and dried to yield the desired product, d₆-2-chloro-4-amino-6,7-dimethoxyquinazoline (440 mg, 59%). ¹H NMR (300 MHz, DMSO-d₆) δ 7.91 (s, 1H), 7.57 (s, 1H), 7.04 (s, 1H); ESI-MS m/z 246 (M+H).

### Example 25

### d₆-[4-(4-Amino-6,7-dimethoxy-quinazolin-2-yl)-piperazin-1-yl]-furan-2-yl-methanone hydrochloride salt (d₆-prazosin hydrochloride salt)

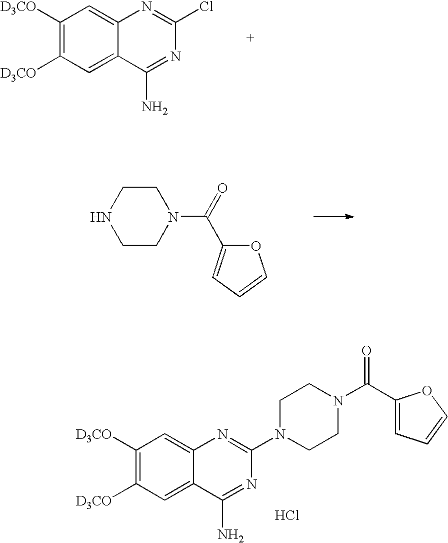

A mixture of furan-2-yl-piperazin-1-yl-methanone (198 mg, 1.1 mmol) and d₆-2-chloro-4-amino-6,7-dimethoxyquinazoline (246 mg, 1 mmol) in isoamyl alcohol (25 mL) was heated at reflux overnight. The resulting white precipitate was filtered and dried to yield the desired product, d₆-prazosin hydrochloride salt. ¹H NMR (300 MHz, D₂O-DMSO-d₆) δ12.35 (br. s, 1H), 7.79 (s, 1H), 7.53 (s, 1H), 7.03 (d, J=3.6 Hz, 1H), 7.01 (d, J=3.6 Hz, 1H), 6.61 (s 1H), 3.90-3.81 (m, 8H); ESI-MS m/z 390 (M+H).

### Example 26

### d₆-[4-(4-Amino-6,7-dimethoxy-quinazolin-2-yl)-piperazin-1-yl]-(tetrahydro-furan-2-yl)-methanone (d₆-terazosin hydrochloride salt)

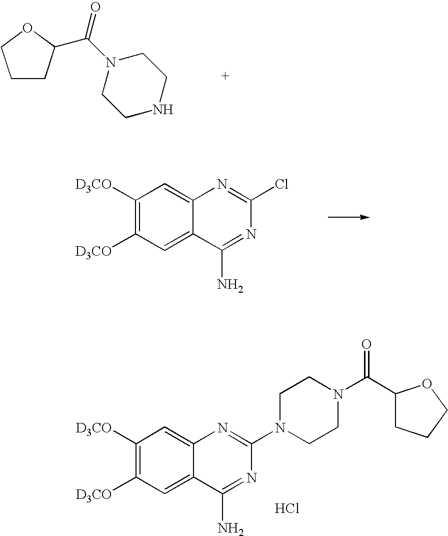

A mixture of tetrahydrofuran carboxylic acid (1.16 g, 10 mmol), piperazine (0.86 g, 10 mmol) and hexamethyldisilazane (1.61 g, 10 mmol) was heated to 110° C. for 15 hours. The reaction was cooled, concentrated, and purified by column chromatography to yield tetrahydrofuran-2-yl-piperazin-1-yl-methanone (729 mg). ¹H NMR (300 MHz, CDCl₃) δ 4.61 (dd, J=7.2, 5.4 Hz, 1H), 3.97 (m, 1H), 3.87 (m, 1H), 3.66 (m, 2H), 3.48 (m, 2H), 2.83-2.92 (m, 4H), 2.41 (br. s, 1H), 2.23 (m, 1H), 1.84-2.21 (n, 3H); ESI-MS m/z 185 (M+H).

A mixture of tetrohydrofuran-2-yl-piperazin-1-yl-methanone (245 mg, 1.0 mmol) and d₆-2-chloro-4-amino-6,7-dimethoxyquinazoline (184 mg, 1.0 mmol) in isoamyl alcohol (25 mL) was heated at reflux overnight. The resulting white precipitate was filtered and dried to yield the desired product, d₆-terazosin hydrochloride salt. ¹H NMR (300 MHz, DMSO-d₆) δ 12.22 (br. s, 1H), 8.86 (br. s, 1H), 8.67 (br. s, 1H), 7.71 (s, 1H), 7.42 (br. s, 1H), 4.715 (dd, J=7.2, 5.4 Hz, 1H), 3.91-3.62 (m, 10H), 2.03 (m, 2H), 1.83 (m, 2H); ESI-MS m/z 394 (M+H).

### Example 27

### d₆-[4-(4-Amino-6,7-dimethoxy-quinazolin-2-yl)-piperazin-1-yl]-(2,3-dihydro-benzo1,4]dioxin-2-yl)-methanone hydrochloride salt

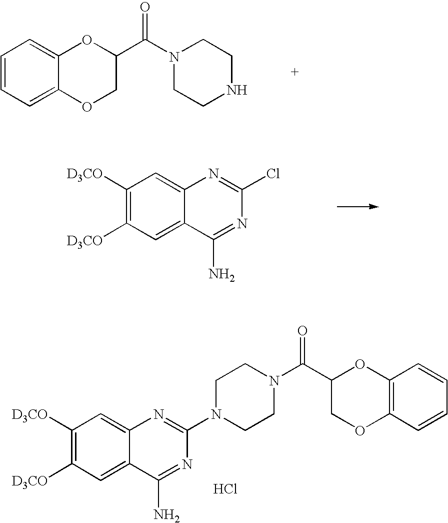

A mixture of 1,4-benzodioxane-6-carboxylic acid (901 mg, 5 mmol), piperazine (431 mg, 5 mmol) and hexamethyldisilazane (1.61 g, 10 mmol) was heated to 110° C. for 15 hours. The reaction was cooled, concentrated, and purified by column chromatography to yield 2,3-dihydro-benzo[1,4]dioxin-2-yl)-piperazin-1-yl-methanone (400 mg, 32%). ¹H NMR (300 MHz, CDCl₃) δ 6.94-6.85 (m, 4H), 4.84 (dd, J=8.1, 2.4 Hz, 1H), 4.52 (dd, J=12.0, 2.4 Hz, 1H), 4.330 (dd, J=12.0, 8.1 Hz, 1H), 3.75 (m, 2H), 3.56 (m, 2H), 2.99-2.91 (m, 4H); ESI-MS m/z 249 (M+H).

A mixture of (2,3-dihydro-benzo[1,4]dioxin-2-yl)-piperazin-1-yl-methanone (290 mg, 1.2 mmol) and d₆-2-chloro-4-amino-6,7-dimethoxyquinazoline (1 mmol, 245.5 mg) in isoamyl alcohol (25 mL) was heated to reflux overnight and the resulting white precipitate was filtered to yield the desired product (210 mg, 43%). ¹H NMR (300 MHz, DMSO-d₆) δ12.23(br. s, 1H), 8.89 (br. s, 1H), 8.68 (br. s, 1H), 7.73 (s, 1H), 7.46 (s, 1H), 6.92-6.82 (m, 4H), 5.32 (d, J=4.2 Hz, 1H), 4.42 (d, 1H), 4.208 (dd, J=11.7, 6.3 Hz, 1H), 3.94-3.65 (m, 8H); ESI-MS m/z 458 (M+H).

### Example 28

### d₆-3-[(4-Amino-6,7-dimethoxy-quinazolin-2-yl)-methyl-amino]-propionitrile

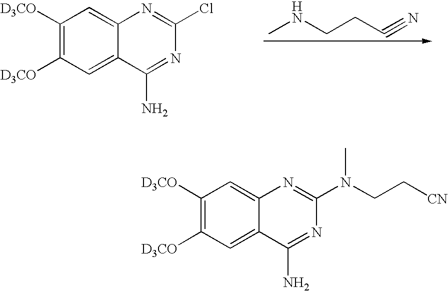

A mixture of d₆-2-chloro-4-amino-6,7-dimethoxyquinazoline (209 mg, 0.85 mmol) and N-(methylamino)-3-propanenitrile (143 mg, 1.7 mmol) in 6 mL of isoamyl alcohol was stirred at reflux under nitrogen for 5 hours. The mixture was cooled, filtered and the solid was washed several times with ethyl ether to afford the title product (120 mg, 43%). ¹H NMR (300 MHz,CD₃OD-d₄) δ7.49 (s, 1H), 7.12 (s, 1H), 3.98 (t, J=6.6 Hz, 2H), 3.252 (s, 3H), 2.84 (t, J=6.6 Hz, 2H),; ESI-MS m/z 294 (M+H).

### Example 29

### N²-(3-Amino-propyl)-6,7-dimethoxy-N²-methyl-quinazoline-2,4-diamine

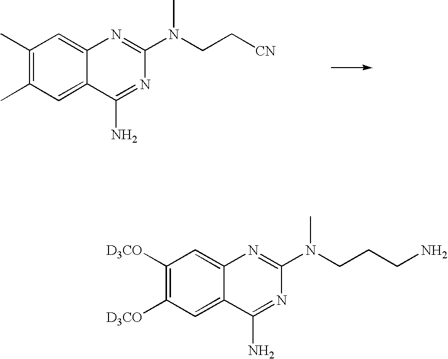

A solution of d₆-[(4-amino-6,7-dimethoxy-2-quinazolinyl)-methylamino]-propanenitrile (360 mg, 1.2 mmol) in methanol was hydrogenated for 24 hours at 50° C. under 50 psi of hydrogen, using 5% rhodium on Al₂O₃. The mixture was cooled, the catalyst was filtered and the solvent was removed to yield a yellow solid which was used directly in the next step. ESI-MS m/z 298 (M+H).

### Example 30

### d₆-N-[3-[(4-Amino-6,7-dimethoxy-2-quinazolinyl)methylamino]-propyl]tetrahydro-2-furancarboxamide

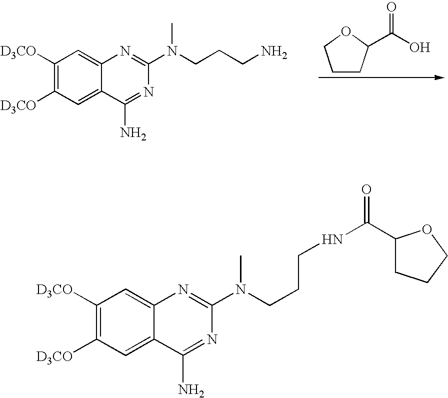

A solution of tetrahydro-2-furoic acid (235 mg, 2.2 mmol) and N,N′-carbonyl diimidazole (357 mg, 2.2 mmol) in 4 mL of N,N-dimethylformamide was stirred for 30 minutes at 45° C. under a nitrogen atmosphere. A solution of d₆-N²-(3-aminopropyl)-6,7-dimethoxy-N²-methyl-quinazoline-2,4-diamine (405 mg, 1.36 mmol) in N,N-dimethylformamide (30 mL) was then added and the mixture was heated to reflux for 2.5 hours. The solvent was evaporated under reduced pressure, the residue was diluted with saturated aqueous sodium bicarbonate aqueous solution, and extracted with dichloromethane. The organic layer was washed with water and brine, dried, and the solvent was evaporated to afford a yellow oil which was purified by preparative HPLC to yield the title product (200 mg, 37%). ¹H NMR (300 MHz,CDCl₃) δ8.32 (br. s, 1H), 7.70 (br. s, 1H), 7.41 (s, 1H), 7.03 (s, 1H), 4.43 (dd, J=7.2, 6.0 Hz,1H), 4.02 (m, 2H), 3.66 (m, 2H), 3.48-3.23 (m, 4H), 3.13 (s, 3H), 2.34 (m, 1H), 2.20-1.88 (m, 3H), 1.744 (s, 1H); ESI-MS m/z 395 (M+H).

The examples set forth above are provided to give those of ordinary skill in the art with a complete disclosure and description of how to make and use the claimed embodiments, and are not intended to limit the scope of what is disclosed herein. All publications, patents, and patent applications cited in this specification are incorporated herein by reference as if each such publication, patent or patent application were specifically and individually indicated to be incorporated herein by reference.

What is claimed is:

1. A compound of Formula 1: 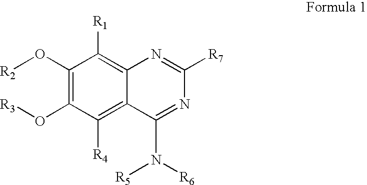
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 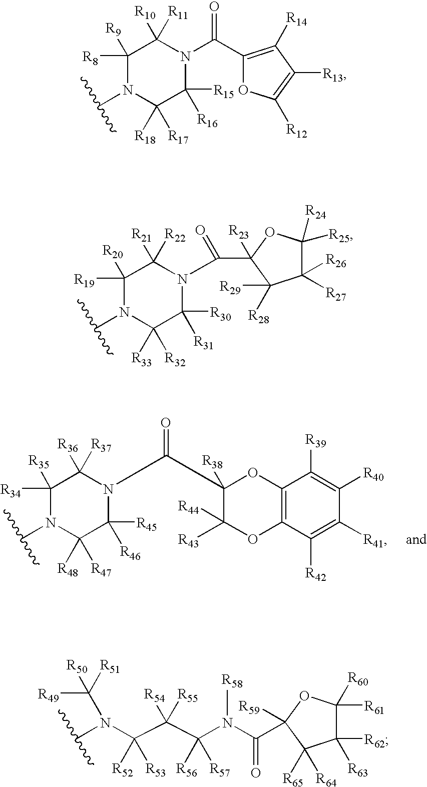
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium; provided that compounds of Formula 1 contain at least one deuterium atom and that deuterium enrichment in compounds of Formula 1 is at least about 1%;
with the proviso that compounds of Formula 1 cannot be selected from the group consisting of:

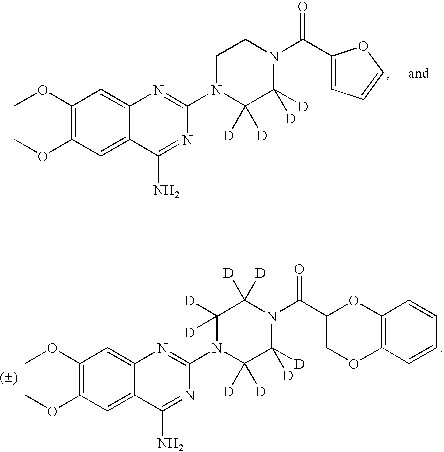

2. The compound of claim 1, wherein said compound contains the mixture of about 90% or more by weight of the (−)-enantiomer and about 10% or less by weight of the (+)-enantiomer.

3. The compound of claim 1, wherein said compound contains the mixture of about 90% or more by weight of the (+)-enantiomer and about 10% or less by weight of the (−)-enantiomer.

4. The compound of claim 1 selected from the group consisting of: 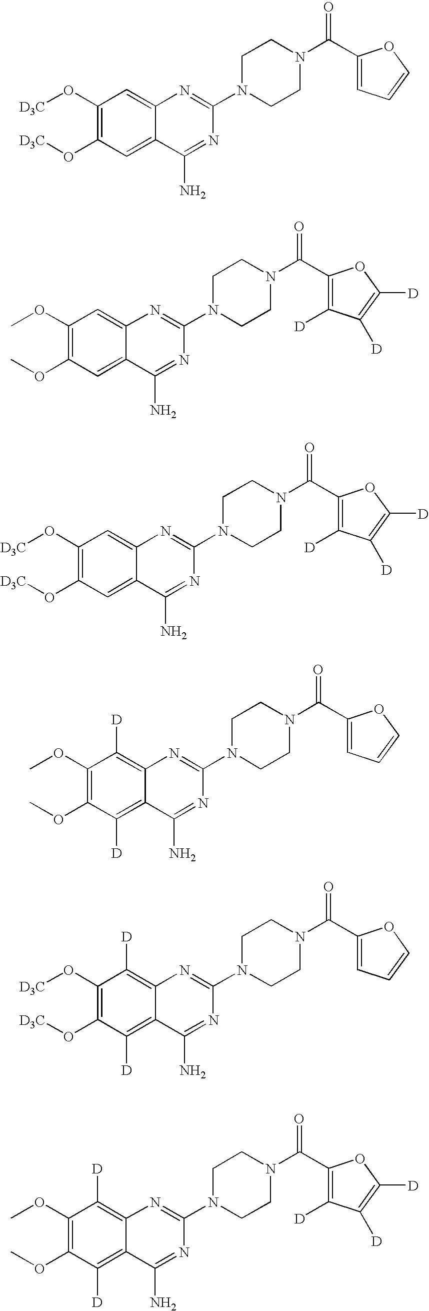 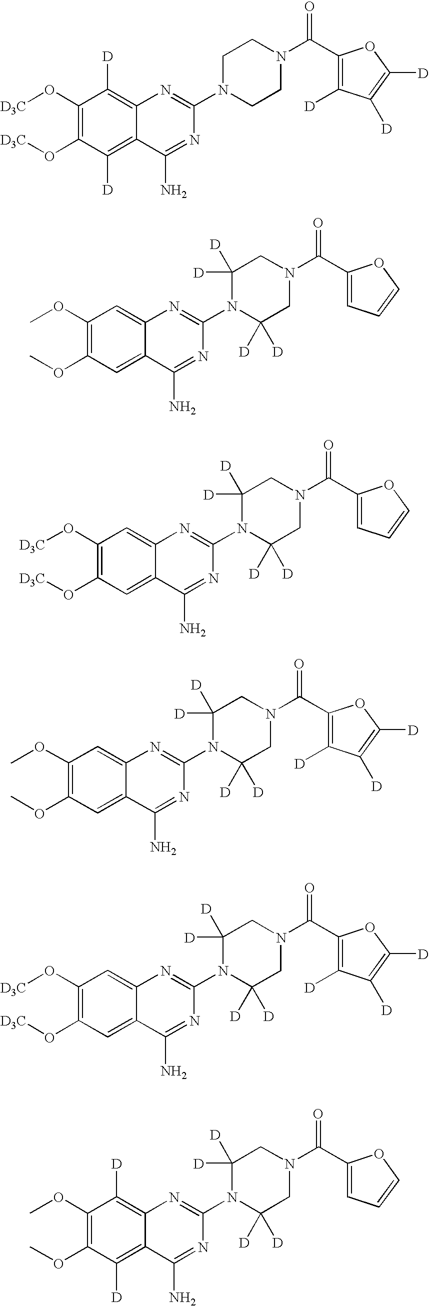 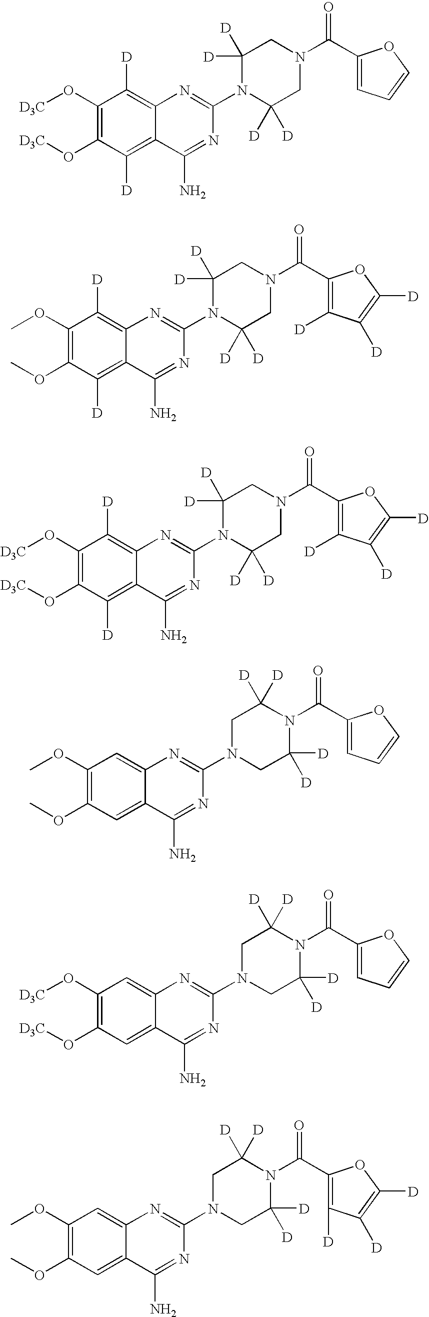 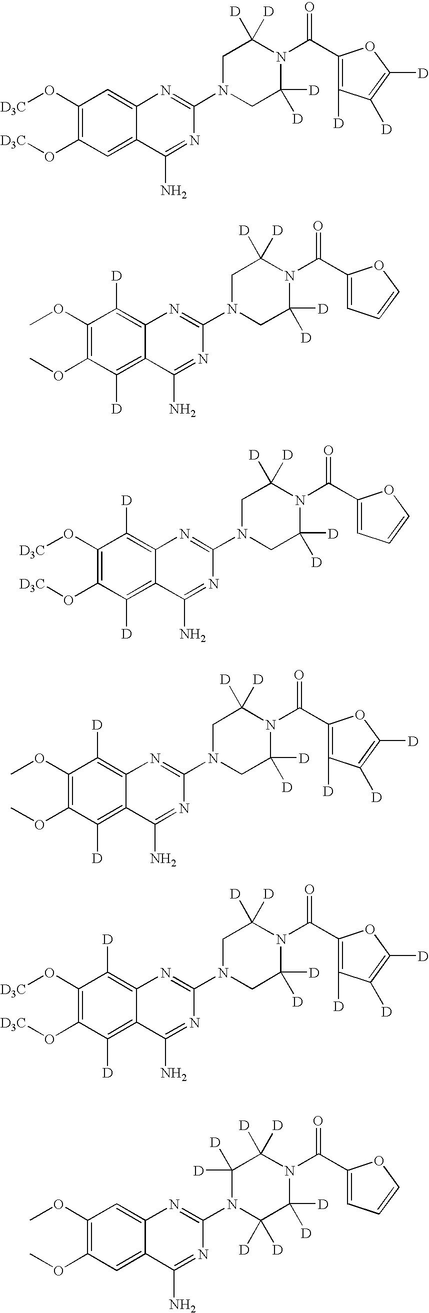 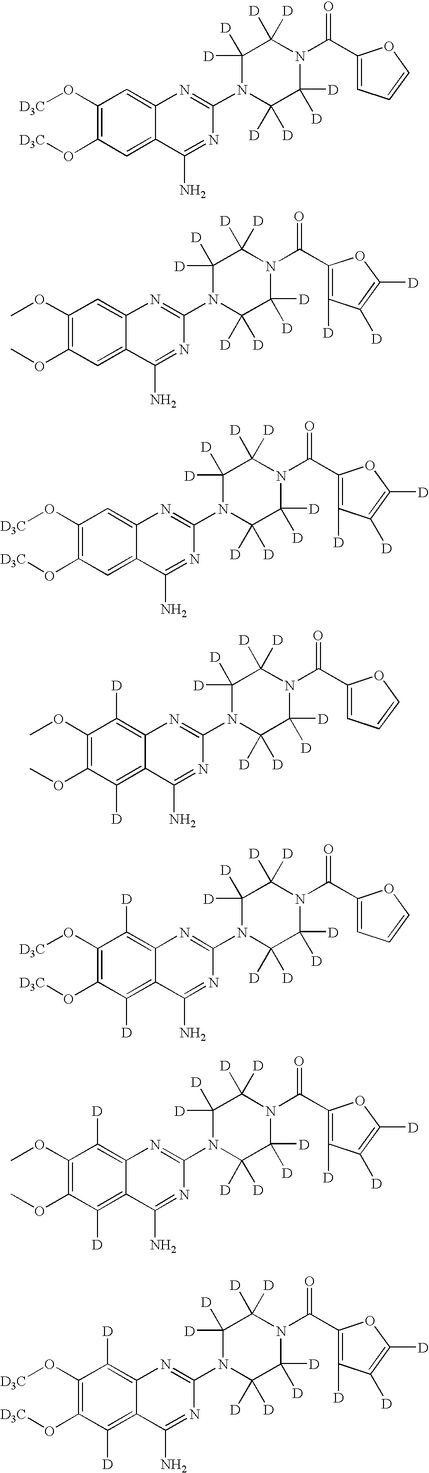 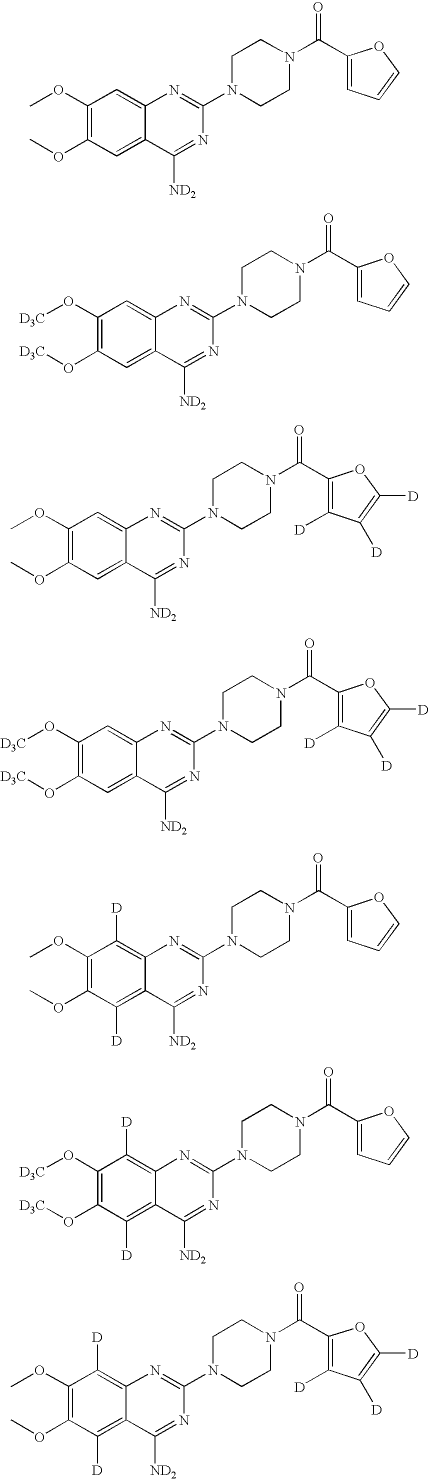 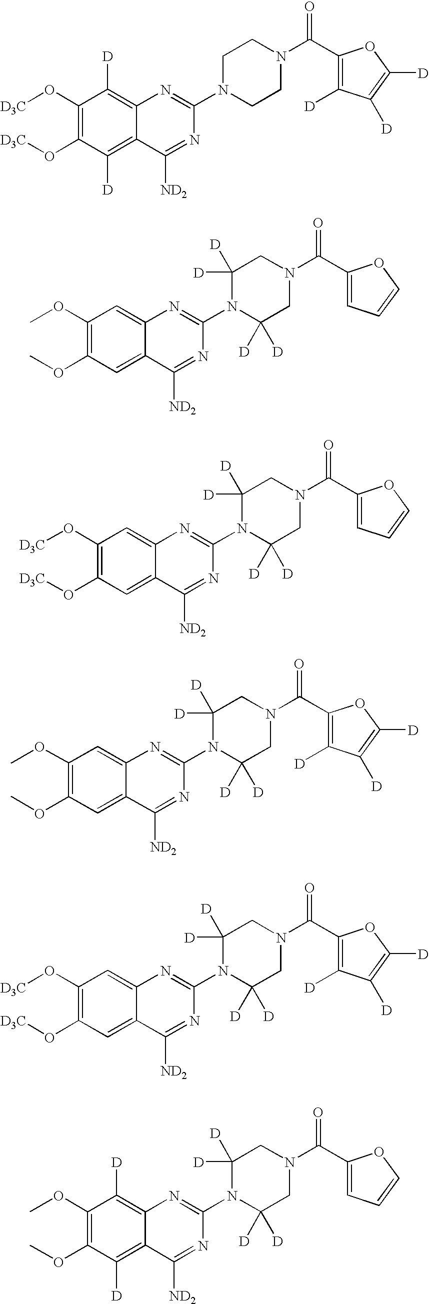 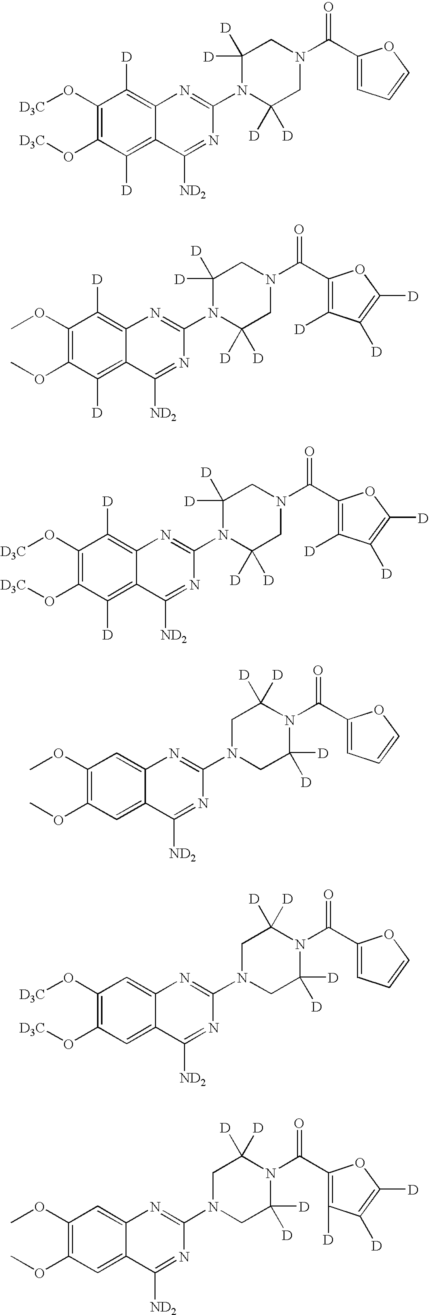 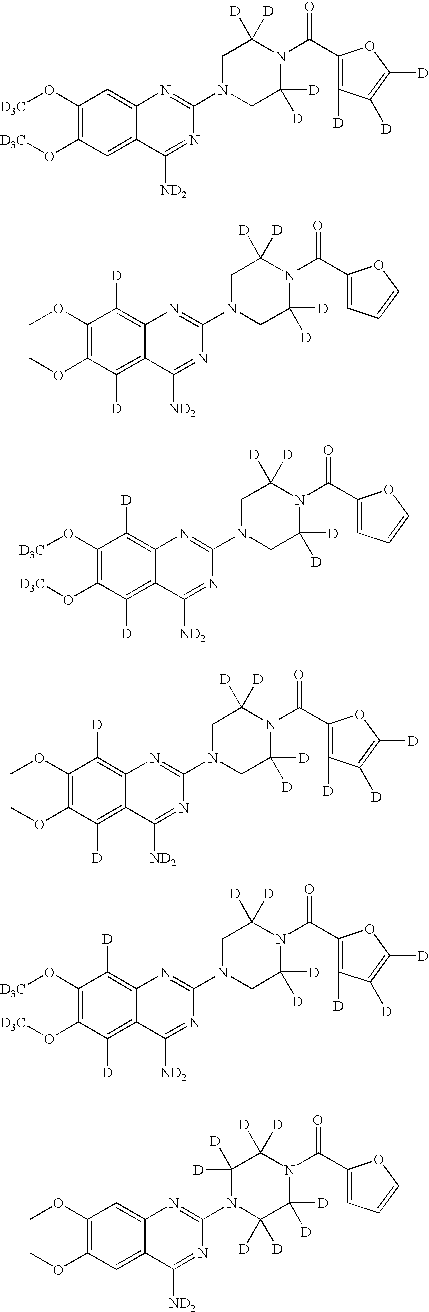 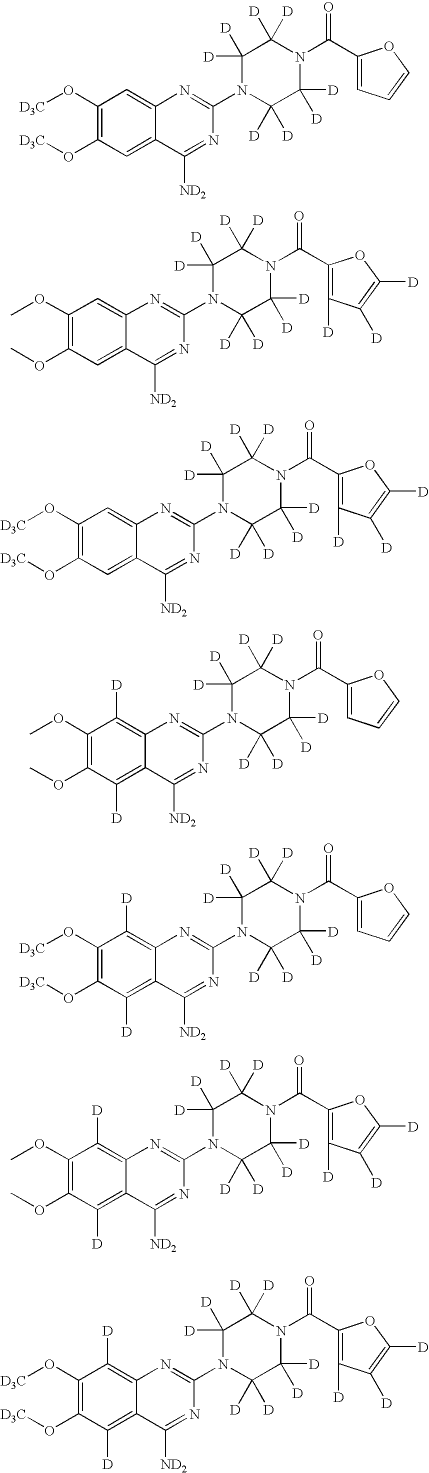 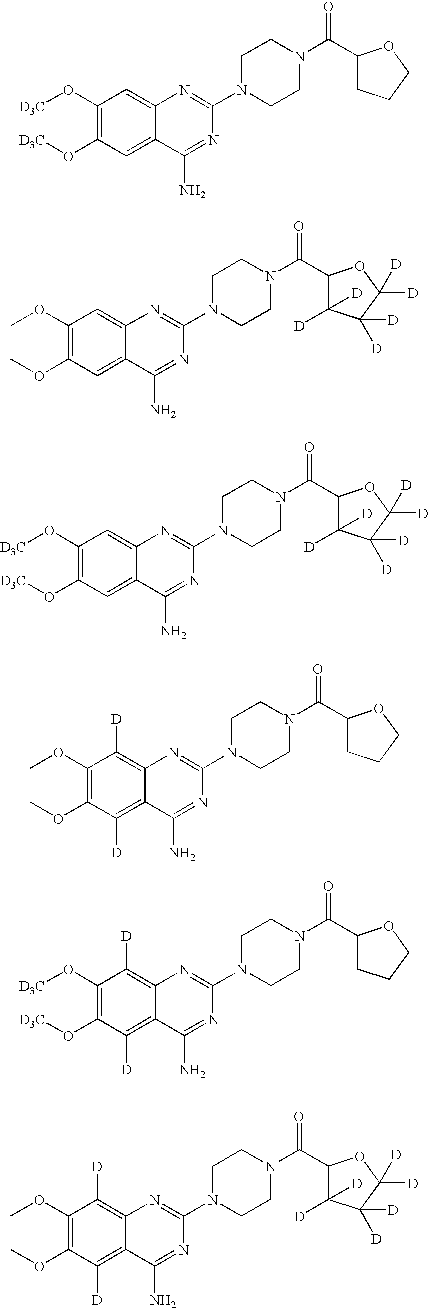 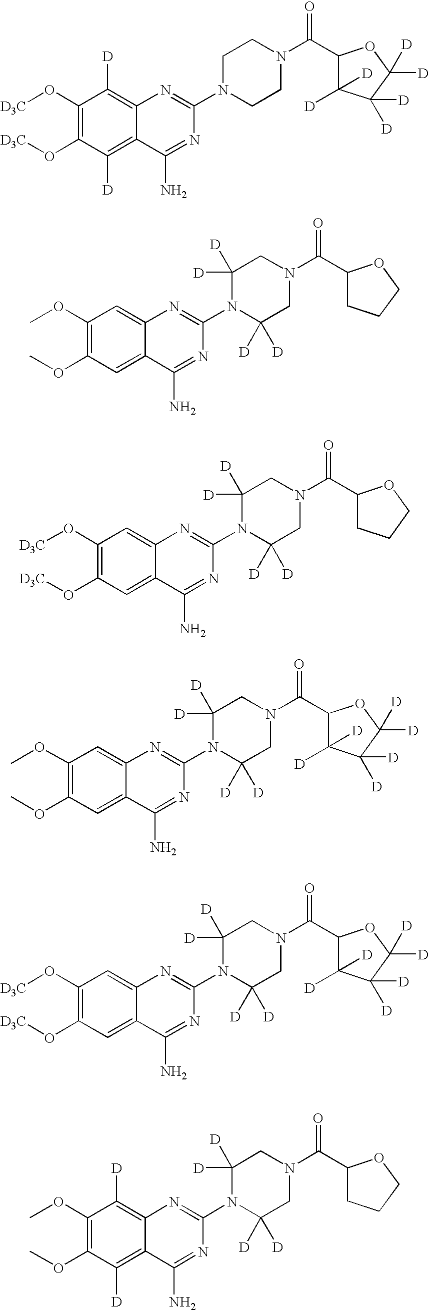 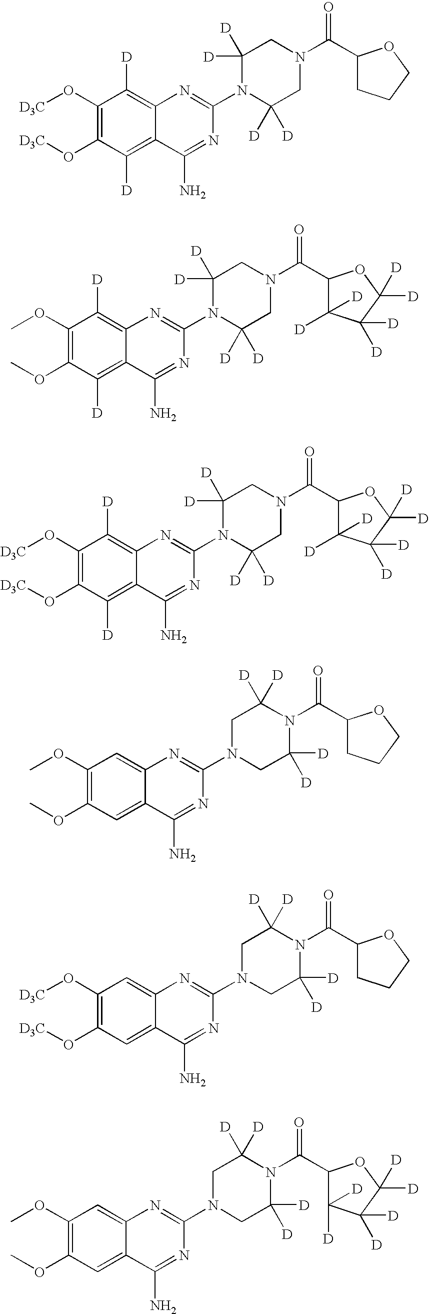 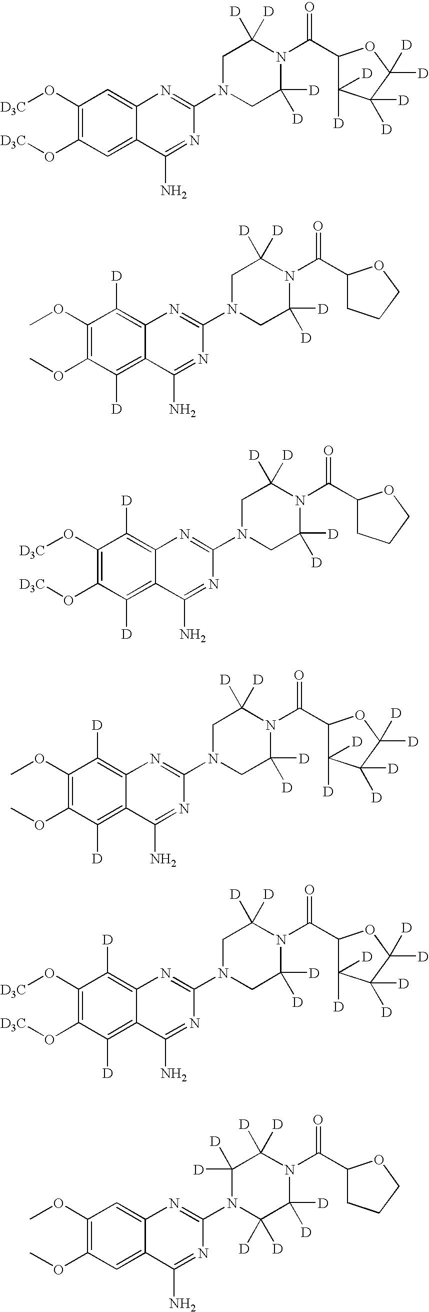 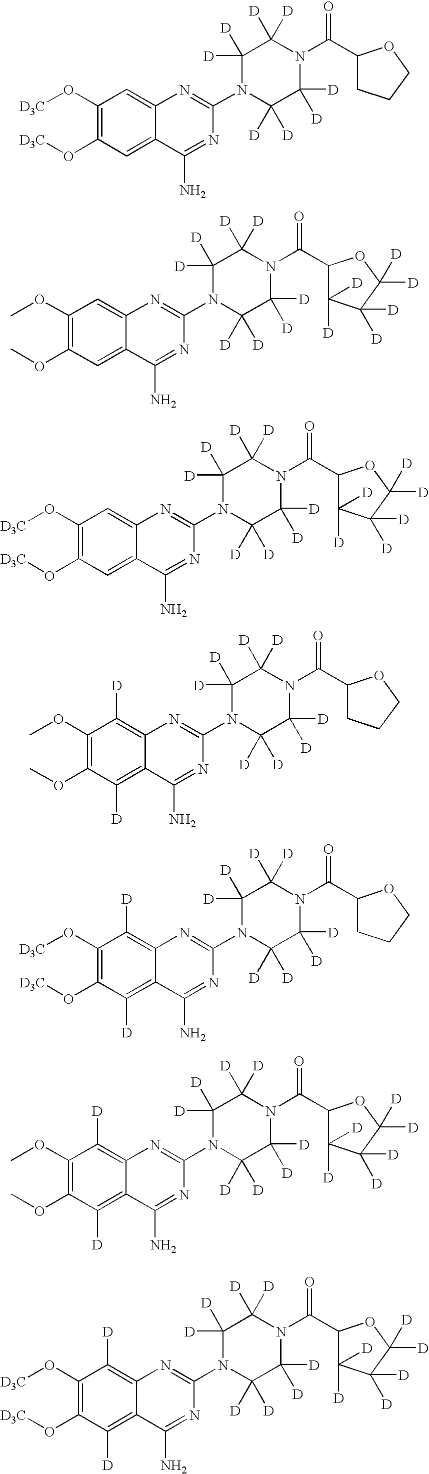 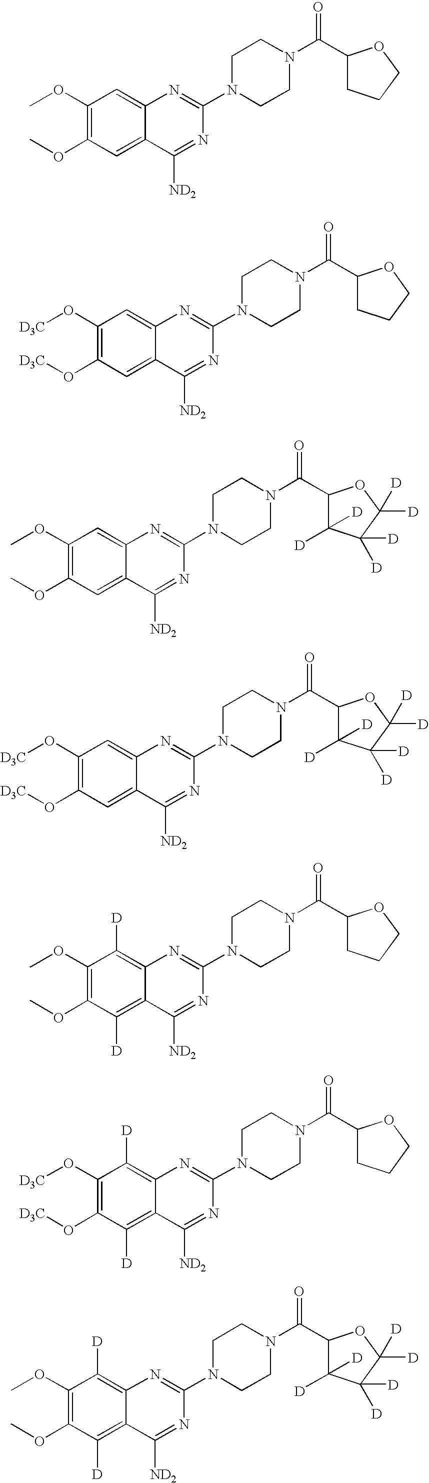 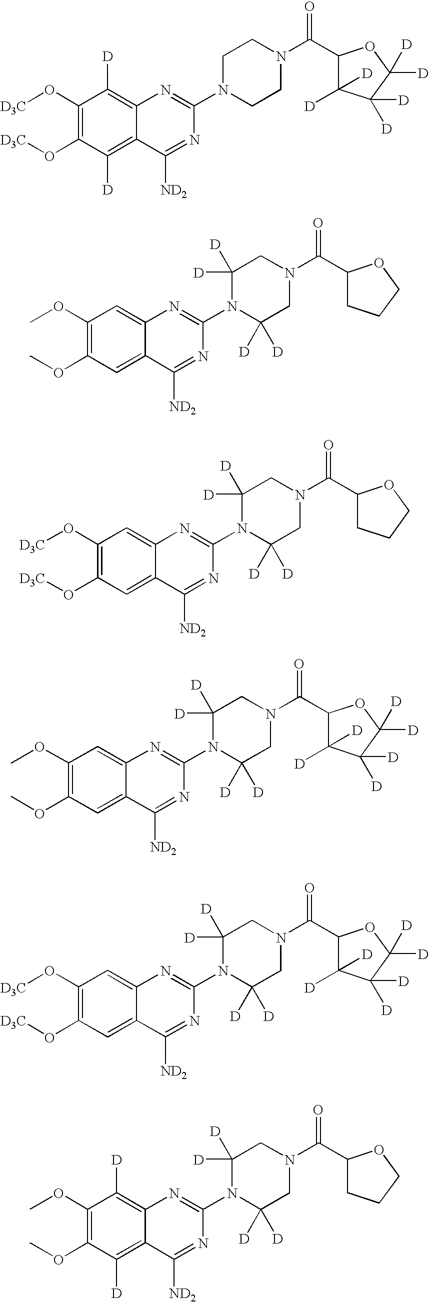 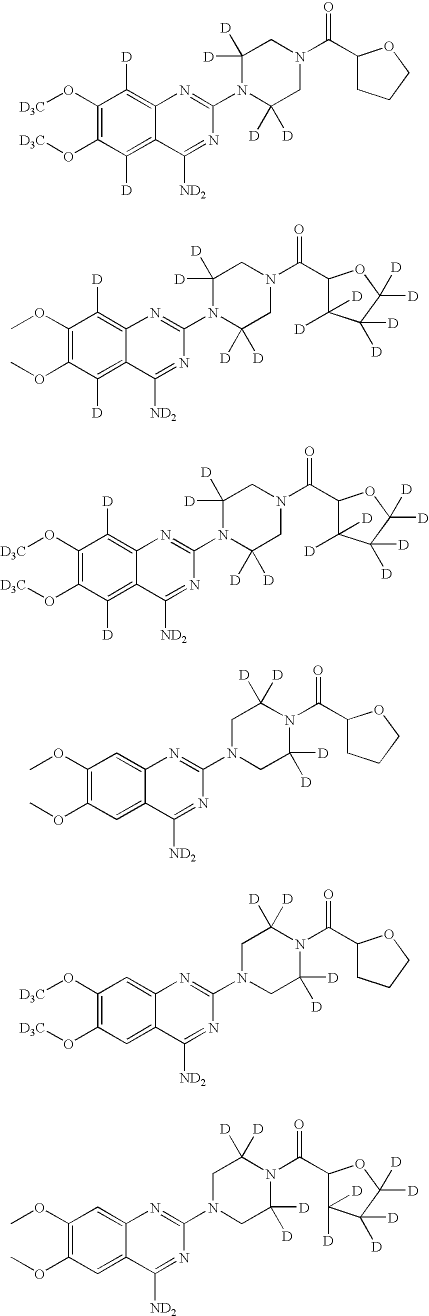 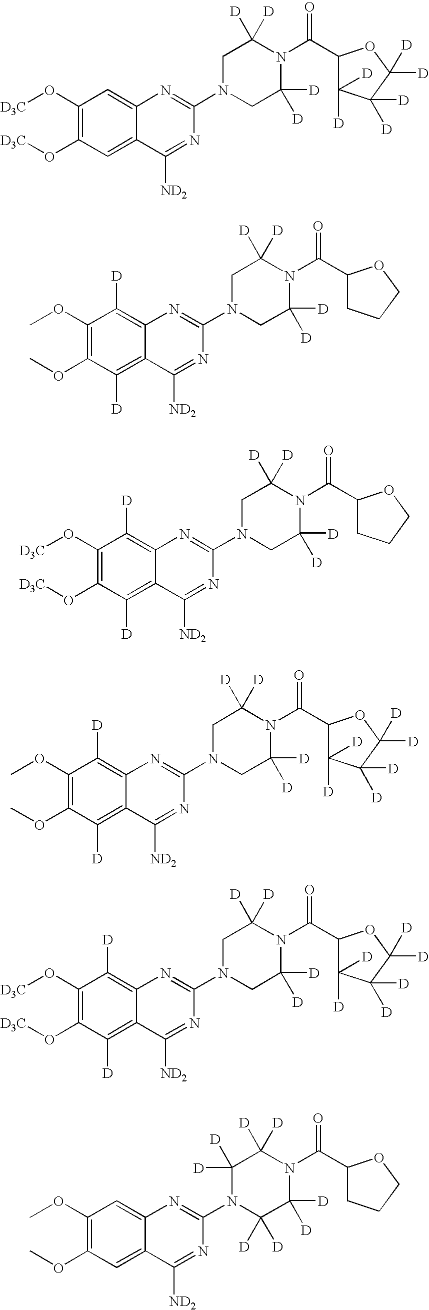 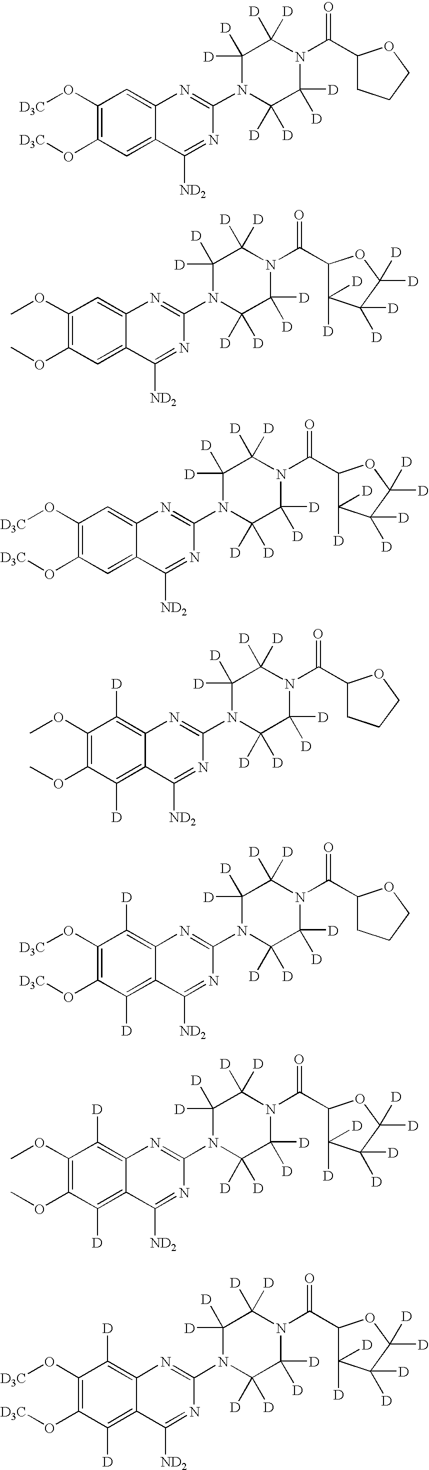 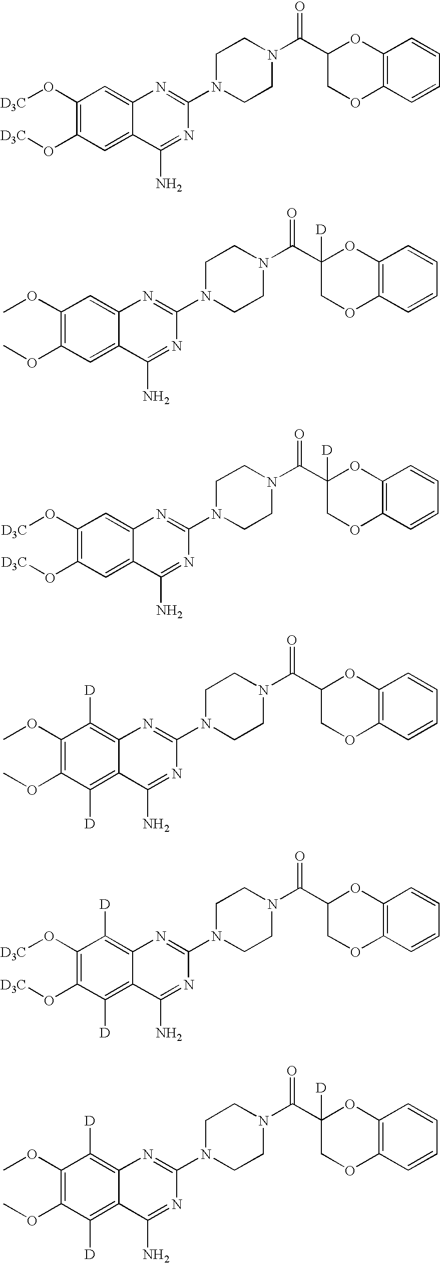 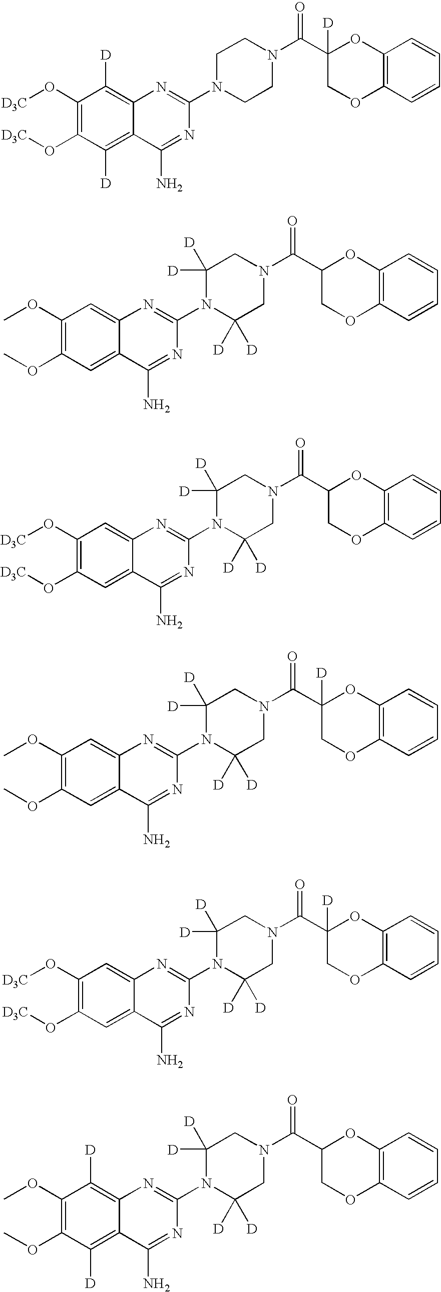 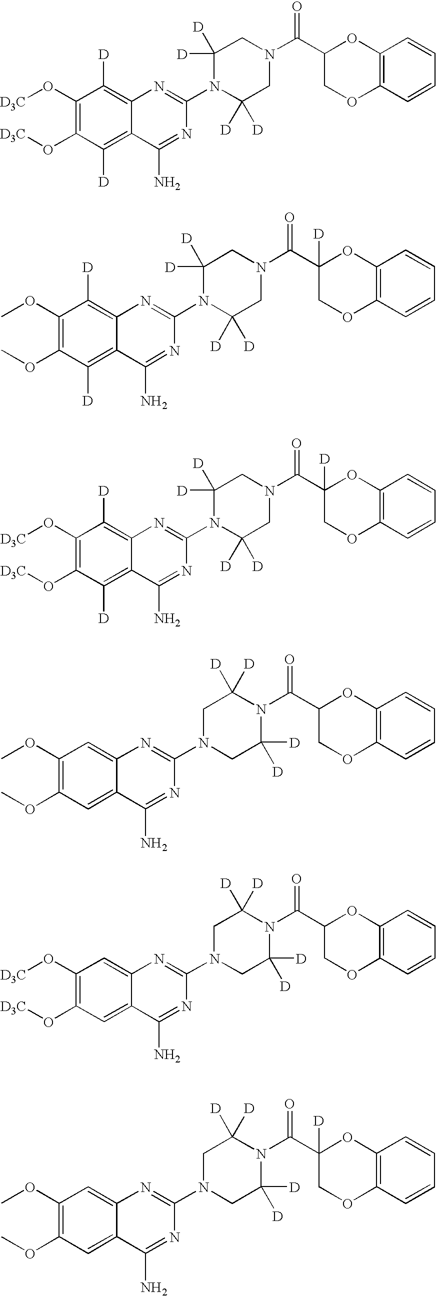 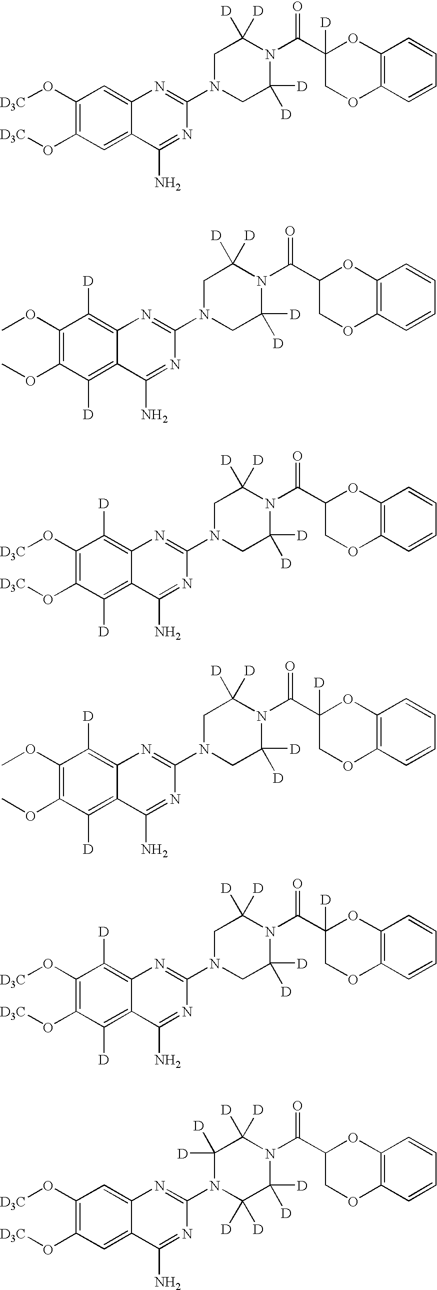 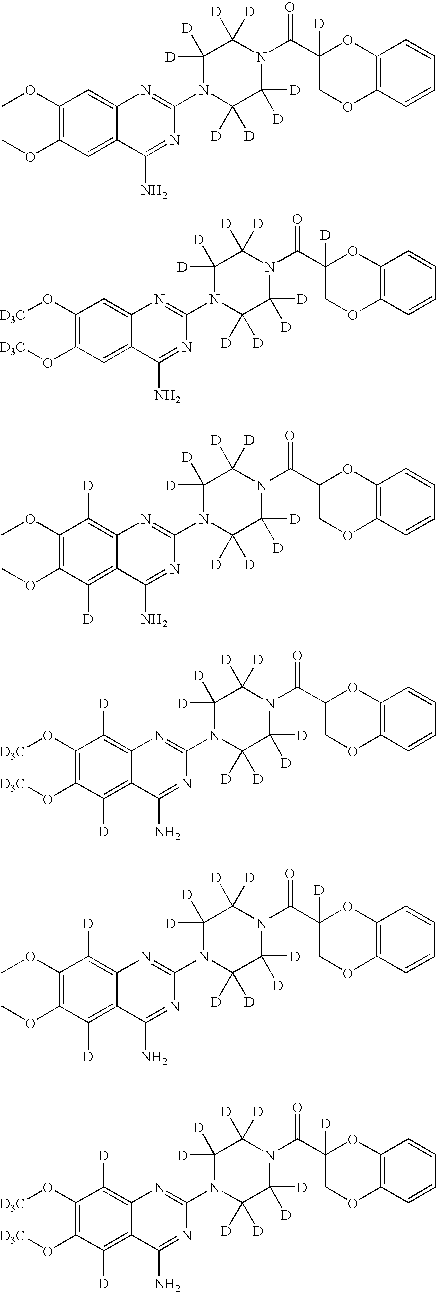 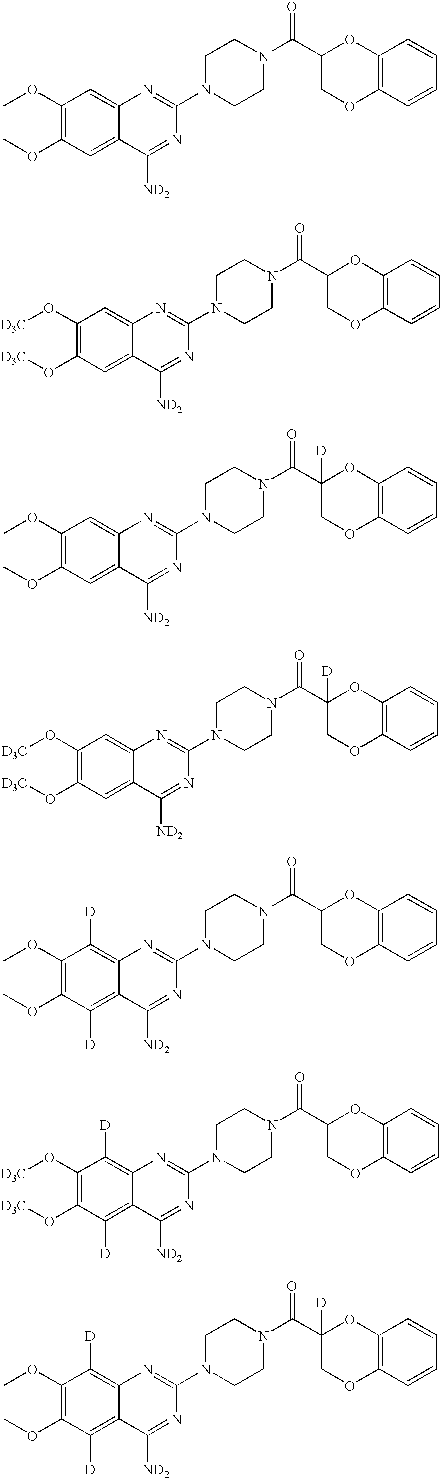 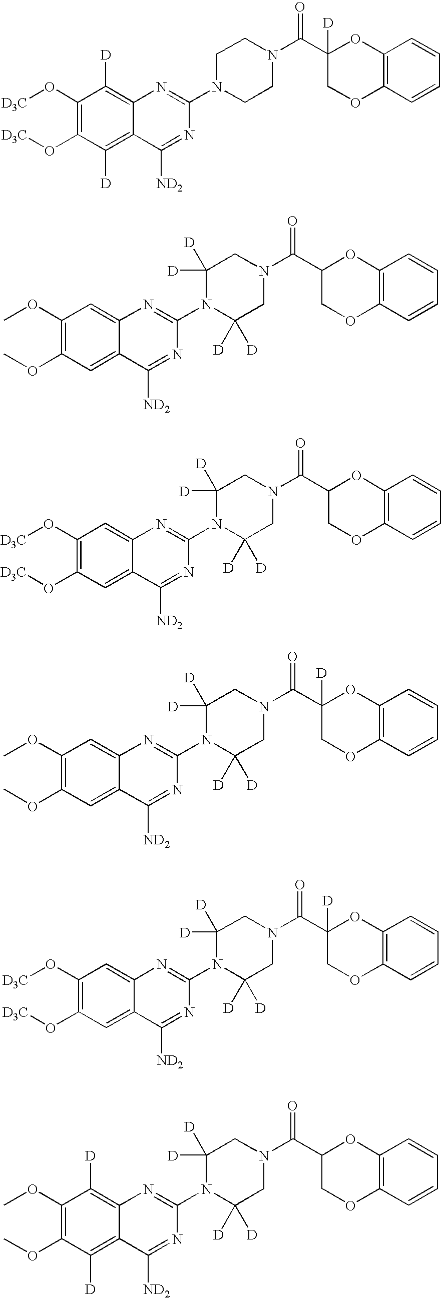 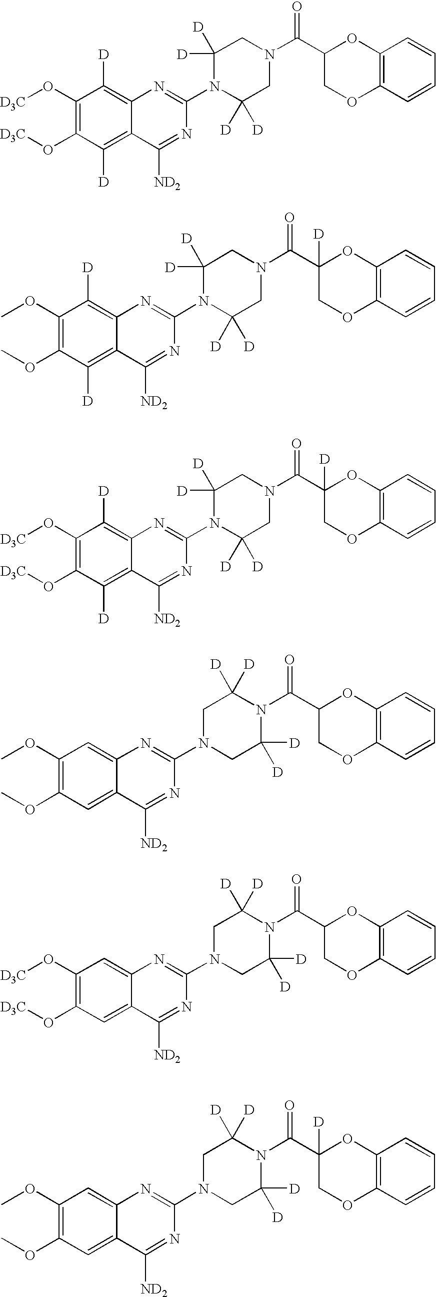 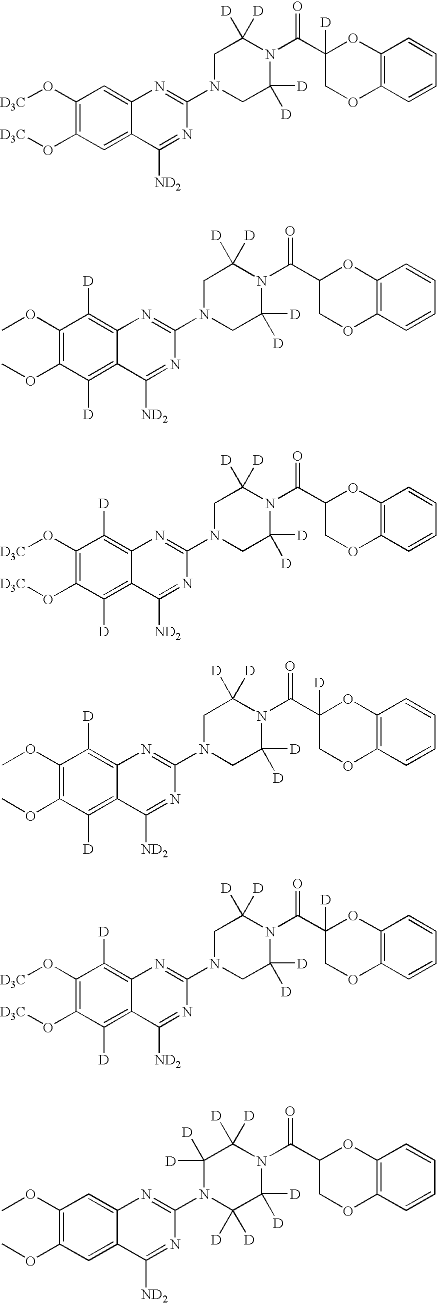 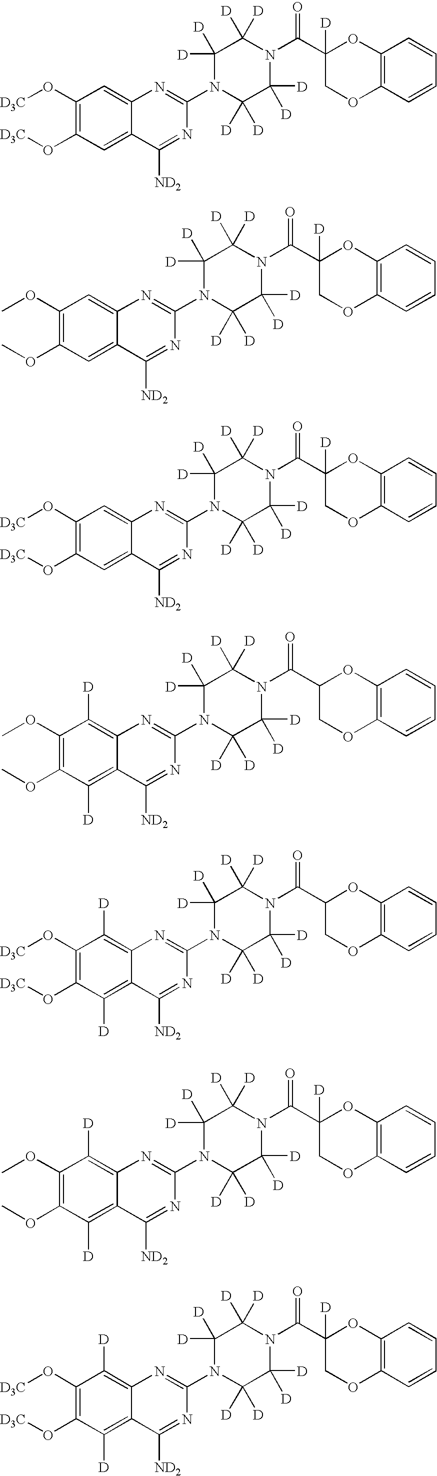 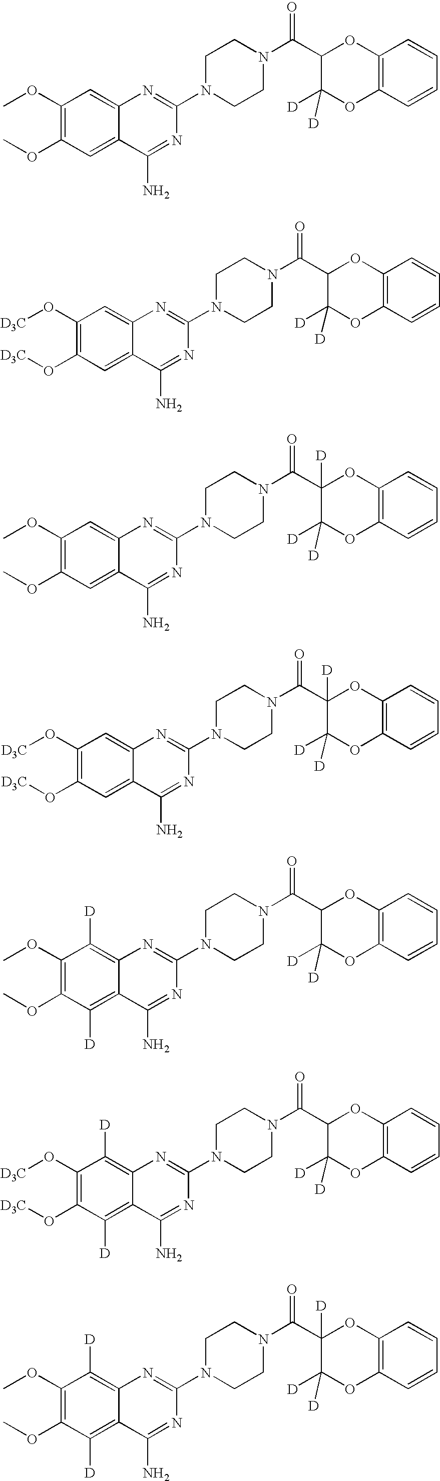 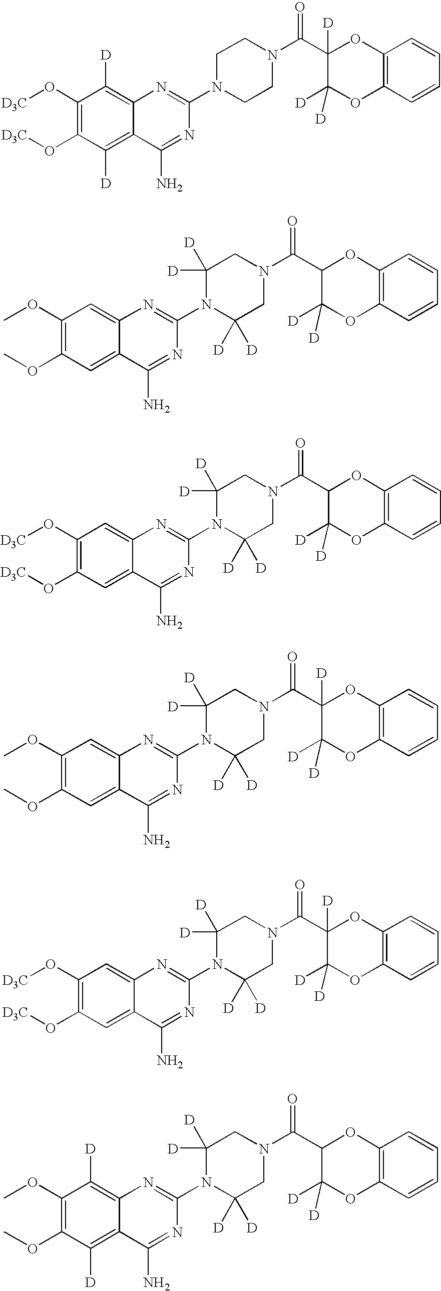 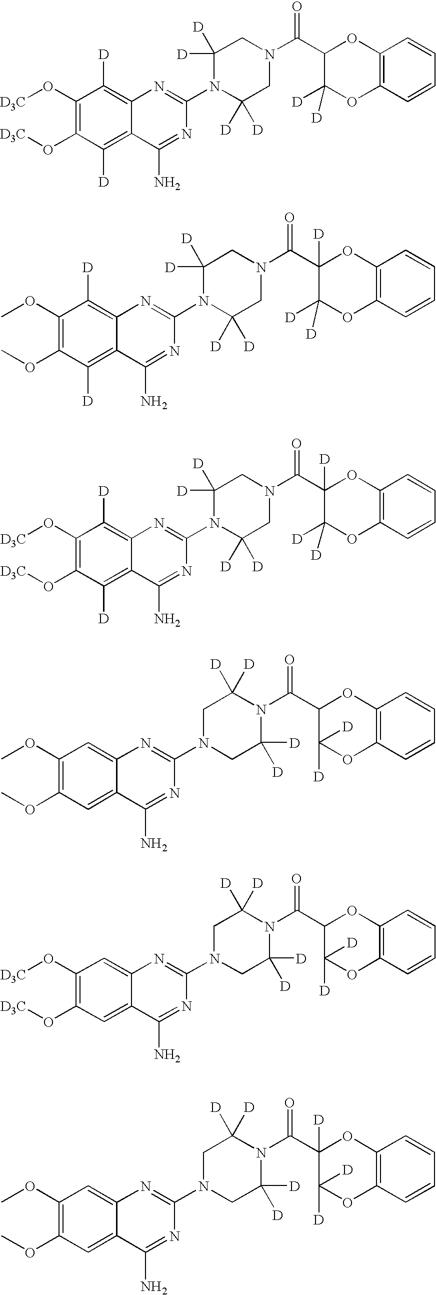 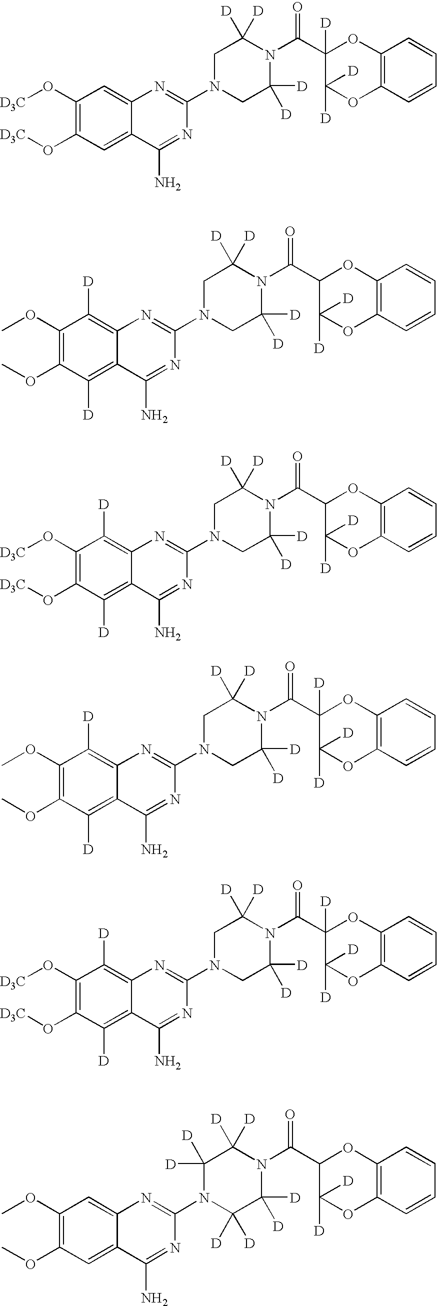 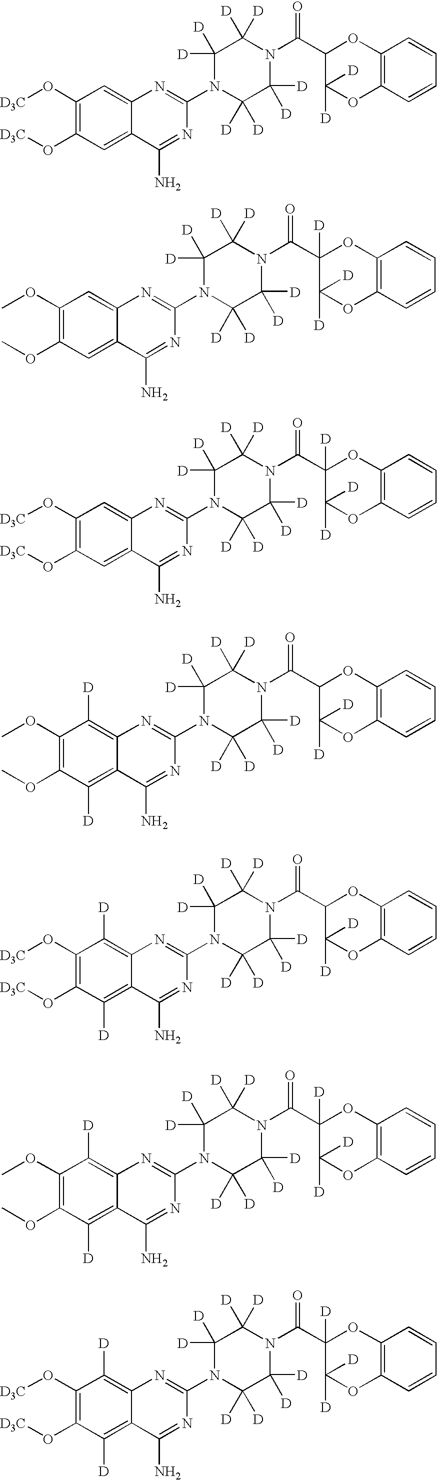 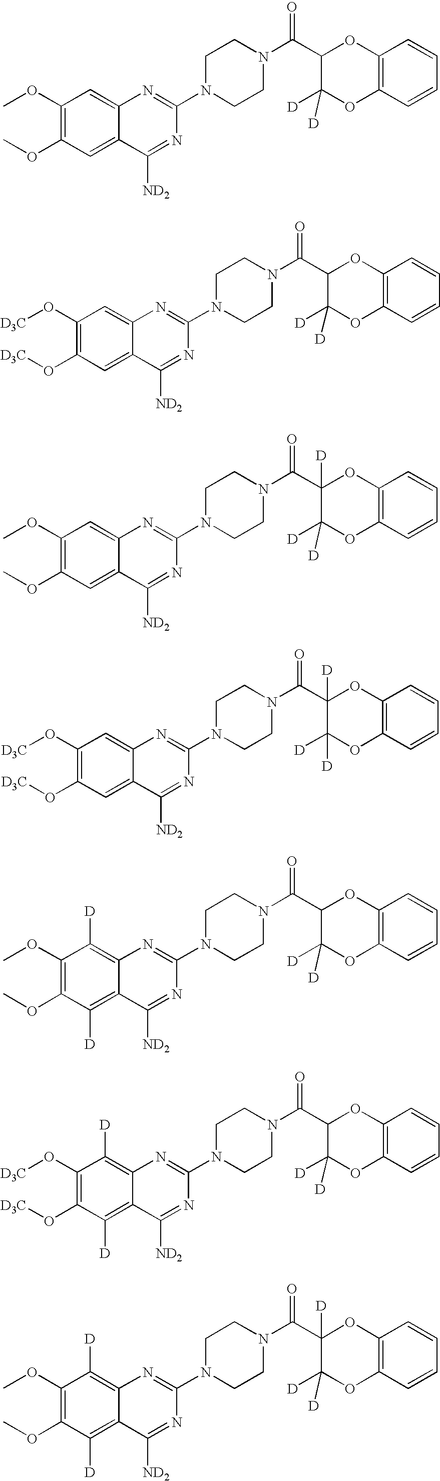 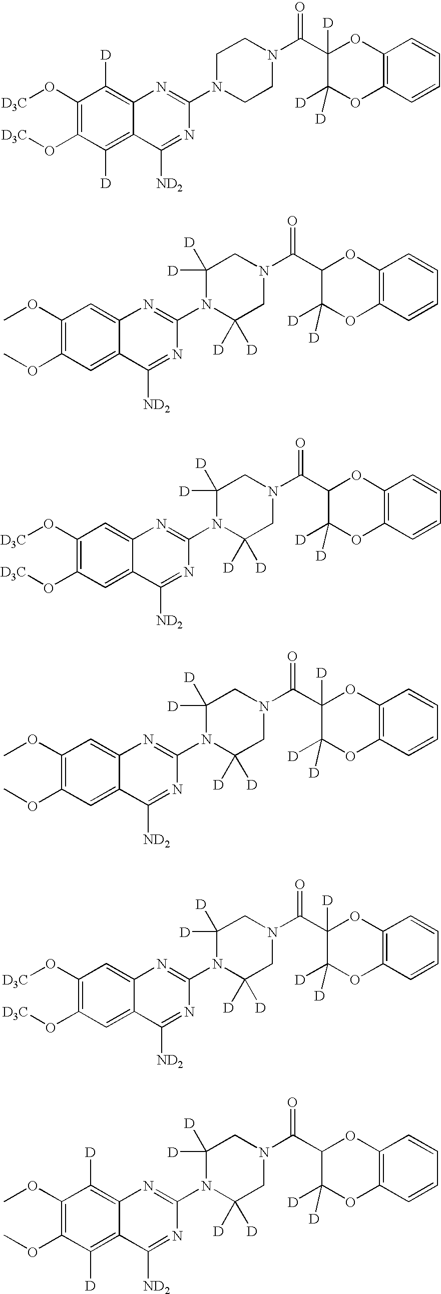 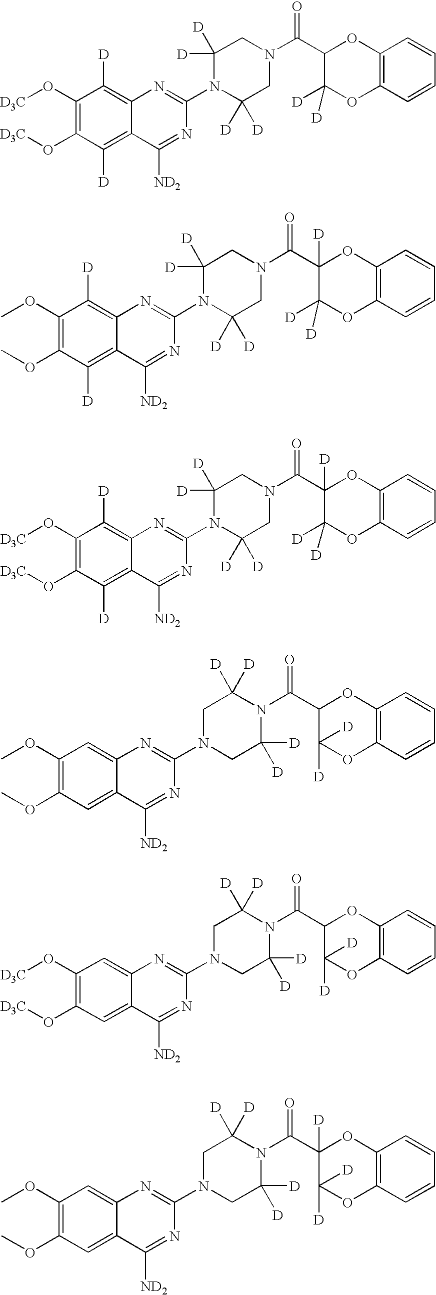 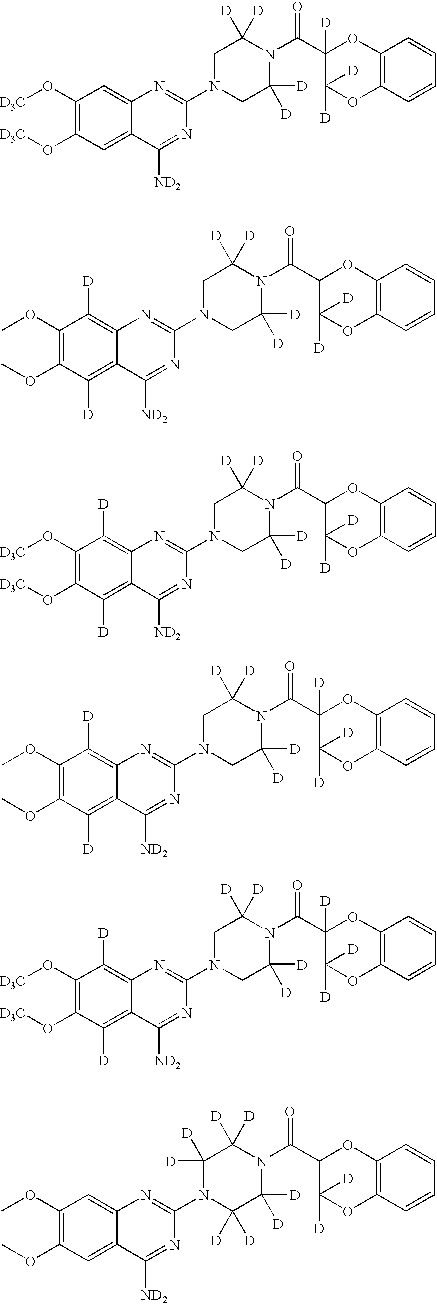 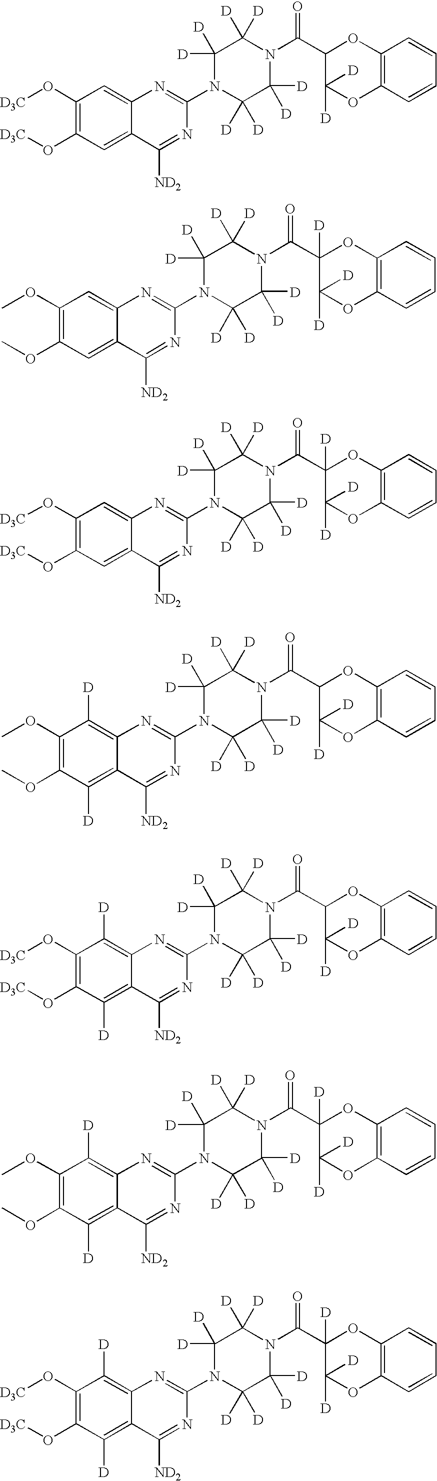 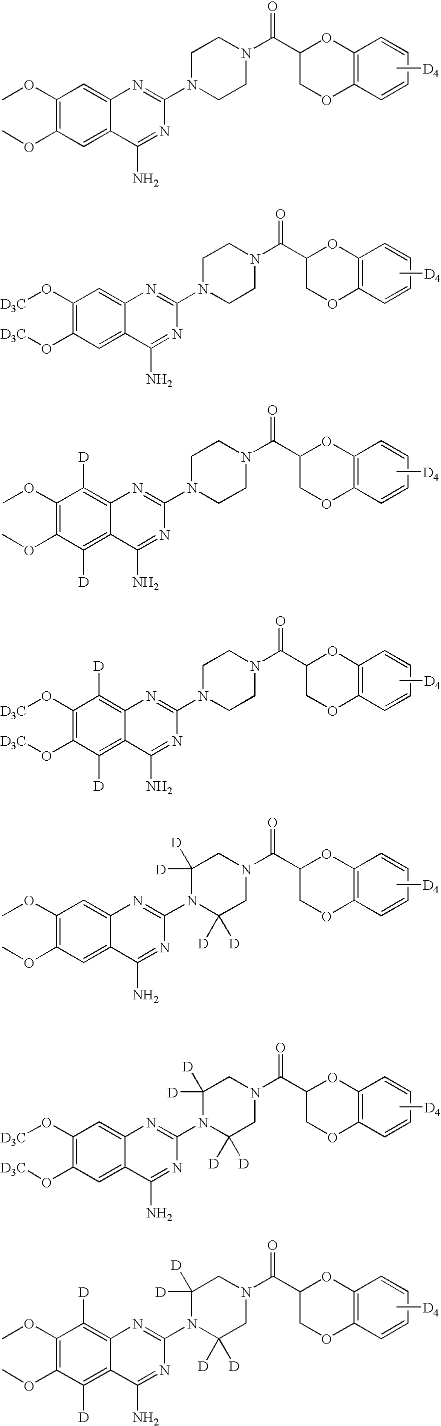 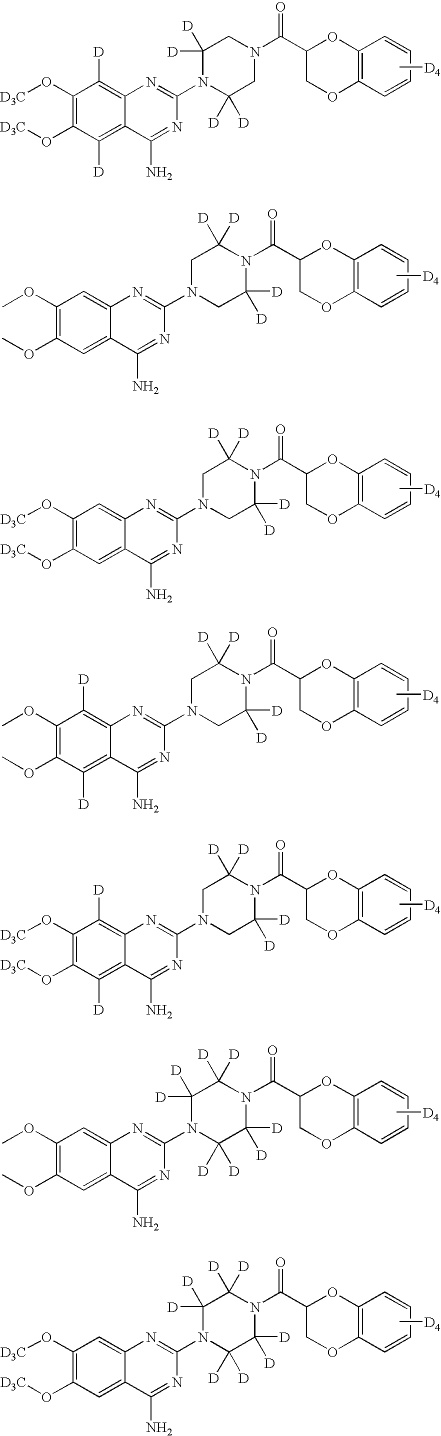 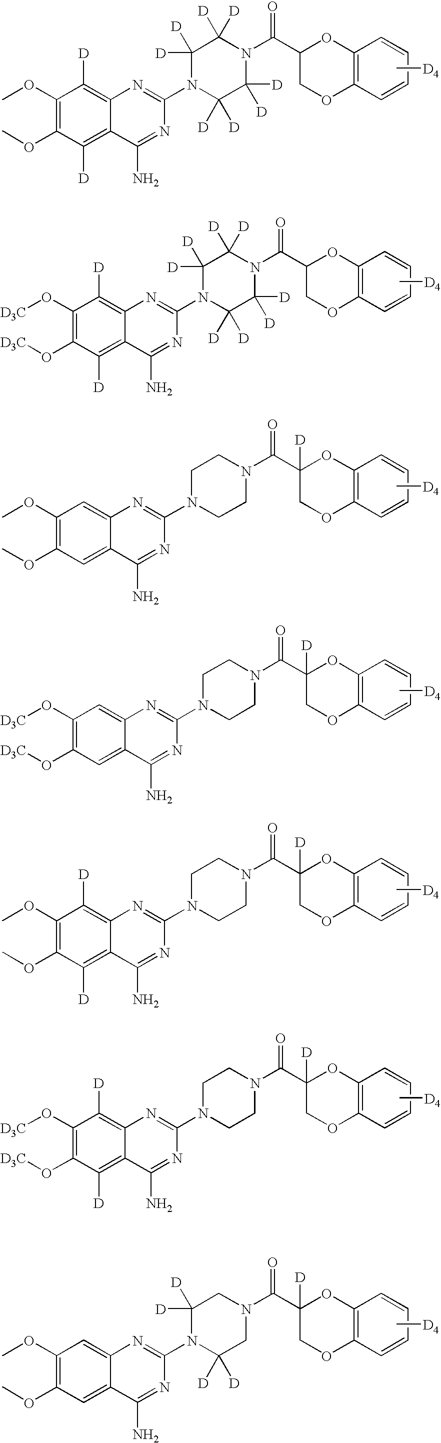 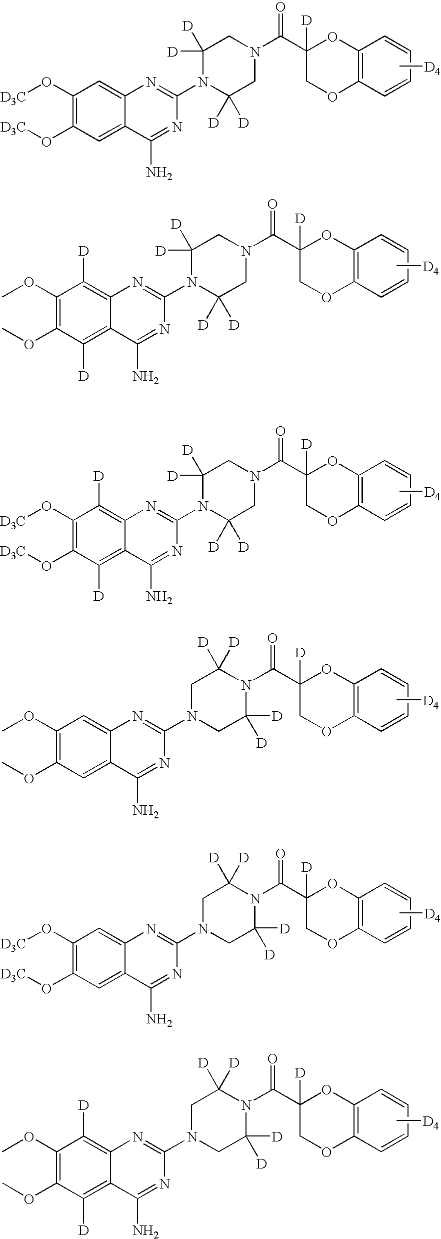 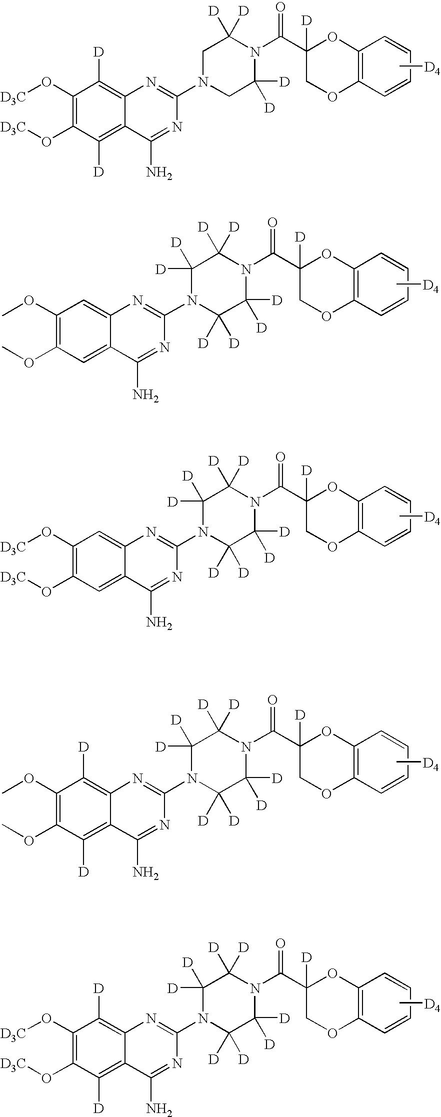 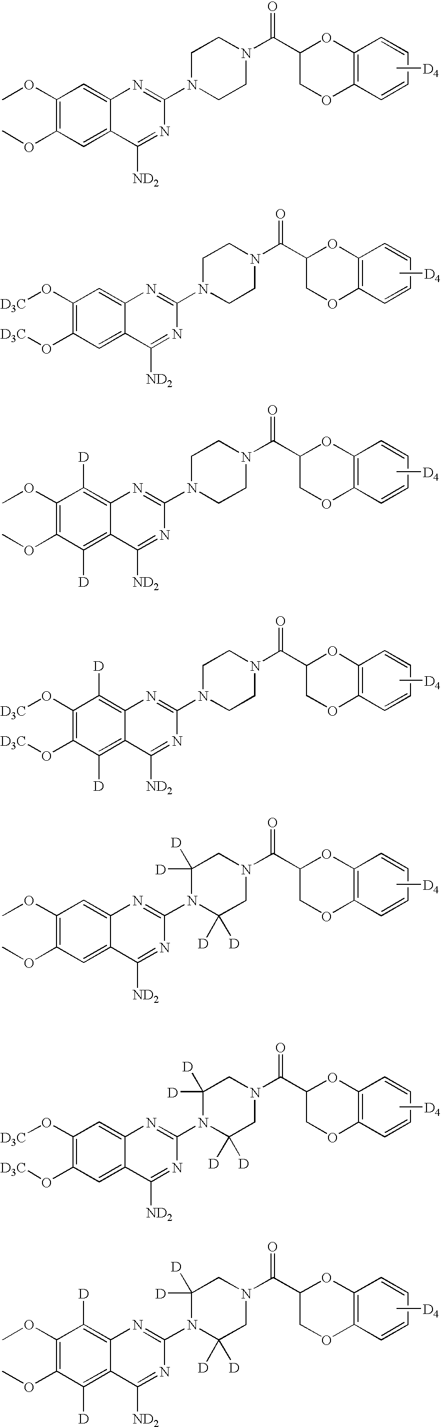 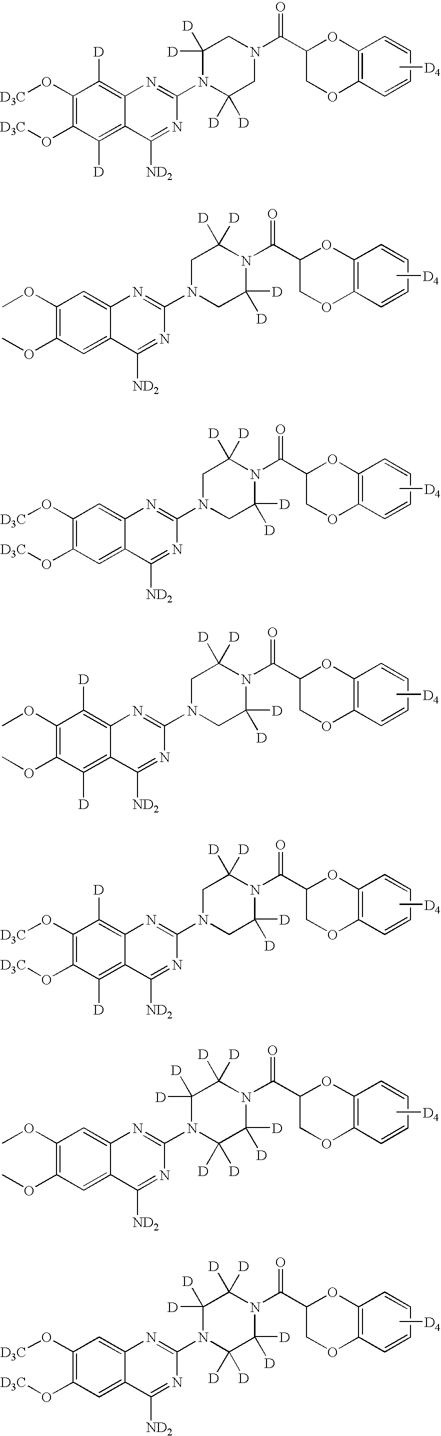 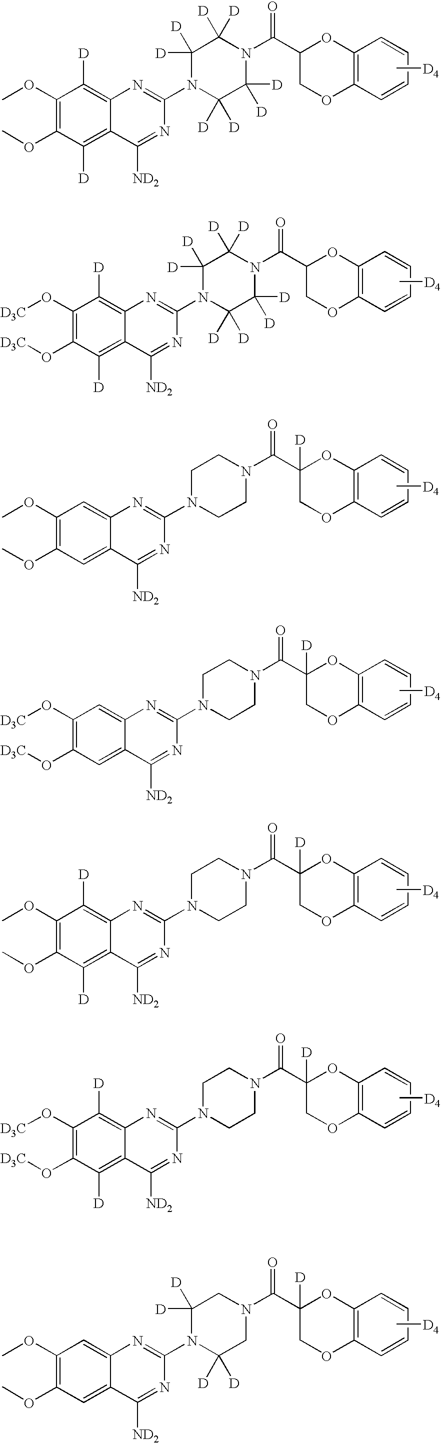 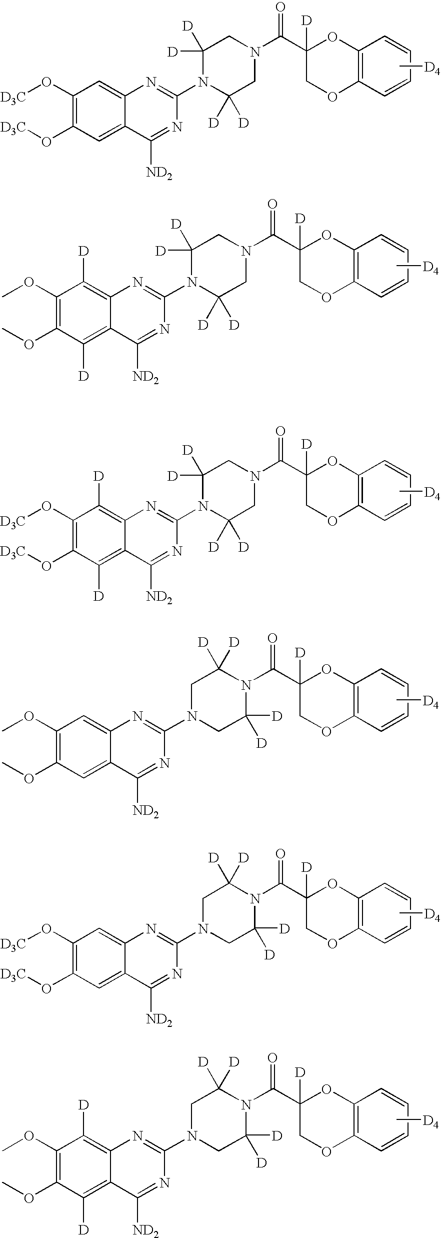 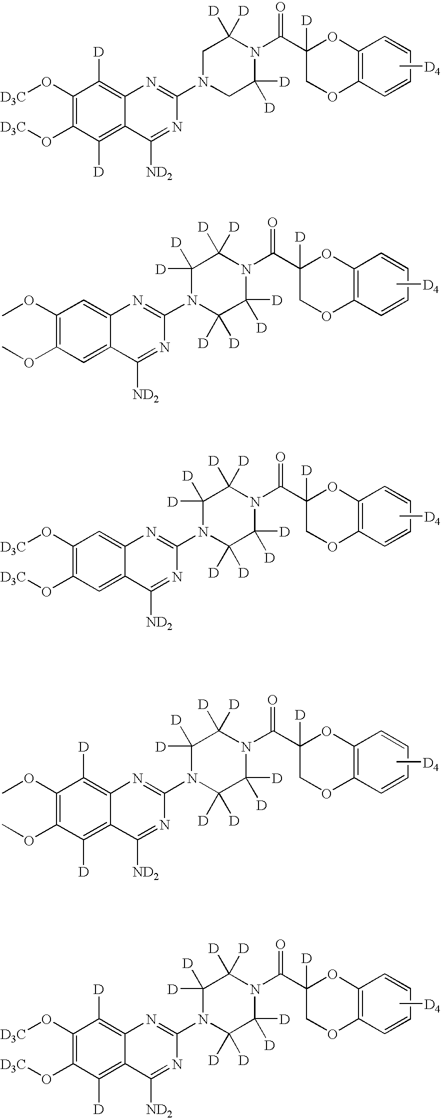 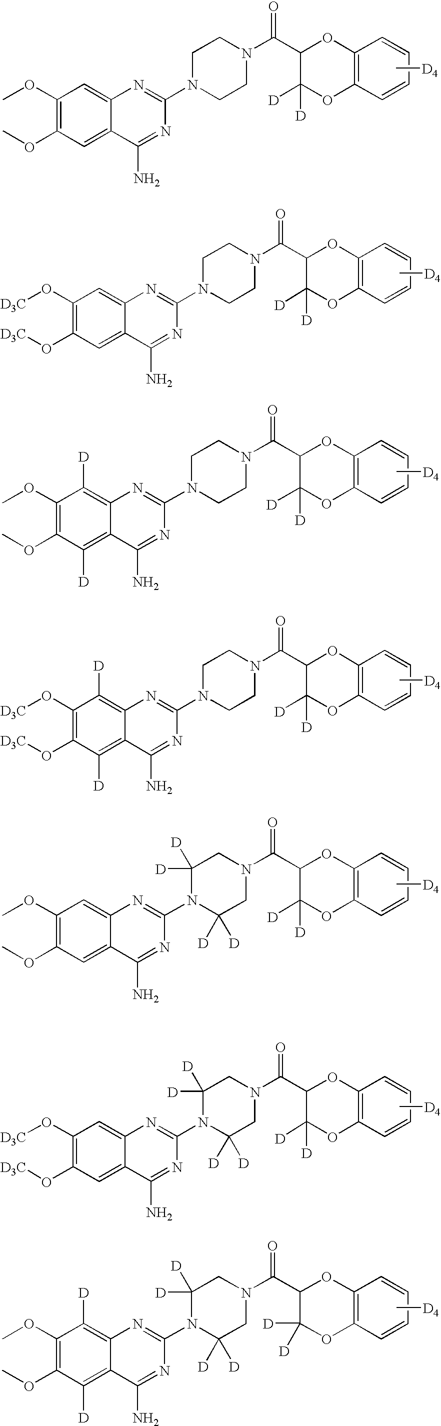 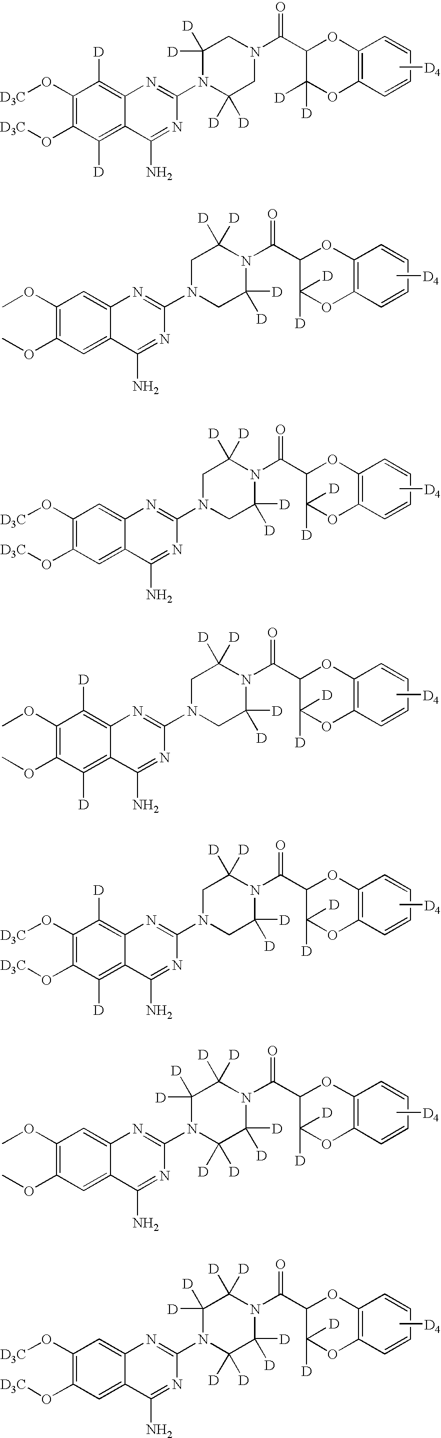 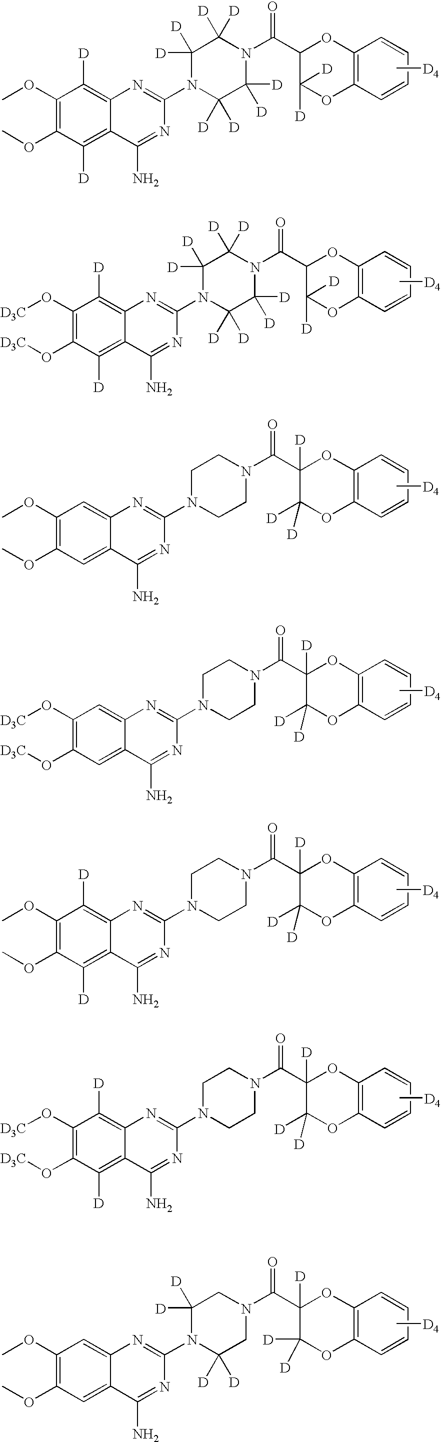 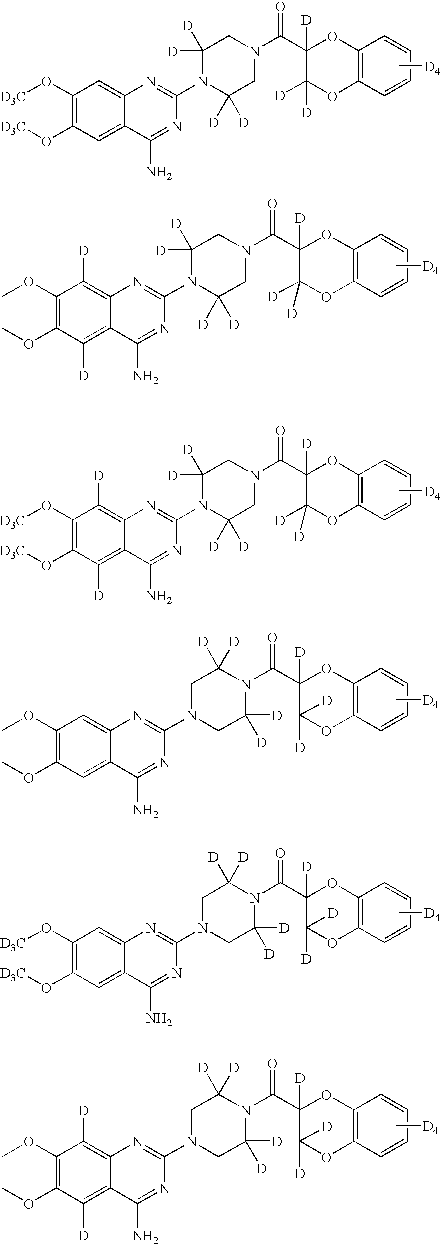 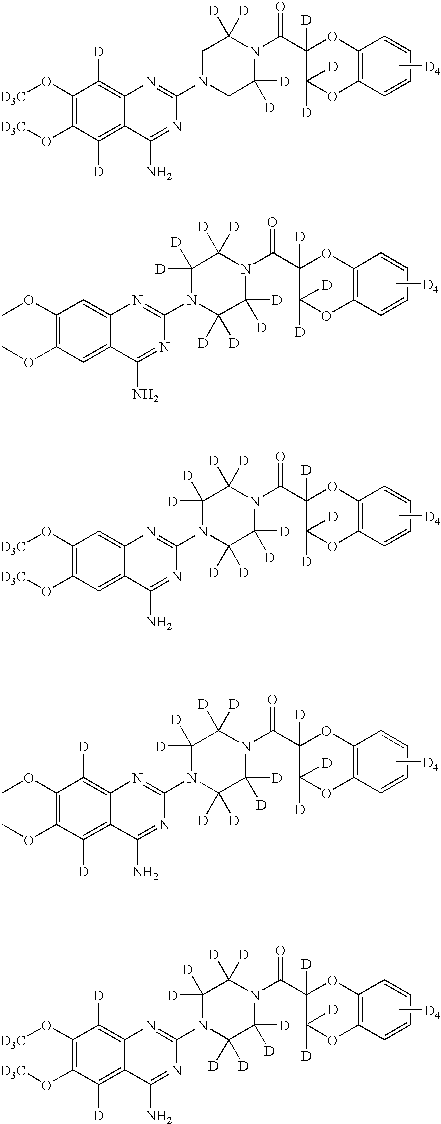 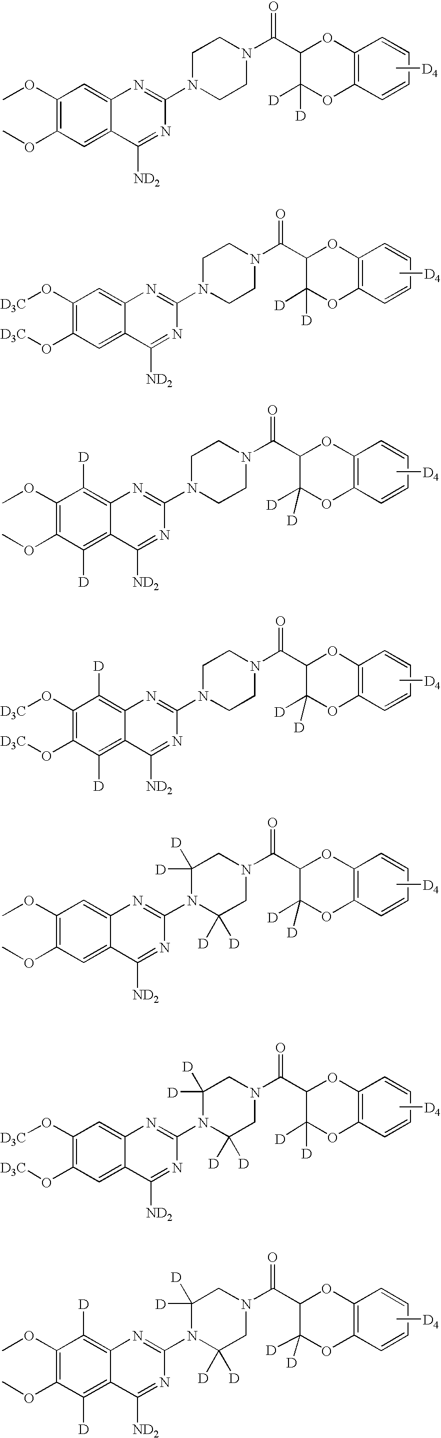 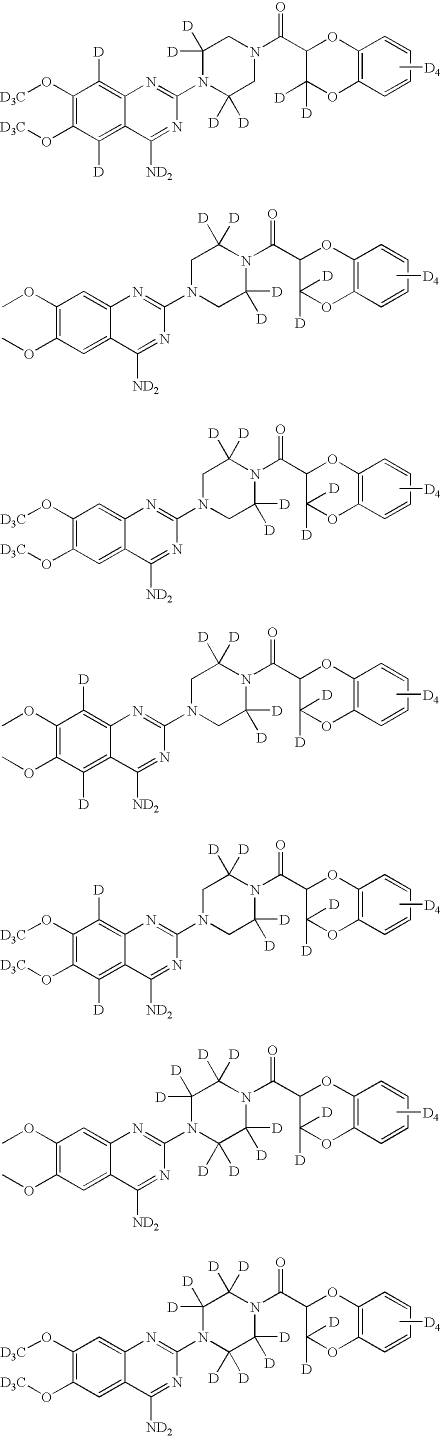 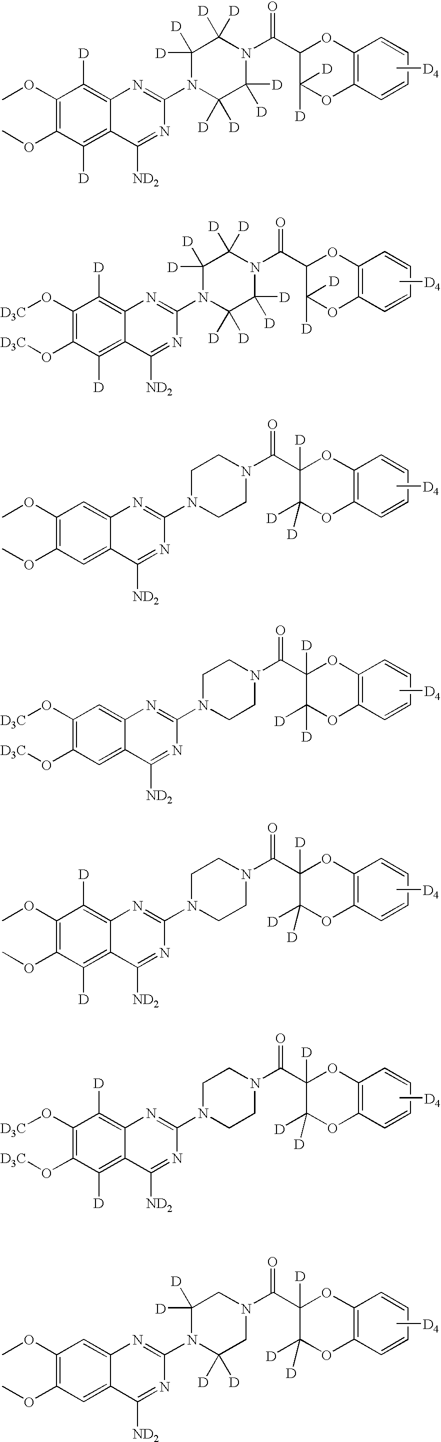 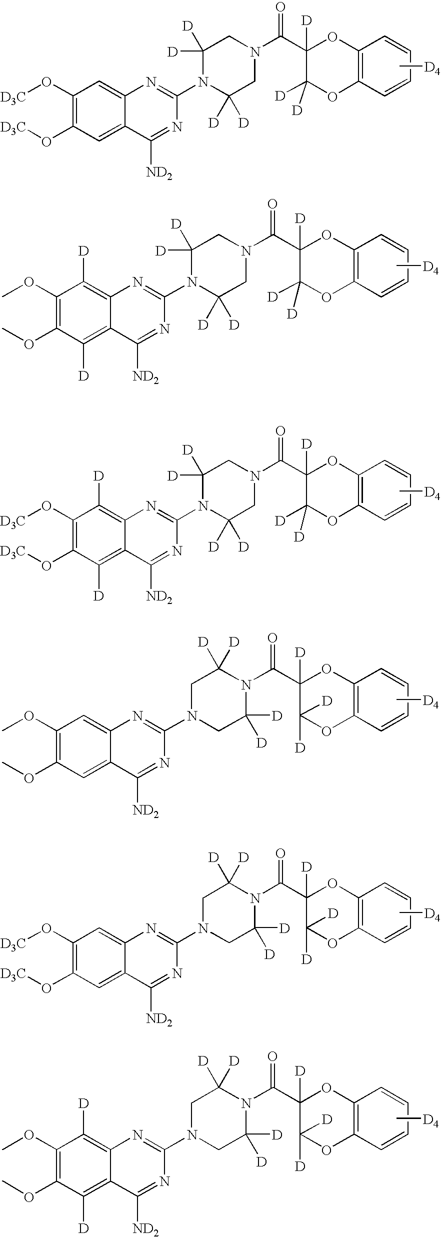 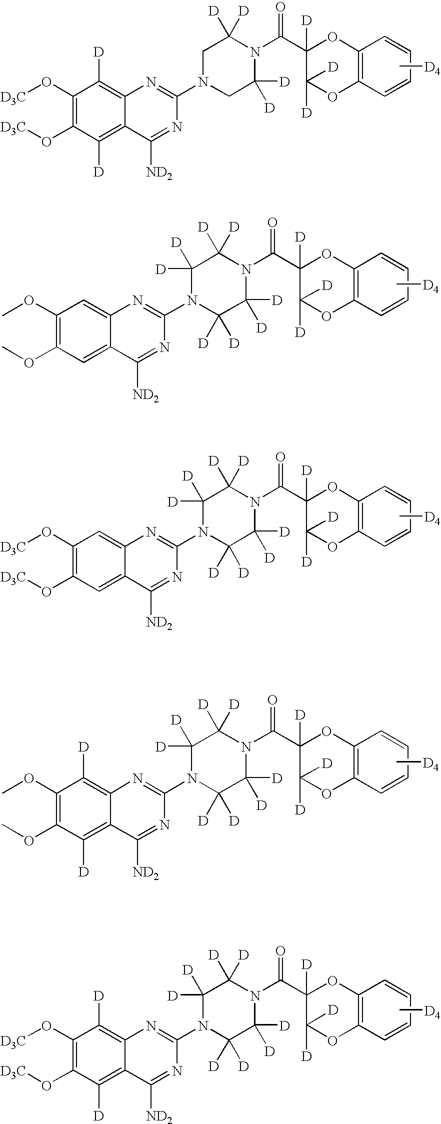 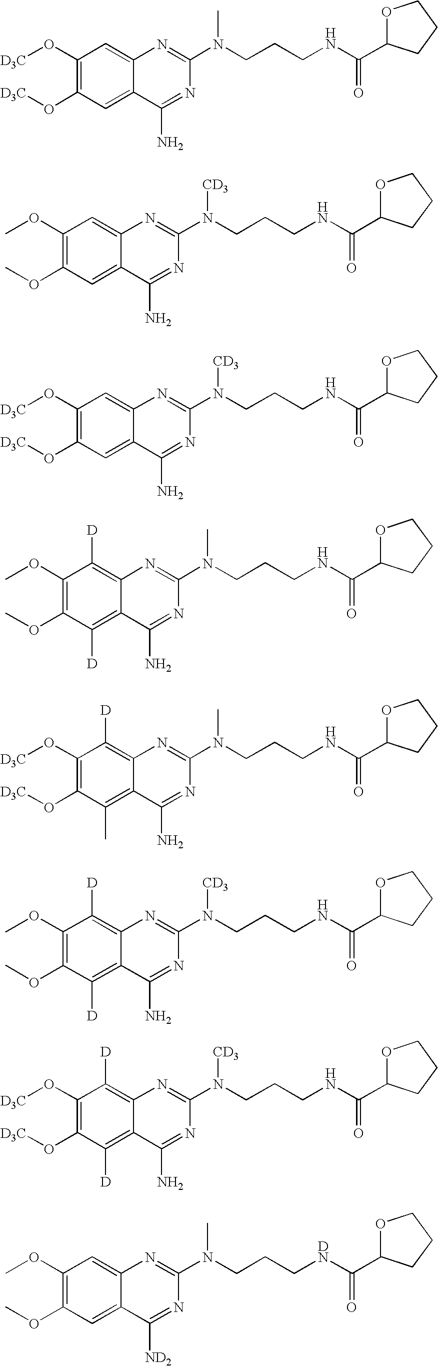 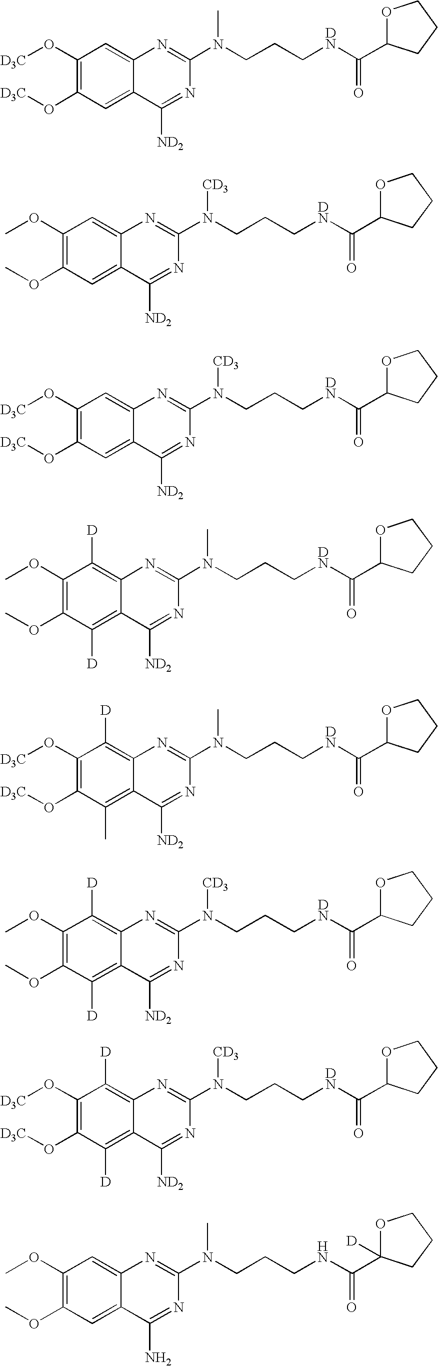 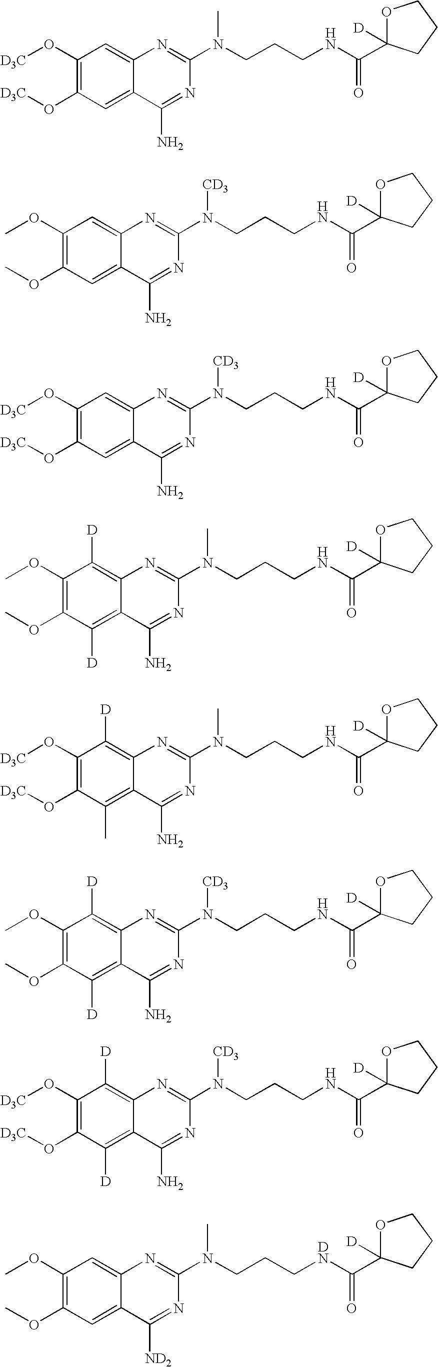 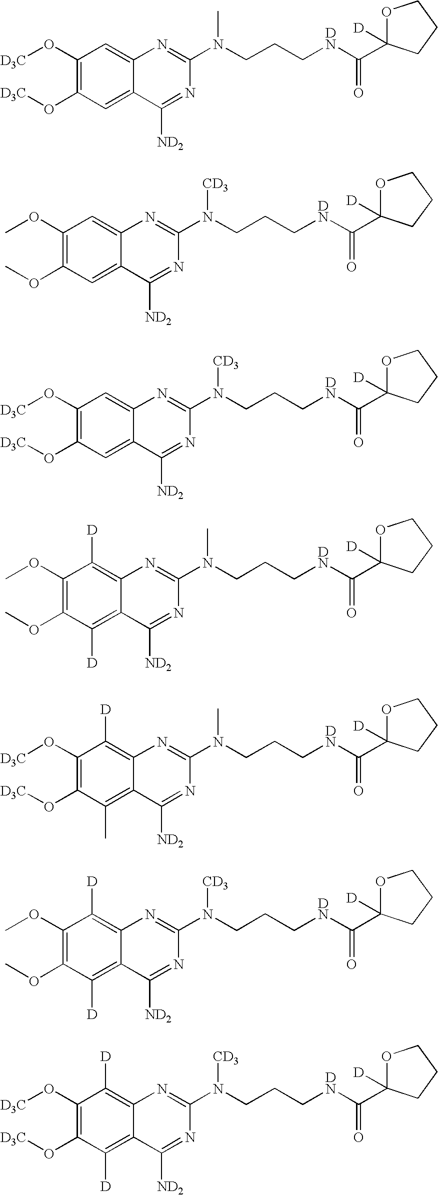 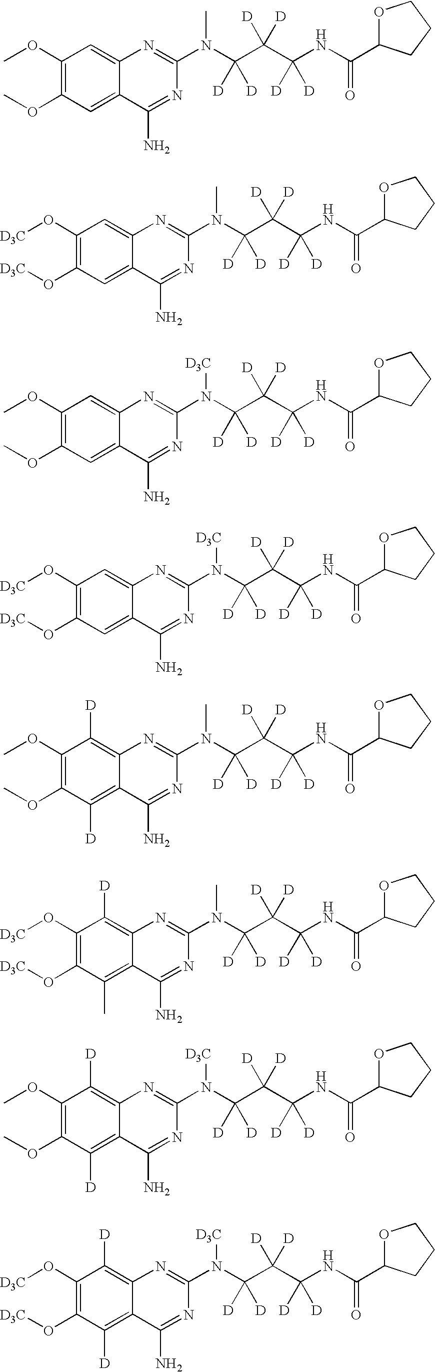 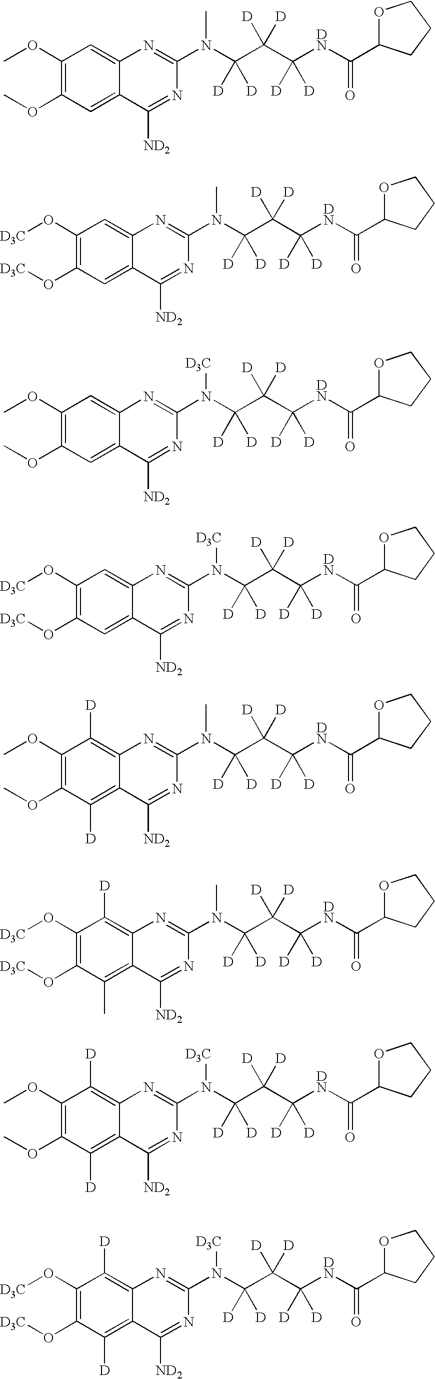 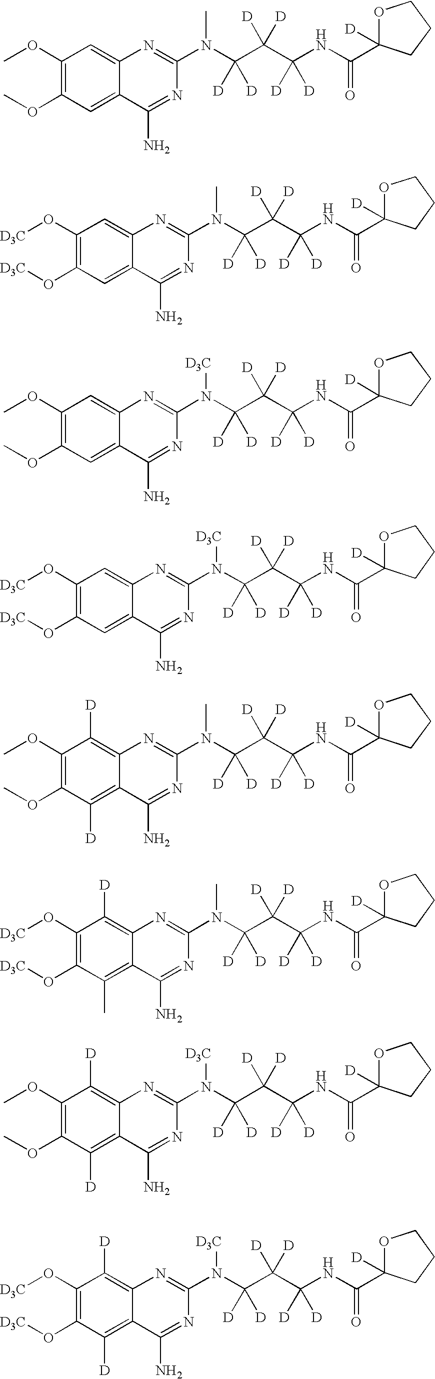 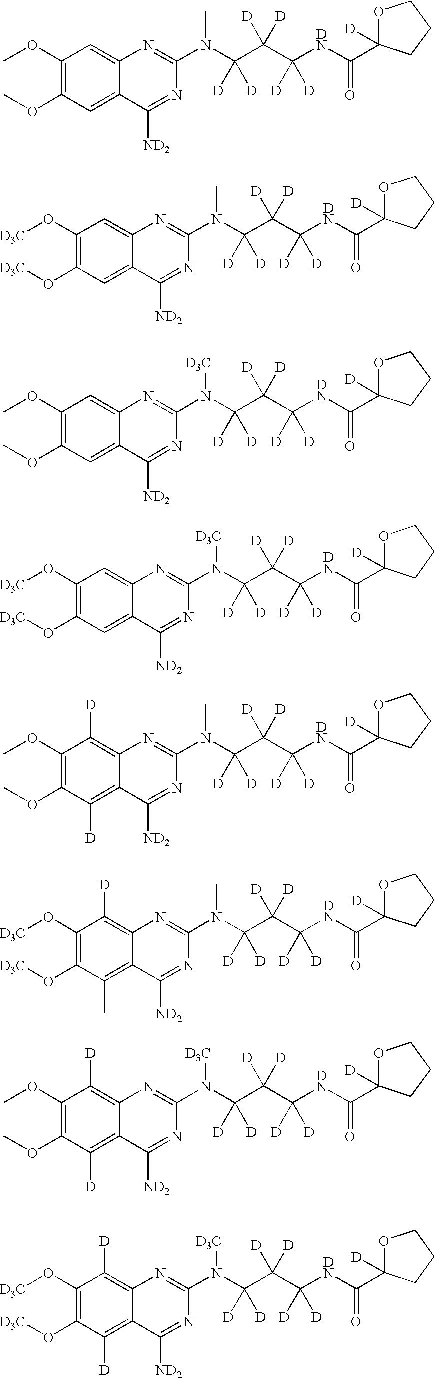
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof.

5. A method of treating a mammal suffering from a disease or condition in which it is beneficial to modulate an alpha-adrenergic receptor, comprising administering to said mammal a therapeutically effective amount of a compound of Formula 1 so as to affect decreased inter-individual variation in plasma levels of said compound or a metabolite thereof as compared to the non-isotopically enriched compound;
wherein said compound of Formula 1 has the structure: 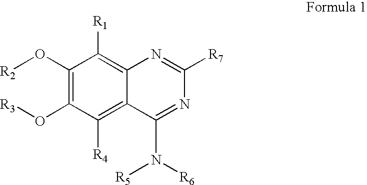
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 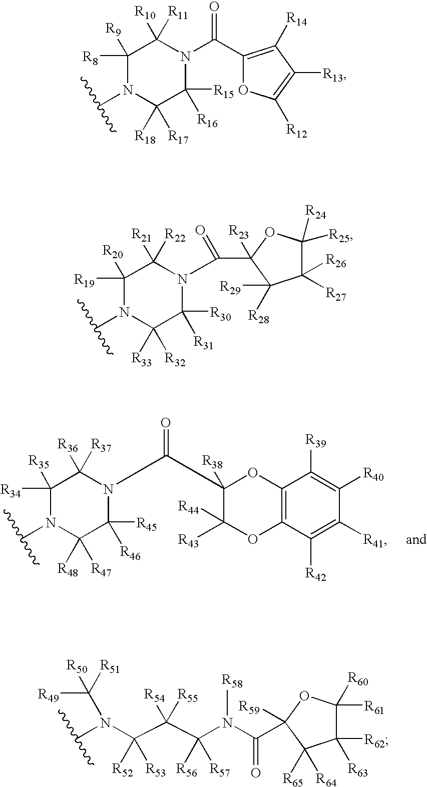
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%
with the proviso that compounds of Formula 1 cannot be selected from the group consisting of:

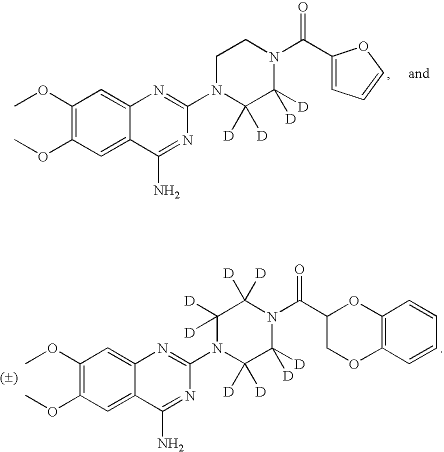

6. A method of treating a mammal suffering from a disease or condition in which it is beneficial to modulate an alpha-adrenergic receptor, comprising administering to said mammal a therapeutically effective amount of a compound of Formula 1 so as to affect increased average plasma levels of said compound per dosage unit thereof as compared to the non-isotopically enriched compound;
wherein said compound of Formula 1 has the structure: 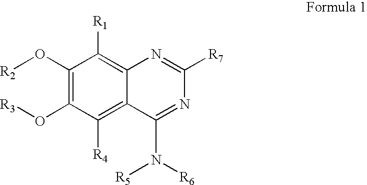
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 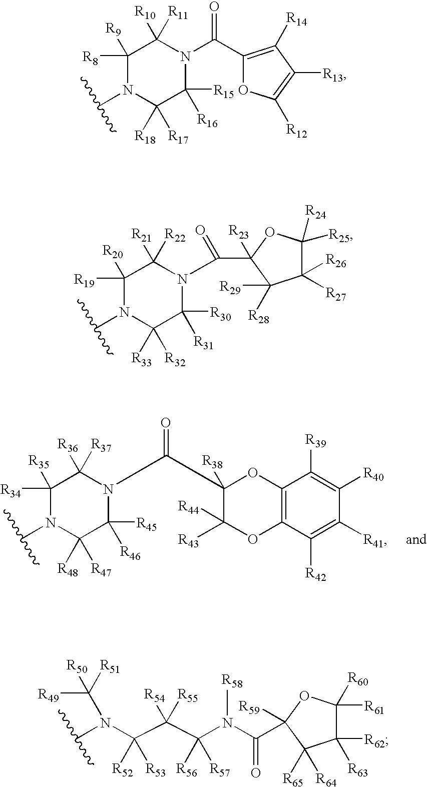
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%
with the proviso that compounds of Formula 1 cannot be selected from the group consisting of:

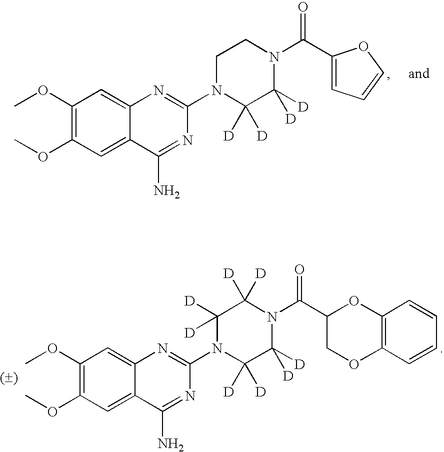

7. A method of treating a mammal suffering from a disease or condition in which it is beneficial to modulate an alpha-adrenergic receptor, comprising administering a therapeutically effective amount of a compound of Formula 1 so as to affect decreased average plasma levels of at least one metabolite of said compound per dosage unit thereof as compared to the non-isotopically enriched compound,
wherein said compound of Formula 1 has the structure: 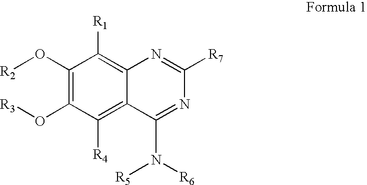
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers, or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 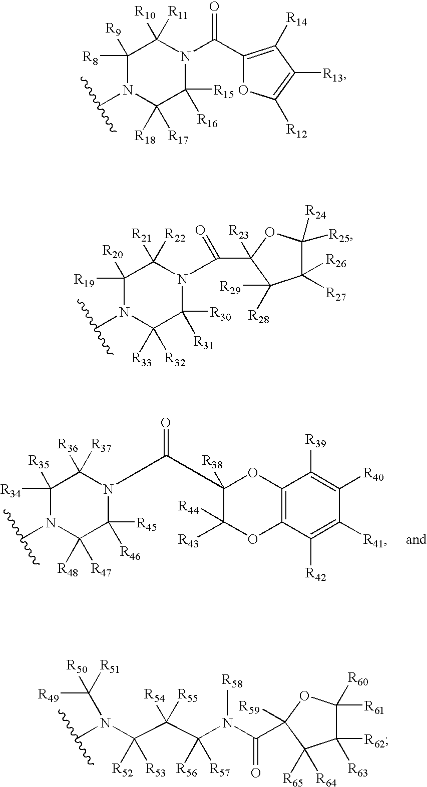
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%
with the proviso that compounds of Formula 1 cannot be selected from the group consisting of:

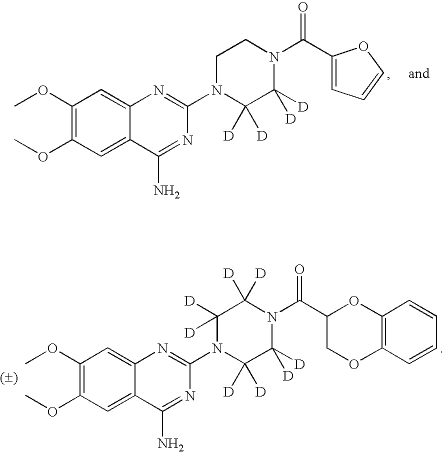

8. A method of treating a mammal suffering from a disease or condition in which it is beneficial to modulate an alpha-adrenergic receptor, comprising administering a therapeutically effective amount of a compound of Formula 1 so as to affect a decreased metabolism by at least one polymorphically-expressed cytochrome P₄₅₀ isoform in mammalian subjects per dosage unit thereof as compared to the non-isotopically enriched compound, wherein said compound of Formula 1 has the structure: 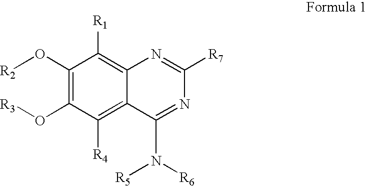
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 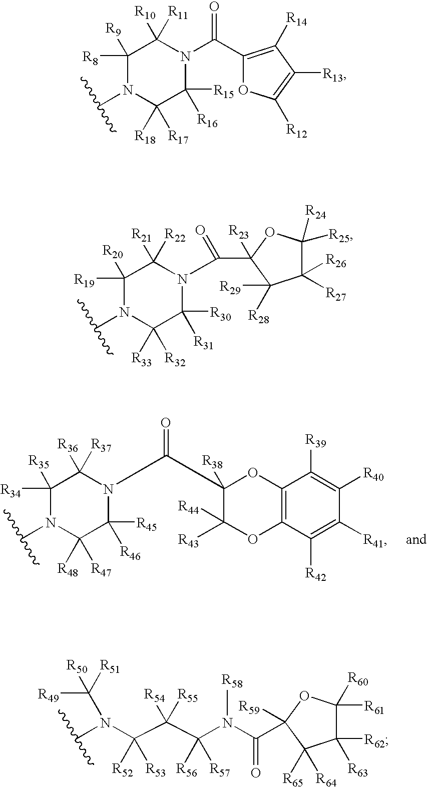
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%
with the proviso that compounds of Formula 1 cannot be selected from the group consisting of:

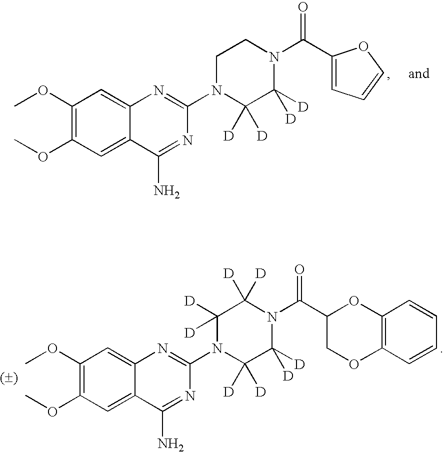

9. The method of claim 8, wherein said at least one polymorphically-expressed cytochrome P₄₅₀ isoform is selected from the group consisting of CYP2C8, CYP2C9, CYP2C19, and CYP2D6.

10. A method of treating a manual suffering from a disease or condition in which it is beneficial to modulate an alpha-adrenergic receptor, comprising administering a therapeutically effective amount of a compound of Formula 1 so as to affect a decreased inhibition of at least one cytochrome P₄₅₀ isoform in mammalian subjects per dosage unit thereof as compared to the non-isotopically enriched compound, wherein said compound of Formula 1 has the structure: 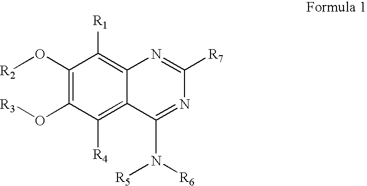
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 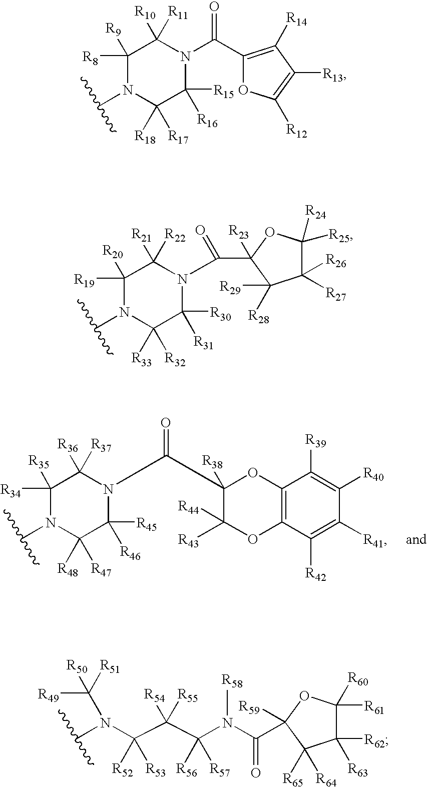
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%;
with the proviso that compounds of Formula 1 cannot be selected from the group consisting of:

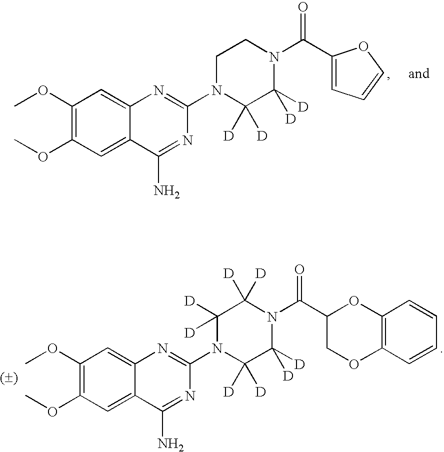

11. The method of claim 10, wherein said at least one cytochrome P₄₅₀ isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51,

12. A method of treating a mammal suffering from a disease or condition in which it is beneficial to modulate an alpha-adrenergic receptor, comprising administering a therapeutically effective amount of a compound of Formula 1 so as to elicit an improved clinical effect during the treatment in said mammal per dosage unit thereof as compared to the non-isotopically enriched compound;
wherein said compound of Formula 1 has the structure: 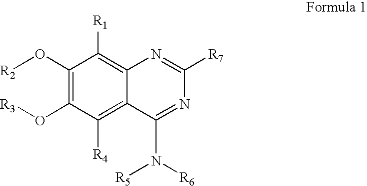
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 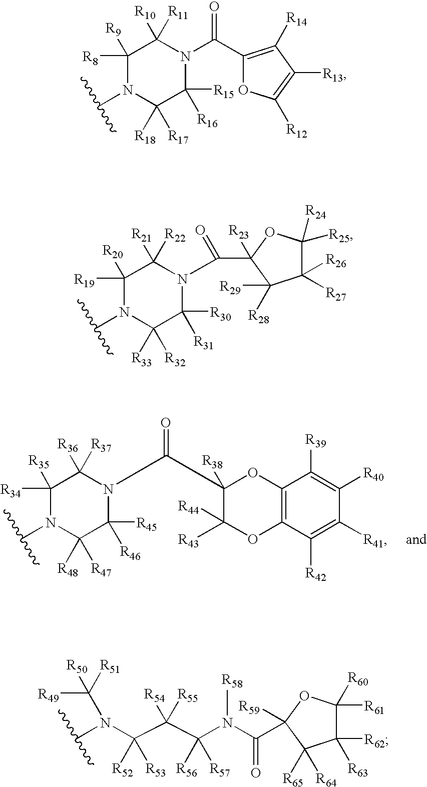
wherein R₈, R₉, R₁₀, R₁₁, R₁₂, R₁₃, R₁₄, K₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%;
with the proviso that compounds of Formula 1 cannot be selected from the group consisting of:

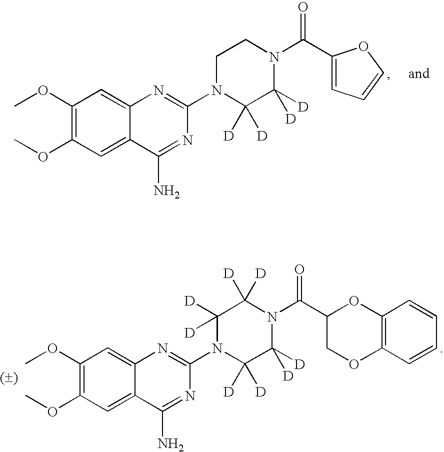

13. A method of treating a mammal suffering from a disease or condition in which it is beneficial to modulate an alpha-adrenergic receptor, comprising administering to said mammal a therapeutically effective amount of a compound of Formula 1;
wherein said compound of Formula 1 has the structure: 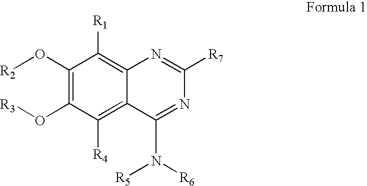
or a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof; or a pharmaceutically acceptable salt, solvate, or prodrug thereof wherein:
R₁, R₄, R₅, and R₆ are independently selected from the group consisting of hydrogen, and deuterium;
R₂ and R₃ are independently selected from the group consisting of —CH₃, —CH₂D, —CHD₂, and —CD₃;
R₇ is selected from the group consisting of: 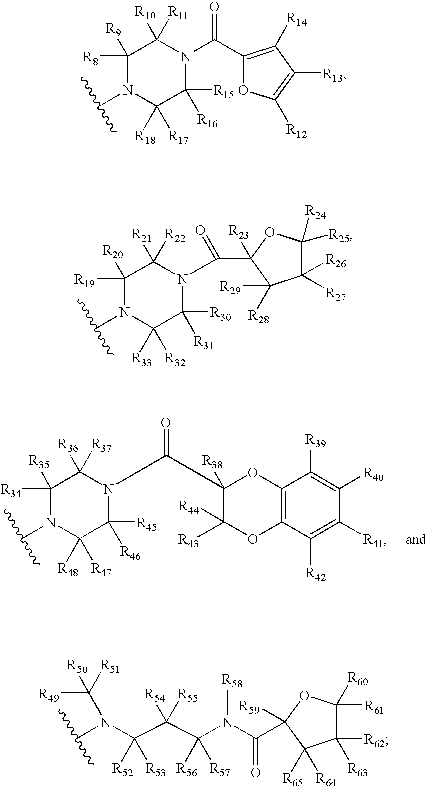
wherein R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, R₆₄, and R₆₅ are independently selected from the group consisting of hydrogen, and deuterium;
provided that said compound of Formula 1 contains at least one deuterium atom; and
provided that deuterium enrichment in said compound of Formula 1 is at least about 1%;
with the proviso that compounds of Formula 1 cannot be selected from the group consisting of:

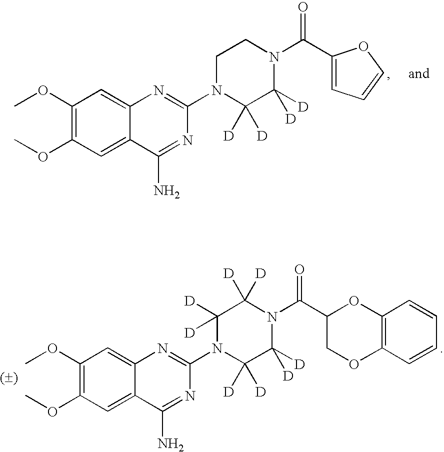

14. A pharmaceutical composition comprising a therapeutically effective amount of a compound according to claim 1, or a single enantiomer of a compound according to claim 1, a mixture of the (+)-enantiomer and the (−)-enantiomer of a compound according to claim 1, a mixture of about 90% or more by weight of the (−)-enantiomer and about 10% or less by weight of the (+)-enantiomer of a compound according to claim 1, a mixture of about 90% or more by weight of the (+)-enantiomer and about 10% or less by weight of the (−)-enantiomer of a compound according to claim 1, an individual diastereomer of a compound according to claim 1, or a mixture of diastereomers of a compound according to claim 1; or a pharmaceutically acceptable salt, solvate, or prodrug, with a pharmaceutically acceptable carrier or excipient.

15. The pharmaceutical composition of claim 14, wherein said composition is suitable for oral, parenteral, or intravenous infusion administration.

16. The pharmaceutical composition of claim 15, wherein said oral administration comprises administering a tablet or a capsule.

17. The pharmaceutical composition of claim 14, wherein said compound of claim 1 is administered in a dose of about 0.1 milligrams to about 100 milligrams total daily.

18. The compound of claim 4, wherein said compound contains a mixture of about 90% or more by weight of the (−)-enantiomer and about 10% or less by weight of the (+)-enantiomer.

19. The compound of claim 4, wherein said compound contains a mixture of about 90% or more by weight of the (+)-enantiomer and about 10% or less by weight of the (−)-enantiomer.

20. The method of claims 5, 6, 7, 8, 10, 12, or 13 wherein said disease or condition is selected from the group consisting of hypertension, cardiac failure, prostatitis, and benign prostatic hyperplasia.

21. The method of claims 5, 6, 7, 8, 10, 12, or 13 wherein the compound cannot be selected from the group consisting of:

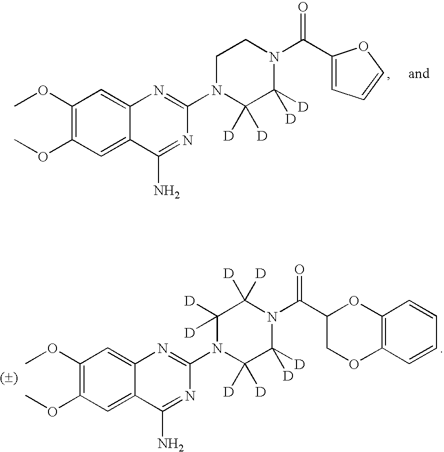

22. The compound of claim 1 selected from the group consisting of: 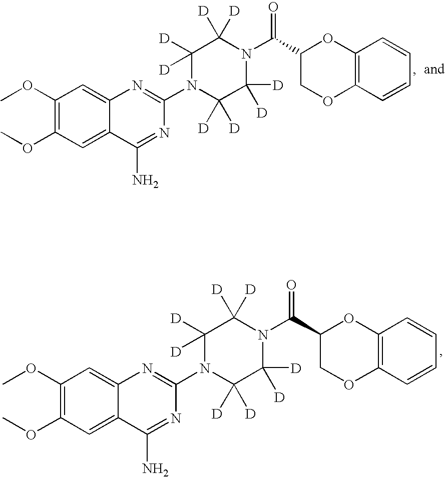
or a pharmaceutically acceptable salt, solvate or prodrug thereof.
